(19) United States
(12) Patent Application Publication
(10) Pub. No.: US 2002/0183309 A1
(43) Pub. Date: Dec. 5, 2002

(54) Novel amines as histamine-3 receptor ligands and their therapeutic applications

(76) Inventors: Marlon D. Cowart, Round Lake Beach, IL (US); Youssef L. Bennani, Shaker Heights, OH (US); Ramin Faghih, Lake Forest, IL (US); Lawrence A. Black, Libertyville, IL (US)

Correspondence Address: ABBOTT LABORATORIES, DEPT. 377 - AP6D-2, 100 ABBOTT PARK ROAD, ABBOTT PARK, IL, 60064-6050, US

(21) Appl. No.: 10/044,495

(22) Filed: Jan. 11, 2002

Related U.S. Application Data
(63) Continuation-in-part of application No. 09/810,648, filed on Mar. 16, 2001.
(60) Provisional application No. 60/276,793, filed on Mar. 16, 2001.

(51) Int. Cl. C 07D 4 9/02; C 07D 4 7/02; C07D 335/06; C07D 333/78; A61K 31/55; A61K 31/453; A61K 31/4535; A61K 31/4025

(52) U.S. Cl. 514/217.03; 514/320; 514/422; 514/432; 514/443; 514/457; 514/469; 540/596; 546/196; 548/525; 549/23; 549/49; 549/283

(57) ABSTRACT

Compounds of formula (I)

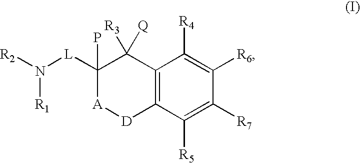

or a pharmaceutically acceptable salts or prodrug thereof which are useful for the modulation of the histamine-3 receptors in mammals and which are useful for the treatment of disorders ameliorated by histamine-3 receptor ligands.

This application is a continuation-in-part of U.S. patent application Ser. No. 09/810,648 and No. 60/276,793, filed Mar. 16, 2001.

### TECHNICAL FIELD

This invention relates to compounds which may be useful for treating diseases or conditions caused by or exacerbated by histamine-3 receptor activity, pharmaceutical compositions containing such compounds and methods of treatment using such compounds.

### BACKGROUND OF THE INVENTION

Histamine is a well-known mediator in hypersensitive reactions (e.g. allergies, hay fever, and asthma) which are commonly treated with antagonists of histamine or “antihistamines.” It has also been established that histamine receptors exist in at least two distinct types, referred to as H₁ and H₂ receptors.

A third histamine receptor (H₃ receptor) is believed to play a role in neurotransmission in the central nervous system, where the H₃ receptor is thought to be disposed presynaptically on histaminergic nerve endings (Nature, 302, 832-837 (1983)). The existence of the H₃ receptor has been confirmed by the development of selective H₃ receptor agonists and antagonists (Nature, 327, 117-123 (1987)) and has subsequently been shown to regulate the release of other neurotransmitters in both the central nervous system and peripheral organs, particularly the lungs, cardiovascular system and gastrointestinal tract.

A number of diseases or conditions may be treated with histamine-3 receptor ligands wherein the H₃ ligand may be an antagonist, agonist or partially agonist. Such diseases or conditions include cardiovascular disorders such as acute myocardial infarction; memory processes, dementia and cognition disorders such as Alzheimer's disease and attention-deficit hyperactivity disorder; neurological disorders such as Parkinson's disease, schizophrenia, depression, epilepsy, and seizures or convulsions; cancer such as cutaneous carcinoma, medullary thyroid carcinoma and melanoma; respiratory disorders such as asthma; sleep disorders such as narcolepsy; vestibular dysfunction such as Meniere's disease; gastrointestinal disorders, inflammation, migraine, motion sickness, obesity, pain, and septic shock.

### SUMMARY OF INVENTION

In its principle embodiment, the present invention is directed to compounds of formula (I):

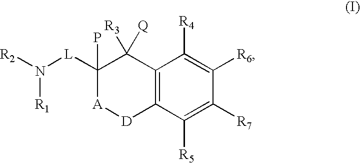

or a pharmaceutically acceptable salt or prodrugs thereof, wherein

A is selected from carbonyl or covalent bond;

D is selected from O or S;

L is selected from —CH₂CH₂— or —CH₂CH₂CH₂—;

P and Q taken together form a covalent bond or are both hydrogen;

R₁ and R₂ are each independently selected from hydrogen, alkyl, aryl, arylalkyl, cycloalkyl, cycloalkylalkyl, heterocycle, heterocyclealkyl, hydroxyalkyl, alkenyl, or alkynyl; or

R₁ and R₂ taken together with the nitrogen atom to which they are attached, together form a heterocycle;

R₃, R₄ and R₅ are each independently selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

R₆ and R₇ are each independently selected from the group consisting of hydrogen, alkoxy, alkoxycarbonyl, alkyl, alkylcarbonyl, alkylcarbonyloxy, alkylsulfinyl, alkylsulfonyl, alkylthio, aryl, carboxy, carboxyalkyl, cyano, cyanoalkyl, cycloalkyl, formyl, halogen, haloalkoxy, haloalkyl, heterocycle, hydroxy, hydroxyalkyl, mercapto, nitro, —NR_{A}R_{B}, (NR_{A}R_{B})alkyl, (NR_{A}R_{B})carbonyl, (NR_{A}R_{B})sulfonyl or —L₂R₂₀;

L₂ is selected from alkylene, alkenylene, O, S, C(═O), N(═OR₂₁) or N(R_{A});

R₂₀ is selected from aryl, heterocycle or cycloalkyl;

R₂₁ is selected from hydrogen or alkyl; and

R_{A} and R_{B} are each independently selected from hydrogen, alkyl, alkylcarbonyl or formyl;

provided that at least one, but not both, of R₆ or R₇ is aryl, heterocycle, cycloalkyl or L₂R₂₀.

In another embodiment, compounds of the present invention have formula (I) wherein A is a covalent bond; D is O; L is —CH₂CH₂—; P and Q taken together form a covalent bond; R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, (2R)-2-methyl-1-pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; R₃, R₄, R₅ and R₇ are hydrogen; and R₆ is L₂R₂₀; L₂ is C(═O); and R₂₀ is aryl.

In another embodiment, compounds of the present invention have formula (I) wherein A is a covalent bond; D is O; L is —CH₂CH₂—; P and Q taken together form a covalent bond; R₁ and R₂ taken together with the nitrogen atom to which they are attached, together form a heterocycle wherein said heterocycle is (2R)-2-methyl-1-pyrrolidinyl; R₃, R₄, R₅ and R₇ are hydrogen; R₆ is L₂R₂₀; L₂ is C(═O); and R₂₀ is aryl wherein said aryl is phenyl wherein said phenyl can be substituted with 1, 2 or 3 substitutents selected from the group consisting of alkoxy, alkyl, alkylthio, halogen and —NR_{A}R_{B}.

In another embodiment, compounds of the present invention have formula (I) wherein A is a covalent bond; D is O; L is —CH₂CH₂—; P and Q taken together form a covalent bond; R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, (2R)-2-methyl-1-pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; R₃, R₄, R₅ and R₇ are hydrogen; R₆ is L₂R₂₀; L₂ is C(═O); and R₂₀ is cycloalkyl.

In another embodiment, compounds of the present invention have formula (I) wherein A is a covalent bond; D is O; L is —CH₂CH₂—; P and Q taken together form a covalent bond; R₁ and R₂ taken together with the nitrogen atom to which they are attached, together form a heterocycle wherein said heterocycle is (2R)-2-methyl-1-pyrrolidinyl; R₃, R₄, R₅ and R₇ are hydrogen; R₆ is L₂R₂₀; L₂ is C(═O); and R₂₀ is cycloalkyl.

In another embodiment, compounds of the present invention have formula (I) wherein A is a covalent bond; D is O; L is —CH₂CH₂—; P and Q taken together form a covalent bond; R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, (2R)-2-methyl-1-pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; R₃, R₄, R₅ and R₇ are hydrogen; and R₆ is alkylcarbonyl.

In another embodiment, compounds of the present invention have formula (I) wherein A is a covalent bond; D is O; L is —CH₂CH₂—; P and Q taken together form a covalent bond; R₁ and R₂ taken together with the nitrogen atom to which they are attached, together form a heterocycle wherein said heterocycle is (2R)-2-methyl-1-pyrrolidinyl; R₃, R₄, R₅ and R₇ are hydrogen; and R₆ is alkylcarbonyl.

According to another embodiment, compounds of the present invention have formula (II)

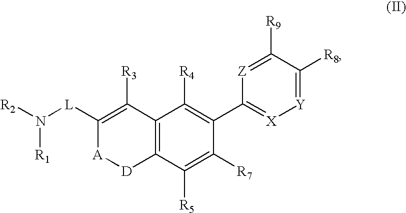

or a pharmaceutical acceptable salt or prodrug thereof, wherein

R₇ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

R₈ is selected from hydrogen, alkylcarbonyl, arylcarbonyl, cyano, cycloalkylcarbonyl, heterocyclecarbonyl or (NR_{A}R_{B})carbonyl;

R₉ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

X is selected from CH, CR_{X} or N;

Y is selected from CH, CR_{Y} or N;

Z is selected from CH, CR_{Z} or N;

R_{X}, R_{Y} and R_{Z} groups are each independently selected from alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl; and

A, D, L, R_{A}, R_{B}, R₁, R₂, R₃, R4 and R₅ are as defined in formula (I).

In another embodiment, compounds of the present invention have formula (II) wherein A is a covalent bond; and D, L, R_{A}, R_{B}, R₁, R₂, R₃, R₄, R₅, R₇, R₈, R₉, X, Y, and Z are as defined in formula (I).

In another embodiment, compounds of the present invention have formula (II) wherein A is a covalent bond, R₁ and R₂ are each independently selected from hydrogen, alkyl, hydroxyalkyl, alkenyl or alkynyl; R₈ is cyano; and D, L, R_{A}, R_{B}, R₃, R₄, R₅, R₇ and R₉, X, Y, and Z are as defined in formula (I).

In another embodiment, compounds of the present invention have formula (II) wherein L is —CH₂CH₂—; A is a covalent bond; R₁ and R₂ are each independently selected from hydrogen, alkyl, hydroxyalkyl, alkenyl or alkynyl; R₃, R₄, R₅, R₇ and R₉ are hydrogen; R₈ is cyano; X is CH; Y is CH; Z is CH; and D, R_{A} and R_{B} are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is cyano; X is nitrogen; Y is CH; Z is CH; and D, L, R_{A}, R_{B}, R₃, R₄, R₅, R₇ and R₉ are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is heterocyclecarbonyl; and D, L, R_{A}, R_{B}, R₃, R₄, R₅, R₇, R₉, X, Y and Z are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₇, R₉, X, Y and Z are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is heterocyclecarbonyl wherein the heterocycle part of said heterocyclecarbonyl is morpholine; and D, L, R_{A}, R_{B}, R₃, R₄, R₅, R₇, R₉, X, Y and Z are as defined in formula (I).

In another embodiment, compounds of the present invention have formula (II) wherein A is a covalent bond; L is —CH₂CH₂—; R₁ and R₂ are each independently selected from hydrogen, alkyl, hydroxyalkyl, alkenyl or alkynyl; R₃, R₄, R₅, R₇ and R₉ are hydrogen; R₈ is heterocyclecarbonyl wherein the heterocycle part of said heterocyclecarbonyl is morpholine; X is CH; Y is CH; Z is CH; and D, R_{A} and R_{B} are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is heterocyclecarbonyl; X is nitrogen; Y is CH; Z is CH; and D, L, R_{A}, R_{B}, R₃, R₄, R₅, R₇ and R₉ are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; X is nitrogen; Y is CH; Z is CH; and D, L, R_{A}, R_{B}, R₃, R₄, R₅, R₇ and R₉ are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is heterocyclecarbonyl wherein the heterocycle part of said heterocyclecarbonyl is morpholinyl; X is nitrogen; Y is CH; Z is CH; and D, L, R_{A}, R_{B}, R₃, R4, R₅, R₇ and R₉ are as defined in formula (I).

In another embodiment, compounds of the present invention have formula (II) wherein A is a covalent bond; L is —CH₂CH₂—; R₁ and R₂ are each independently selected from hydrogen, alkyl, hydroxyalkyl, alkenyl or alkynyl; R₃, R₄, R₅, R₇ and R₉ are hydrogen; R₈ is heterocyclecarbonyl wherein the heterocycle part of said heterocyclecarbonyl is morpholinyl; X is nitrogen; Y is CH; Z is CH; and D, R_{A} and R_{B} are as defined in formula (I).

In another embodiment, compounds of the present invention have formula (II) wherein A is a covalent bond; R₁ and R₂ taken together with the nitrogen atom to which they are attached, together form a heterocycle and D, L, R_{A}, R_{B}, R₃, R₄, R₅, R₇, R₈, R₉, X, Y and Z are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is cyano; and D, L, R_{A}, R_{B}, R₃, R₄, R₅, R₇, R₉, X, Y and Z are as defined in formula (I).

In another embodiment, compounds of the present invention have formula (II) wherein A is a covalent bond; L is —CH₂CH₂—; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₃, R₄, R₅, R₇, R₉ are hydrogen; R₈ is cyano; X is CH; Y is CH; Z is CH; and D, R_{A} and R_{B} are as defined in formula (I).

In another embodiment, compounds of the present invention have formula (II) wherein A is a covalent bond; R₁ and R₂ taken together with the nitrogen atom to which they are attached, together form a heterocycle; R₈ is cyano; X is nitrogen; Y is CH; Z is CH; and D, L, R_{A}, R_{B}, R₃, R₄, R₅, R₇ and R₉ are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is cyano; X is nitrogen; Y is CH; Z is CH; and D, L, R_{A}, R_{B}, R₃, R₄, R₅, R₇ and R₉ are as defined in formula (I).

In another embodiment, compounds of the present invention have formula (II) wherein A is a covalent bond; R₁ and R₂ taken together with the nitrogen atom to which they are attached, together form a heterocycle; R₈ is heterocyclecarbonyl; and D, L, R_{A}, R_{B}, R₃, R₄, R₅, R₇, R₉, X, Y, and Z are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₇, R₉, X, Y, and Z are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is heterocyclecarbonyl wherein the heterocycle part of said heterocyclecarbonyl is morpholinyl; and D, L, R_{A}, R_{B}, R₃, R₄, R₅, R₇, R₉, X, Y, and Z are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1 ,1-dioxidothiomorpholinyl; R₃, R₄, R₅, R₇ and R₉ are hydrogen; R₈ is heterocyclecarbonyl wherein the heterocycle part of said heterocyclecarbonyl is morpholinyl; X is CH; Y is CH; Z is CH; and D, L, R_{A} and R_{B} are as defined in formula (I).

In another embodiment, compounds of the present invention have formula (II) wherein A is a covalent bond; R₁ and R₂ taken together with the nitrogen atom to which they are attached, together form a heterocycle; R₈ is heterocyclecarbonyl; X is nitrogen; Y is CH; Z is CH; and D, L, R_{A}, R_{B}, R₃, R₄, R₅, R₇, and R₉ are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; X is nitrogen; Y is CH; Z is CH; and D, L, R_{A}, R_{B}, R₃, R₄, R₅, R₇, and R₉ are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or l,l-dioxidothiomorpholinyl; R₈ is heterocyclecarbonyl wherein the heterocycle part of said heterocyclecarbonyl is morpholinyl; X is nitrogen; Y is CH; Z is CH; and D, L, R_{A}, R_{B}, R₃, R₄, R₅,R₇, and R₉ are as defined in formula (I).

In another embodiment, compounds of the present invention have formula (II) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₃, R₄, R₅, R₇ and R₉ are hydrogen; R₈ is heterocyclecarbonyl wherein the heterocycle part of said heterocyclecarbonyl is morpholinyl; X is nitrogen; Y is CH; Z is CH; and D, L, R_{A}, R_{B}, R₃, R₄, R₅, R₇, and R₉ are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is selected from cyano or heterocyclecarbonyl; and D, L, R_{A}, R_{B}, R₃, R₄, R₅, R₇, R₉, X, Y and Z are as defined in formula (I).

In another embodiment, compounds of the present invention have formula (II) wherein A is a covalent bond; R₁ and R₂ are each independently selected from hydrogen, alkyl, hydroxyalkyl, alkenyl or alkynyl; R₈ is selected from cyano or heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₇, R₉, X, Y and Z are as defined in formula (I).

In another embodiment, compounds of the present invention have formula (II) wherein A is carbonyl; R₁ and R₂ taken together with the nitrogen atom to which they are attached, together form a heterocycle; R₈ is selected from cyano or heterocyclecarbonyl; and D, L, R_{A}, R_{B}, R₃, R₄, R₅, R₇, R₉, X, Y and Z are as defined in formula (I).

In another embodiment, compounds of the present invention have formula (II) wherein A is carbonyl; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is selected from the group consisting of cyano, or heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₇, R₉, X, Y and Z are as defined in formula (I).

According to another embodiment, compounds of the present invention have formula (III)

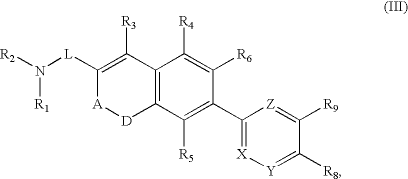

or a pharmaceutical acceptable salt or prodrug thereof, wherein

R₆ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

R₈ is selected from hydrogen, alkylcarbonyl, arylcarbonyl, cyano, cycloalkylcarbonyl, heterocyclecarbonyl or (NR_{A}R_{B})carbonyl;

R₉ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

X is selected from CH, CR_{X} or N;

Y is selected from CH, CR_{Y} or N;

Z is selected from CH, CR_{Z} or N;

R_{X}, R_{Y} and R_{Z} are each independently selected from alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl; and

A, D, L, R_{A}, R_{B}, R₁, R₂, R₃, R₄, and R₅ are as defined in formula (I).

In another embodiment, compounds of the present invention have formula (III) wherein A is a covalent bond; and D, L, R_{A}, R_{B}, R₁, R₂, R₃, R₄, R₅, R₆, R₈R₉, X, Y, and Z are as defined in formula (I).

In another embodiment, compounds of the present invention have formula (III) wherein A is a covalent bond; R₁ and R₂ are each independently selected from hydrogen, alkyl, hydroxyalkyl, alkenyl or alkynyl; R₈ is selected from cyano or heterocyclecarbonyl; and D, L, R_{A}, R_{B}, R₃, R₄, R₅, R₆, R₉, X, Y, and Z are as defined in formula (I).

In another embodiment, compounds of the present invention have formula (III) wherein A is a covalent bond; R₁ and R₂ are each independently selected from hydrogen, alkyl, hydroxyalkyl, alkenyl and alkynyl; R₈ is selected from cyano or heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₆, R₉, X, Y, and Z are as defined in formula (I).

In another embodiment, compounds of the present invention have formula (III) wherein A is a covalent bond; R₁ and R₂ taken together with the nitrogen atom to which they are attached, together form a heterocycle; R₈ is selected from cyano or heterocyclecarbonyl; and D, L, R_{A}, R_{B}, R₃, R₄, R₅, R₆, R₉, X, Y, and Z are as defined in formula (I).

In another embodiment, compounds of the present invention have formula (III) wherein A is a covalent bond; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is selected from cyano or heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₆, R₉, X, Y, and Z are as defined in formula (I).

In another embodiment, compounds of the present invention have formula (III) wherein A is carbonyl; and D, L, R_{A}, R_{B}, R₁, R₂, R₃, R₄, R₅, R₆, R₈, R₉, X, Y, and Z are as defined in formula (I).

In another embodiment, compounds of the present invention have formula (III) wherein A is carbonyl; R₁ and R₂ are each independently selected from hydrogen, alkyl, hydroxyalkyl, alkenyl or alkynyl; R₈ is cyano; and D, L, R_{A}, R_{B}, R₃, R₄, R₅, R₆, R₉, X, Y, and Z are as defined in formula (I).

In another embodiment, compounds of the present invention have formula (III) wherein L is —CH₂CH₂—; A is carbonyl; R₁ and R₂ are each independently selected from hydrogen, alkyl, hydroxyalkyl, alkenyl or alkynyl; R₃ is methyl, R₄, R₅, R₆ and R₉ are hydrogen; R₈ is cyano; X is CH; Y is CH; Z is CH; and D, R_{A} and R_{B} are as defined in formula (I).

In another embodiment, compounds of the present invention have formula (III) wherein A is carbonyl; R₁ and R₂ are each independently selected from hydrogen, alkyl, hydroxyalkyl, alkenyl or alkynyl; R₈ is heterocyclecarbonyl; and D, L, R_{A}, R_{B}, R₃, R₄, R₅, R₆, R₉, X, Y, and Z are as defined in formula (I).

In another embodiment, compounds of the present invention have formula (III) wherein A is carbonyl; R₁ and R₂ are each independently selected from hydrogen, alkyl, hydroxyalkyl, alkenyl or alkynyl; R₈ is heterocyclecarbonyl wherein the heterocycle part of said heterocyclecarbonyl is selected from azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₆, R₉, X, Y, and Z are as defined in formula (I).

In another embodiment, compounds of the present invention have formula (Ill) wherein A is carbonyl; R₁ and R₂ taken together with the nitrogen atom to which they are attached, together form a heterocycle; R₈ is cyano; and D, L, R_{A}, R_{B}, R₃, R₄, R₅, R₆, R₉, X, Y, and Z are as defined in formula (I).

In another embodiment, compounds of the present invention have formula (III) wherein A is carbonyl; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is cyano; and D, L, R_{A}, R_{B}, R₃, R₄, R₅, R₆, R₉, X, Y, and Z are as defined in formula (I).

In another embodiment, compounds of the present invention have formula (III) wherein L is —CH₂CH₂—; A is carbonyl; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₃ is methyl; R₄, R₅, R₆ and R₉ are hydrogen; R₈ is cyano; X is CH; Y is CH; Z is CH; and D, L, R_{A} and R_{B} are as defined in formula (I).

In another embodiment, compounds of the present invention have formula (III) wherein A is carbonyl; R₁ and R₂ taken together with the nitrogen atom to which they are attached, together form a heterocycle; R₈ is heterocyclecarbonyl; and D, L, R_{A}, R_{B}, R₃, R₄, R₅, R₆, R₈, X, Y, and Z are as defined in formula (I).

In another embodiment, compounds of the present invention have formula (III) wherein A is carbonyl; R₁ and R₂ taken together with the nitrogen atom to which they are attached, together form a heterocycle selected from azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; R₈ is heterocyclecarbonyl wherein the heterocycle part of said heterocyclecarbonyl is selected from azetidine, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl or 1,1-dioxidothiomorpholinyl; and D, L, R_{A}, R_{B}, R₃, R₄, R₅, R₆, R₉, X, Y, and Z are as defined in formula (I).

According to another embodiment, compounds of the present invention have formula (IV)

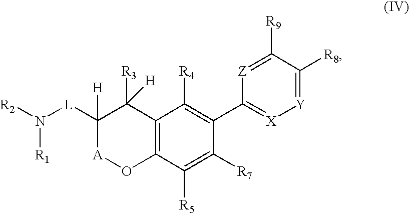

or a pharmaceutical acceptable salt or prodrug thereof, wherein

R₇ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

R₈ is selected from hydrogen, alkylcarbonyl, arylcarbonyl, cyano, cycloalkylcarbonyl, heterocyclecarbonyl or (NR_{A}R_{B})carbonyl;

R₉ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

X is selected from CH, CR_{X} or N;

Y is selected from CH, CR_{Y} or N;

Z is selected from CH, CR_{Z} or N;

R_{X}, R_{Y} and R_{Z} are each independently selected from alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}, R_{B}, (NR_{A}R_{B})alkyl, (NR_{A}R_{B})carbonyl or (NR_{A}R_{B})sulfonyl; and

D, L, R_{A}, R_{B}, R₁, R₂, R₃, R₄ and R₅ are as defined in formula (I).

In another embodiment, compounds of the present invention have formula (IV) wherein A is a covalent bond; and D, L, R_{A}, R_{B}, R₁, R₂, R₃, R₄, R₅, R₇, R₈, R₉, X, Y, and Z are as defined in formula (I).

In another embodiment, compounds of the present invention have formula (IV) wherein A is a covalent bond; R₁ and R₂ are each independently selected from hydrogen, alkyl, hydroxyalkyl, alkenyl or alkynyl; R₈ is cyano; and D, L, R_{A}, R_{B}, R₃, R₄, R₅, R₇, R₉, X, Y, and Z are as defined in formula (I).

In another embodiment, compounds of the present invention have formula (IV) wherein L is —CH₂CH₂—; A is a covalent bond; R₁ and R₂ are each independently selected from hydrogen, alkyl, hydroxyalkyl, alkenyl or alkynyl; R₃, R₄, R₅, R₇ and R₈ are hydrogen; R₈ is cyano; X is CH; Y is CH; Z is CH; and D, R_{A} and R_{B} are as defined in formula (I).

In a further embodiment, compounds of the present invention have formula (V)

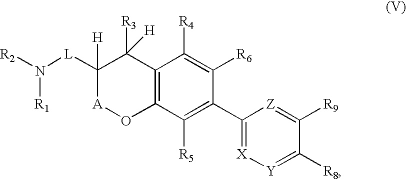

or a pharmaceutical acceptable salt or prodrug thereof, wherein

R₆ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

R₈ is selected from hydrogen, alkylcarbonyl, arylcarbonyl, cyano, cycloalkylcarbonyl, heterocyclecarbonyl or (NR_{A}R_{B})carbonyl;

R₈ is selected from hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl;

X is selected from CH, CR_{X} or N;

Y is selected from CH, CR_{Y} or N;

Z is selected from CH, CR_{Z} or N;

R_{X}, R_{Y} and R_{Z} are each independently selected from alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl or (NR_{A}R_{B})sulfonyl; and

A, D, L, R_{A}, R_{B}, R₁, R₂, R₃, R₄, R₅ are as defined in formula (I).

### DETAILED DESCRIPTION OF THE INVENTION

The present invention discloses compounds of formula (I-V) which are useful for selectively modulating the effects of the histamine-3 receptors in a mammal. According to one embodiment of the present invention, there is provided a method of treating a disorder wherein the disorder is ameliorated by modulating the histamine-3 receptors in a mammal. According to another embodiment of the present invention, there is provided a method of treating a disorder wherein the disorder is selected from the group consisting of acute myocardial infarction, asthma, bipolar disorder, cognitive enhancement, cognitive deficits in psychiatric disorders, cutaneous carcinoma, drug abuse, depression, gastrointestinal disorders, inflammation, jet lag, medullary thyroid carcinoma, melanoma, Meniere's disease, migraine, mood and attention alteration, motion sickness, neurogenic inflammation, obsessive compulsive disorder, pain, Parkinson's disease, schizophrenia, seizures, septic shock, Tourette's syndrome, vertigo, or wakefulness. According to another embodiment of the present invention, there is provided a method of treating a disorder wherein the disorder is Alzheimer's disease. According to another embodiment of the present invention, there is provided a method of treating a disorder wherein the disorder is attention-deficit hyperactivity disorder. According to another embodiment of the present invention, there is provided a method of treating a disorder wherein the disorder is epilepsy. According to another embodiment of the present invention, there is provided a method of treating a disorder wherein the disorder is narcolepsy. According to another embodiment of the present invention, there is provided a method of treating a disorder wherein the disorder is selected from mild cognitive impairment, deficits of memory, deficits of learning and dementia.

### Definition of Terms

As used for the present invention, the following terms have the meanings ascribed.

The term “alkenyl,” as used herein, refers to a straight or branched chain hydrocarbon containing from 2 to 10 carbons and containing at least one carbon-carbon double bond formed by the removal of two hydrogens. Representative examples of alkenyl include, but are not limited to, ethenyl, 2-propenyl, 2-methyl-2-propenyl, 3-butenyl, 4-pentenyl, 5-hexenyl, 2-heptenyl, 2-methyl-1-heptenyl, and 3-decenyl.

The term “alkenylene” means a divalent group derived from a straight or branched chain hydrocarbon of from 2 to 10 carbon atoms containing at least one double bond. Representative examples of alkenylene include, but are not limited to, —CH═CH—, —C(═CH₂)— —CH═CH₂CH₂—, —CH₂CH₂C(═CH₂)CH₂—, —CH₂CH₂C(═CHCH₃)CH₂—, and —CH═C(CH₃)CH₂—.

The term “alkoxy,” as used herein, refers to an alkyl group, as defined herein, appended to the parent molecular moiety through an oxy moiety, as defined herein. Representative examples of alkoxy include, but are not limited to, methoxy, ethoxy, propoxy, 2-propoxy, butoxy, tert-butoxy, pentyloxy, and hexyloxy.

The term “alkoxyalkyl,” as used herein, refers to an alkoxy group, as defined herein, appended to the parent molecular moiety through an alkyl group, as defined herein. Representative examples of alkoxyalkyl include, but are not limited to, tert-butoxymethyl, 2-ethoxyethyl, 2-methoxyethyl and methoxymethyl.

The term “alkoxycarbonyl,” as used herein, refers to an alkoxy group, as defined herein, appended to the parent molecular moiety through a carbonyl group, as defined herein. Representative examples of alkoxycarbonyl include, but are not limited to, methoxycarbonyl, ethoxycarbonyl and tert-butoxycarbonyl.

The term “alkyl,” as used herein, refers to a straight or branched chain hydrocarbon containing from 1 to 10 carbon atoms. Representative examples of alkyl include, but are not limited to, methyl, ethyl, n-propyl, iso-propyl, n-butyl, sec-butyl, iso-butyl, tert-butyl, n-pentyl, isopentyl, neopentyl, n-hexyl, 3-methylhexyl, 2,2-dimethylpentyl, 2,3-dimethylpentyl, n-heptyl, n-octyl, n-nonyl and n-decyl.

The term “alkylcarbonyl,” as used herein, refers to an alkyl group, as defined herein, appended to the parent molecular moiety through a carbonyl group, as defined herein. Representative examples of alkylcarbonyl include, but are not limited to, acetyl, 1-oxopropyl, 2,2-dimethyl-1-oxopropyl, 1-oxobutyl and 1-oxopentyl.

The term “alkylcarbonyloxy,” as used herein, refers to an alkylcarbonyl group, as defined herein, appended to the parent molecular moiety through an oxy moiety, as defined herein. Representative examples of alkylcarbonyloxy include, but are not limited to, acetyloxy, ethylcarbonyloxy and tert-butylcarbonyloxy.

The term “alkylene” means a divalent group derived from a straight or branched chain hydrocarbon of from 1 to 10 carbon atoms. Representative examples of alkylene include, but are not limited to, —CH₂—, —CH₂CH₂—, —CH₂CH₂CH₂—, —CH₂CH₂CH₂CH₂—, and —CH₂CH(CH₃)CH₂—.

The term “alkylsulfinyl,” as used herein, refers to an alkyl group, as defined herein, appended to the parent molecular moiety through a sulfinyl group, as defined herein. Representative examples of alkylsulfinyl include, but are not limited to, methylsulfinyl and ethylsulfinyl.

The term “alkylsulfonyl,” as used herein, refers to an alkyl group, as defined herein, appended to the parent molecular moiety through a sulfonyl group, as defined herein. Representative examples of alkylsulfonyl include, but are not limited to, ethylsulfonyl, isopropylsulfonyl and methylsulfonyl.

The term “alkylthio,” as used herein, refers to an alkyl group, as defined herein, appended to the parent molecular moiety through a sufur atom, as defined herein. Representative examples of alkylthio include, but are not limited to, methylsulfanyl, ethylsulfanyl, tert-butylsulfanyl and hexylsulfanyl.

The term “aryl,” as used herein, refers to a monocyclic-ring system, or a bicyclic- or a tricyclic- fused ring system wherein one or more of the fused rings are aromatic. Representative examples of aryl include, but are not limited to, anthracenyl, azulenyl, fluorenyl, indanyl, indenyl, naphthyl, phenyl, and tetrahydronaphthyl.

The aryl groups of this invention can be substituted with 1, 2, 3, 4 or 5 substituents independently selected from alkenyl, alkoxy, alkoxyalkoxy, alkoxyalkyl, alkoxycarbonyl, alkyl, alkylcarbonyl, alkylcarbonyloxy, alkylsulfinyl, alkylsulfonyl, alkylthio, alkynyl, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl, and (NR_{A}R_{B})sulfonyl.

The term “arylalkyl,” as used herein, refers to an aryl group, as defined herein, appended to the parent molecular moiety through an alkyl group, as defined herein. Representative examples of arylalkyl include, but are not limited to, benzyl, 2-phenylethyl, 3-phenylpropyl, and 2-naphth-2-ylethyl.

The term “arylcarbonyl,” as used herein, refers to an aryl group, as defined herein, appended to the parent molecular moiety through a carbonyl group, as defined herein. Representative examples of arylcarbonyl include, but are not limited to, benzoyl, phenylacetyl, 3-chlorophenylacetyl, 3-methoxyphenylacetyl, 4-fluoro-3-methylphenylacetyl, 3-phenylpropionyl, and 2-naphthylacetyl.

The term “carbonyl,” as used herein, refers to a —C(O)— group.

The term “carboxy,” as used herein, refers to a —CO₂H group.

The term “carboxyalkyl,” as used herein, refers to a carboxy group, as defined herein, appended to the parent molecular moiety through an alkyl group, as defined herein. Representative examples of carboxyalkyl include, but are not limited to, carboxymethyl, 2-carboxyethyl, and 3-carboxypropyl.

The term “cyano,” as used herein, refers to a —CN group.

The term “cyanoalkyl,” as used herein, refers to a cyano group, as defined herein, appended to the parent molecular moiety through an alkyl group, as defined herein. Representative examples of cyanoalkyl include, but are not limited to, cyanomethyl, 2-cyanoethyl and 3-cyanopropyl.

The term “cycloalkyl,” as used herein, refers to a saturated cyclic hydrocarbon group containing from 3 to 8 carbons. Examples of cycloalkyl include cyclopropyl, cyclobutyl, cyclopentyl, cyclohexyl, cycloheptyl and cyclooctyl .

The term “cycloalkylalkyl,” as used herein, refers to cycloalkyl group, as defined herein, appended to the parent molecular moiety through an alkyl group, as defined herein. Representative examples of cycloalkylalkyl include, but are not limited to, cyclopropylmethyl, 2-cyclobutylethyl, cyclopentylmethyl, cyclohexylmethyl and 4-cycloheptylbutyl.

The term “formyl,” as used herein, refers to a —C(O)H group.

The term “halo” or “halogen,” as used herein, refers to —Cl, —Br, —I or —F.

The term “haloalkoxy,” as used herein, refers to at least one halogen, as defined herein, appended to the parent molecular moiety through an alkoxy group, as defined herein. Representative examples of haloalkoxy include, but are not limited to, chloromethoxy, 2-fluoroethoxy, trifluoromethoxy, and pentafluoroethoxy.

The term “haloalkyl,” as used herein, refers to at least one halogen, as defined herein, appended to the parent molecular moiety through an alkyl group, as defined herein. Representative examples of haloalkyl include, but are not limited to, chloromethyl, 2-fluoroethyl, trifluoromethyl, pentafluoroethyl, and 2-chloro-3-fluoropentyl.

The term “heterocycle” or “heterocyclic,” as used herein, refers to a monocyclic or bicyclic ring system. Monocyclic ring systems are exemplified by any 3- or 4-membered ring containing a heteroatom independently selected from oxygen, nitrogen and sulfur; or a 5-, 6- or 7-membered ring containing one, two or three heteroatoms wherein the heteroatoms are independently selected from nitrogen, oxygen and sulfur. The 5-membered ring has from 0-2 double bonds and the 6- and 7-membered rings have from 0-3 double bonds. Representative examples of monocyclic ring systems include, but are not limited to, azetidinyl, azepanyl, aziridinyl, diazepinyl, 1,3-dioxolanyl, dioxanyl, dithianyl, furyl, imidazolyl, imidazolinyl, imidazolidinyl, isothiazolyl, isothiazolinyl, isothiazolidinyl, isoxazolyl, isoxazolinyl, isoxazolidinyl, morpholinyl, oxadiazolyl, oxadiazolinyl, oxadiazolidinyl, oxazolyl, oxazolinyl, oxazolidinyl, piperazinyl, piperidinyl, pyranyl, pyrazinyl, pyrazolyl, pyrazolinyl, pyrazolidinyl, pyridyl, pyrimidinyl, pyridazinyl, 2,5-dihydro-1H-pyrrolyl, pyrrolyl, pyrrolinyl, pyrrolidinyl, tetrahydrofuranyl, tetrahydrothienyl, tetrazinyl, tetrazolyl, thiadiazolyl, thiadiazolinyl, thiadiazolidinyl, thiazolyl, thiazolinyl, thiazolidinyl, thienyl, thiomorpholinyl, 1,1-dioxidothiomorpholinyl (thiomorpholine sulfone), thiopyranyl, triazinyl, triazolyl, and trithianyl. Bicyclic ring systems are exemplified by any of the above monocyclic heterocyclic ring systems fused to an aryl group as defined herein, a cycloalkyl group as defined herein, or another monocyclic heterocyclic ring system. Representative examples of bicyclic ring systems include but are not limited to, benzimidazolyl, benzothiazolyl, benzothienyl, benzoxazolyl, benzofuranyl, benzopyranyl, benzothiopyranyl, benzodioxinyl, 1,3-benzodioxolyl, cinnolinyl, indazolyl, indolyl, indolinyl, indolizinyl, naphthyridinyl, isobenzofuranyl, isobenzothienyl, isoindolyl, isoindolinyl, isoquinolinyl, phthalazinyl, pyranopyridyl, quinolinyl, quinolizinyl, quinoxalinyl, quinazolinyl, tetrahydroisoquinolinyl, tetrahydroquinolinyl, and thiopyranopyridyl.

The heterocycles of this invention can be substituted with 1, 2, or 3 substituents independently selected from alkenyl, alkoxy, alkoxyalkyl, alkoxycarbonyl, alkyl, alkylcarbonyl, alkylcarbonyloxy, alkylsulfinyl, alkylsulfonyl, alkylthio, arylalkyl, carboxy, carboxyalkyl, cyano, cyanoalkyl, formyl, halogen, haloalkoxy, haloalkyl, hydroxy, hydroxyalkyl, mercapto, nitro, oxo, —NR_{A}R_{B}, (NR_{A}R_{B})alkyl, (NR_{A}R_{B})carbonyl and (NR_{A}R_{B})sulfonyl.

The term “heterocyclealkyl,” as used herein, refers to a heterocycle, as defined herein, appended to the parent molecular moiety through an alkyl group, as defined herein. Representative examples of heterocyclealkyl include, but are not limited to, pyridin-3-ylmethyl and 2-pyrimidin-2-ylpropyl.

The term “heterocyclecarbonyl,” as used herein, refers to a heterocycle, as defined herein, appended to the parent molecular moiety through a carbonyl group, as defined herein. Representative examples of heterocyclecarbonyl include, but are not limited to, 1H-imidazol-1-ylcarbonyl, 4-morpholinylcarbonyl, 1-piperidinylcarbonyl and cyclopentylaminocarbonyl.

The term “hydroxy,” as used herein, refers to an —OH group.

The term “hydroxyalkyl,” as used herein, refers to one or two hydroxy groups, as defined herein, appended to the parent molecular moiety through an alkyl group, as defined herein. Representative examples of hydroxyalkyl include, but are not limited to, hydroxymethyl, 2-hydroxyethyl, 3-hydroxypropyl, 2,3-dihydroxypropyl and 2-ethyl-4-hydroxyheptyl.

The term “mercapto,” as used herein, refers to a —SH group.

The term “nitro,” as used herein, refers to a —NO₂ group.

The term “—NR_{A}R_{B},” as used herein, refers to two groups, R_{A} and R_{B}, which are appended to the parent molecular moiety through a nitrogen atom. R_{A} and R_{B} are each independently selected from hydrogen, alkyl, alkylcarbonyl, and formyl. Representative examples of —NR_{A}R_{B} include, but are not limited to, acetylamino, amino, formylamino, dimethylamino, methylamino, phenylamino, and 3-pyridinylamino.

The term “(NR_{A}R_{B})alkyl,” as used herein, refers to a —NR_{A}R_{B} group, as defined herein, appended to the parent molecular moiety through an alkyl group, as defined herein. Representative examples of (NR_{A}R_{B})alkyl include, but are not limited to, (amino)methyl, (dimethylamino)methyl and (ethylamino)methyl.

The term “(NR_{A}R_{B})carbonyl,” as used herein, refers to a —NR_{A}R_{B} group, as defined herein, appended to the parent molecular moiety through a carbonyl group, as defined herein. Representative examples of (NR_{A}R_{B})carbonyl include, but are not limited to, aminocarbonyl, dimethylaminocarbonyl and ethylaminocarbonyl.

The term “(NR_{A}R_{B})sulfonyl,” as used herein, refers to an amino group, as defined herein, appended to the parent molecular moiety through a sulfonyl group, as defined herein. Representative examples of aminosulfonyl include, but are not limited to, aminosulfonyl, dimethylaminosulfonyl and ethylaminosulfonyl.

The term “oxo,” as used herein, refers to a ═O moiety.

The term “oxy,” as used herein, refers to a —O— moiety.

The term “sulfinyl,” as used herein, refers to a —S(O)— group.

The term “sulfonyl,” as used herein, refers to a —SO₂— group.

According to the present invention, pharmaceutical compositions comprising a therapeutically effective amount of a compound of formula (I-V) in combination with a pharmaceutically acceptable carrier, are provided.

According to one embodiment of the present invention, a method of selectively modulating the effects of the histamine-3 receptors in a mammal comprising administering an effective amount of a compound of formula (I-V), is provided.

According to another embodiment of the present invention, a method of treating a disorder wherein the disorder is ameliorated by modulating the histamine-3 receptors in a mammal comprising administering an effective amount of a compound of formula (I-V), is provided.

According to still another embodiment of the present invention, a method of treating a disorder wherein the disorder is selected from the group consisting of acute myocardial infarction, asthma, cutaneous carcinoma, depression, inflammation, medullary thyroid carcinoma, melanoma, Meniere's disease, migraine, motion sickness, pain, Parkinson's disease, schizophrenia, seizures, and septic shock comprising administering an effective amount of a compound of formula (I-V), is provided.

According to another embodiment of the present invention, a method of treating a disorder wherein the disorder is Alzheimer's disease comprising administering an effective amount of a compound of formula (I-V), is provided.

According to another embodiment of the present invention, a method of treating a disorder wherein the disorder is attention-deficit hyperactivity disorder comprising administering an effective amount of a compound of formula (I-V), is provided.

According to another embodiment of the present invention, a method of treating a disorder wherein the disorder is epilepsy comprising administering an effective amount of a compound of formula (I-V), is provided.

According to another embodiment of the present invention, a method of treating a disorder wherein the disorder is narcolepsy comprising administering an effective amount of a compound of formula (I-V), is provided.

According to still another embodiment of the present invention, a method of treating a disorder wherein the disorder is obesity comprising administering an effective amount of a compound of formula (I-V), is provided.

According to still another embodiment of the present invention, a method of treating a disorder wherein the disorder is selected from the group consisting of mild cognitive impairment, deficits of memory, deficits of learning and dementia.

Representative compounds of formula (I) include, but are not limited to:

(4-fluorophenyl)(2-{2-[(2R)-2-methyl-1-pyrrolidinyl]ethyl}-1-benzofuran-5-yl)methanone;

(3-fluorophenyl)(2-{2-[(2R)-2-methyl-1-pyrrolidinyl]ethyl}-1-benzofuran-5-yl)methanone;

(2-fluorophenyl)(2-{2-[(2R)-2-methyl-1-pyrrolidinyl]ethyl}-1-benzofuran-5-yl)methanone;

(3-chlorophenyl)(2-{2-[(2R)-2-methyl-1-pyrrolidinyl]ethyl}-1-benzofuran-5-yl)methanone;

(4-chlorophenyl)(2-{2-[(2R)-2-methyl-1-pyrrolidinyl]ethyl}-1-benzofuran-5-yl)methanone;

(4-methoxyphenyl)(2-{2-[(2R)-2-methyl-1-pyrrolidinyl]ethyl}-1-benzofuran-5-yl)methanone;

(4-fluoro-3-methylphenyl)(2-{2-[(2R)-2-methyl-1-pyrrolidinyl]ethyl}-1-benzofuran-5-yl)methanone;

cyclopropyl(2-{2-[(2R)-2-methyl-1-pyrrolidinyl]ethyl}-1-benzofuran-5-yl)methanone;

3-ethyl-1-(2-{2-[(2R)-2-methyl-1-pyrrolidinyl]ethyl}-1-benzofuran-5-yl)-1-pentanone;

(4-chloro-3-methylphenyl)(2-{2-[(2R)-2-methyl-1-pyrrolidinyl]ethyl}-1-benzofuran-5-yl)methanone;

(2-{2-[(2R)-2-methyl- 1-pyrrolidinyl]ethyl}-1-benzofuran-5-yl)[4-(methylthio)phenyl]methanone;

[4-(dimethylamino)phenyl](2-{2-[(2R)-2-methyl-1-pyrrolidinyl]ethyl}-1-benzofuran-5-yl)methanone;

(4-methylphenyl)(2-{2-[(2R)-2-methyl-1-pyrrolidinyl]ethyl}-1-benzofuran-5-yl)methanone;

(3,5-difluorophenyl)(2-{2-[(2R)-2-methyl-1-pyrrolidinyl]ethyl}-1-benzofuran-5-yl)methanone;

(2-methoxyphenyl)(2-{2-[(2R)-2-methyl-1-pyrrolidinyl]ethyl}-1-benzofuran-5-yl)methanone;

(3-methoxyphenyl)(2-{2-[(2R)-2-methyl-1-pyrrolidinyl]ethyl}-1-benzofuran-5-yl)methanone; and

(2-{2-[(2R)-2-methyl-1-pyrrolidinyl]ethyl}-1-benzofuran-5-yl)(phenyl)methanone.

Representative compounds of formula (II) include, but are not limited to:

4-{2-[2-(diethylamino)ethyl]-1-benzofuran-5-yl}benzonitrile;

4-(2-{2-[tert-butyl(methyl)amino]ethyl}-1-benzofuran-5-yl)benzonitrile;

4-(2-{2-[isopropyl(methyl)amino]ethyl}-1-benzofuran-5-yl)benzonitrile;

4-(2-{2-[isobutyl(methyl)amino]ethyl}-1-benzofuran-5-yl)benzonitrile;

4-(2-{2-[ethyl(isopropyl)amino]ethyl}-1-benzofuran-5-yl)benzonitrile; and

4-(2-{2-[ethyl(propyl)amino]ethyl}-1-benzofuran-5-yl)benzonitrile;

N,N-diethyl-N-(2-{5-[4-(4-morpholinylcarbonyl)phenyl]-1-benzofuran-2-yl}ethyl)amine;

N-(tert-butyl)-N-methyl-N-(2-{5-[4-(4-morpholinylcarbonyl)phenyl]-1-benzofuran-2-yl}ethyl)amine;

N-isopropyl-N-methyl-N-(2-{5-[4-(4-morpholinylcarbonyl)phenyl]-1-benzofuran-2-yl}ethyl)amine;

N-isobutyl-N-methyl-N-(2-{5-[4-(4-morpholinylcarbonyl)phenyl]-1-benzofuran-2-yl}ethyl)amine;

N-ethyl-N-isopropyl-N-(2-{5-[4-(4-morpholinylcarbonyl)phenyl]-1-benzofuran-2-yl}ethyl)amine;

N,N-dimethyl-N-(2-{5-[4-(4-morpholinylcarbonyl)phenyl]-1-benzofuran-2-yl}ethyl)amine;

N-ethyl-N-(2-{5-[4-(4-morpholinylcarbonyl)phenyl]-1-benzofuran-2-yl}ethyl)-N-propylamine;

4-[(6-{2-[2-(N,N-diethyl)ethyl]-1-benzofuran-5-yl}-3-pyridinyl)carbonyl]morpholine;

N-(tert-butyl)-N-methyl-N-(2-{5-[5-(4-morpholinylcarbonyl)-2-pyridinyl]-1-benzofuran-2-yl}ethyl)amine;

N-isobutyl-N-methyl-N-(2-{5-[5-(4-morpholinylcarbonyl)-2-pyridinyl]-1-benzofuran-2-yl}ethyl)amine;

N-isopropyl-N-methyl-N-(2-{5-[5-(4-morpholinylcarbonyl)-2-pyridinyl]-1-benzofuran-2-yl}ethyl)amine;

N-ethyl-N-isopropyl-N-(2-{5-[5-(4-morpholinylcarbonyl)-2-pyridinyl]-1-benzofuran-2-yl}ethyl)amine;

N,N-dimethyl-N-(2-{5-[5-(4-morpholinylcarbonyl)-2-pyridinyl]-1-benzofuran-2-yl}ethyl)amine;

N-ethyl-N-propyl-N-(2-{5-[5-(4-morpholinylcarbonyl)-2-pyridinyl]-1-benzofuran-2-yl}ethyl)amine;

N-allyl-N-(2-{5-[5-(4-morpholinylcarbonyl)-2-pyridinyl]-1-benzofuran-2-yl}ethyl)amine;

3-[(2-{5-[5-(4-morpholinylcarbonyl)-2-pyridinyl]-1-benzofuran-2-yl}ethyl)amino]-1-propanol;

N-(2-{5-[5-(4-morpholinylcarbonyl)-2-pyridinyl]-1-benzofuran-2-yl}ethyl)-N-propylamine;

4-{2-[2-(1-pyrrolidinyl)ethyl]-1-benzofuran-5-yl}benzonitrile;

4-{2-[2-(2-methyl-1-pyrrolidinyl)ethyl]-1-benzofuran-5-yl}benzonitrile;

4-{2-[2-(1-piperidinyl)ethyl]-1-benzofuran-5-yl}benzonitrile;

4-{2-[2-(2-methyl-1-piperidinyl)ethyl]-1-benzofuran-5-yl}benzonitrile;

4-(2-{2-[(3R)-3-hydroxypyrrolidinyl]ethyl}-1-benzofuran-5-yl)benzonitrile;

4-{2-[2-(1H-imidazol-1-yl)ethyl]-1-benzofuran-5-yl}benzonitrile;

4-(2-{2-[(3S)-3-(dimethylamino)pyrrolidinyl]ethyl}-1-benzofuran-5-yl)benzonitrile;

4-(2-{2-[(2S)-2-(hydroxymethyl)pyrrolidinyl]ethyl}-1-benzofuran-5-yl)benzonitrile;

4-(2-{2-[(2R,6S)-2,6-dimethylpiperidinyl]ethyl}-1-benzofuran-5-yl)benzonitrile;

4-(2-{2-[(2R,5R)-2,5-dimethylpyrrolidinyl]ethyl}-1-benzofuran-5-yl)benzonitrile;

4-{2-[2-(1-azepanyl)ethyl]-1-benzofuran-5-yl}benzonitrile;

4-{2-[2-(4-methyl-1-piperidinyl)ethyl]-1-benzofuran-5-yl}benzonitrile;

4-(2-{2-[2-pyrrolidine methyl carboxylate]ethyl}-1-benzofuran-5-yl)benzonitrile;

4-{2-[2-(3,6-dihydro-1(2H)-pyridinyl)ethyl]-1-benzofuran-5-yl}benzonitrile;

4-(2-{2-[(2R)-2-(hydroxymethyl)pyrrolidinyl]ethyl}-1-benzofuran-5-yl)benzonitrile;

4-(2-{2-[(3R)-(dimethylamino)pyrrolidinyl]ethyl}-1-benzofuran-5-yl)benzonitrile;

4-(2-{2-[1-(2R)-2-methylpyrrolidinyl]ethyl}-1-benzofuran-5-yl)benzonitrile;

4-(4-{2-[2-(2-methyl-1-pyrrolidinyl)ethyl]-1-benzofuran-5-yl}benzoyl)morpholine;

4-(4-{2-[2-(1-piperidinyl)ethyl]-1-benzofuran-5-yl}benzoyl)morpholine;

4-(4-{2-[2-(2-methyl-1-piperidinyl)ethyl]-1-benzofuran-5-yl}benzoyl)morpholine;

(3R)-1-(2-{5-[4-(4-morpholinylcarbonyl)phenyl]-1-benzofuran-2-yl}ethyl-3-pyrrolidinol;

4-[4-(2-{2-[(2R,5R)-2,5-dimethylpyrrolidinyl]ethyl}-1-benzofuran-5-yl)benzoyl]morpholine;

4-[4-(2-{2-[(2R,6S)-2,6-dimethylpiperidinyl]ethyl}-1-benzofuran-5-yl)benzoyl]morpholine;

4-(4-{2-[2-(azepinyl)ethyl]-1-benzofuran-5-yl}benzoyl)morpholine;

4-(4-{2-[2-(4-methyl-1-piperidinyl)ethyl]-1-benzofuran-5-yl}benzoyl)morpholine;

4-(4-{2-[2-(4-morpholine)ethyl]-1-benzofuran-5-yl}benzoyl)morpholine;

4-(4-{2-[2-(3,6-dihydro-1(2H)-pyridinyl)ethyl]-1-benzofuran-5-yl}benzoyl)morpholine;

4-(4-{2-[2-(2S)-pyrrolidinylmethanol)ethyl]-1-benzofuran-5-yl}benzoyl)morpholine;

4-[(6-{2-[2-(1-pyrrolidinyl)ethyl]-1-benzofuran-5-yl}-3-pyridinyl)carbonyl]morpholine;

4-{[6-(2-{2-[(2R)-methylpyrrolidinyl]ethyl}-1-benzofuran-5-yl)-3-pyridinyl]carbonyl}morpholine;

4-[(6-{2-[2-(1-piperidinyl)ethyl]-1-benzofuran-5-yl}-3-pyridinyl)carbonyl]morpholine;

(3R)-1-(2-{5-[5-(4-morpholinylcarbonyl)-2-pyridinyl]-1-benzofuran-2-yl}ethyl)-3-pyrrolidinol;

4-{[6-(2-{2-[(2R,5R)-2,5-dimethylpyrrolidinyl]ethyl}-1-benzofuran-5-yl)-3-pyridinyl]carbonyl}morpholine;

4-{[6-(2-{2-[(2R,6S)-2,6-dimethylpiperidinyl]ethyl}-1-benzofuran-5-yl)-3-pyridinyl]carbonyl}morpholine;

4-{[6-(2-{2-[1-azepanyl]ethyl}-1-benzofuran-5-yl)-3-pyridinyl]carbonyl}morpholine;

4-[(6-{2-[2-(4-methyl-1-piperidinyl)ethyl]-1-benzofuran-5-yl}-3-pyridinyl)carbonyl]morpholine;

4-[(6-{2-[2-(4-morpholinyl)ethyl]-1-benzofuran-5-yl}-3-pyridinyl)carbonyl]morpholine;

8-(2-{5-[5-(4-morpholinylcarbonyl)-2-pyridinyl]-1-benzofuran-2-yl}ethyl)-1,4-dioxa-8-azaspiro[4.5]decane;

5-(2-{5-[5-(4-morpholinylcarbonyl)-2-pyridinyl]-1-benzofuran-2-yl}ethyl)-2-oxa-5-azabicyclo[2.2.1]heptane; and

(2S)-1-(2-{5-[5-(4-morpholinylcarbonyl)-2-pyridinyl]-1-benzofuran-2-yl}ethyl)-2-pyrrolidinol.

The following additional compounds, representative of formula (II), may be prepared by one skilled in the art using known synthetic methodology or by using synthetic methodology described in the Schemes and Examples contained herein:

4-(4-{2-[2-(1-pyrrolidinyl)ethyl]-1-benzofuran-5-yl}benzoyl)morpholine;

4-{4-methyl-2-oxo-3-[2-(1-pyrrolidinyl)ethyl]-2H-chromen-6-yl}benzonitrile;

4-{4-methyl-3-[2-(2-methyl-1-pyrrolidinyl)ethyl]-2-oxo-2H-chromen-6-yl}benzonitrile;

4-{4-methyl-3-[2-(2-methyl-1-piperidinyl)ethyl]-2-oxo-2H-chromen-6-yl}benzonitrile;

4-methyl-6-[4-(4-morpholinylcarbonyl)phenyl]-3-[2-(1-pyrrolidinyl)ethyl]-2H-chromen-2-one;

4-methyl-3-[2-(2-methyl-1-pyrrolidinyl)ethyl]-6-[4-(4-morpholinylcarbonyl)phenyl]-2H-chromen-2-one;

4-methyl-3-[2-(2-methyl- 1-piperidinyl)ethyl]-6-[4-(4-morpholinylcarbonyl)phenyl]-2H-chromen-2-one;

4-(2-{2-[1-(2S)-2-methylpyrrolidinyl]ethyl}-1-benzofuran-5-yl)benzonitrile; and

4-(2-{2-[1-(2-methylpyrrolidinyl]ethyl}-1-benzofuran-5-yl)benzonitrile.

Representative compounds of formula (III) include, but are not limited to:

4-{3-[2-(diethylamino)ethyl]-4-methyl-2-oxo-2H-chromen-7-yl}benzonitrile;

4-{4-methyl-2-oxo-3-[2-(1-pyrrolidinyl)ethyl]-2H-chromen-7-yl}benzonitrile; and

4-{4-methyl-2-oxo-3-[2-(1-piperidinyl)ethyl]-2H-chromen-7-yl}benzonitrile.

The following additional compounds, representative of formula (III), may be prepared by one skilled in the art using known synthetic methodology or by using synthetic methodology described in the Schemes and Examples contained herein:

4-{2-[2-(1-pyrrolidinyl)ethyl]-1-benzofuran-6-yl}benzonitrile;

4-{2-[2-(2-methyl-1-pyrrolidinyl)ethyl]-1-benzofuran-6-yl}benzonitrile;

4-{2-[2-(2-methyl-1-piperidinyl)ethyl]-1-benzofuran-6-yl}benzonitrile;

4-(4-{2-[2-(1-pyrrolidinyl)ethyl]-1-benzofuran-6-yl}benzoyl)morpholine;

4-(4-{2-[2-(2-methyl-1-pyrrolidinyl)ethyl]-1-benzofuran-6-yl}benzoyl)morpholine;

4-(4-{2-[2-(2-methyl-1-piperidinyl)ethyl]-1-benzofuran-6-yl}benzoyl)morpholine;

4-{4-methyl-3-[2-(2-methyl-1-pyrrolidinyl)ethyl]-2-oxo-2H-chromen-7-yl}benzonitrile;

4-{4-methyl-3-[2-(2-methyl-1-piperidinyl)ethyl]-2-oxo-2H-chromen-7-yl}benzonitrile;

4-methyl-7-[4-(4-morpholinylcarbonyl)phenyl]-3-[2-(1-pyrrolidinyl)ethyl]-2H-chromen-2-one;

4-methyl-3-[2-(2-methyl-1-pyrrolidinyl)ethyl]-7-[4-(4-morpholinylcarbonyl)phenyl]-2H-chromen-2-one; and

4-methyl-3-[2-(2-methyl-1-piperidinyl)ethyl]-7-[4-(4-morpholinylcarbonyl)phenyl]-2H-chromen-2-one.

Representative compounds of formula (IV) include, but are not limited to:

4-(2-{2-[(2R)-2-methylpyrrolidinyl]ethyl}-2,3-dihydro-1-benzofuran-5-yl)benzonitrile.

The following additional compounds, representative of formula (IV), may be prepared by one skilled in the art using known synthetic methodology or by using synthetic methodology described in the Schemes and Examples contained herein.

4-{2-[2-(1-pyrrolidinyl)ethyl]-2,3-dihydro-1-benzofuran-5-yl}benzonitrile;

4-{2-[2-(2-methyl-1-piperidinyl)ethyl]-2,3-dihydro-1-benzofuran-5-yl}benzonitrile;

4-(4-{2-[2-(1-pyrrolidinyl)ethyl]-2,3-dihydro-1-benzofuran-5-yl}benzoyl)morpholine;

4-(4-{2-[2-(2-methyl-1-pyrrolidinyl)ethyl]-2,3-dihydro-1-benzofuran-5-yl}benzoyl)morpholine; and

4-(4-{2-[2-(2-methyl-1-piperidinyl)ethyl]-2,3-dihydro-1-benzofuran-5-yl}benzoyl)morpholine.

The following additional compounds, representative of formula (IV), may be prepared by one skilled in the art using known synthetic methodology or by using synthetic methodology described in the Schemes and Examples contained herein:

4-{2-[3-(1-pyrrolidinyl)propyl]-2,3-dihydro-1-benzofuran-6-yl}benzonitrile;

4-{2-[3-(2-methyl-1-pyrrolidinyl)propyl]-2,3-dihydro-1-benzofuran-6-yl}benzonitrile;

4-{2-[3-(2-methyl-1-piperidinyl)propyl]-2,3-dihydro-1-benzofuran-6-yl}benzonitrile;

4-(4-{2-[3-(1-pyrrolidinyl)propyl]-2,3-dihydro-1-benzofuran-6-yl}benzoyl)morpholine;

4-(4-{2-[3-(2-methyl-1-pyrrolidinyl)propyl]-2,3-dihydro-1-benzofuran-6-yl}benzoyl)morpholine; and

4-(4-{2-[3-(2-methyl-1-piperidinyl)propyl]-2,3-dihydro-1-benzofuran-6-yl}benzoyl)morpholine.

### Determination of Biological Activity

To determine the effectiveness of representative compounds of this invention as histamine-3 receptor ligands (H₃ receptor ligands), the following tests were conducted according to methods previously described (European Journal of Pharmacology, 188:219-227 (1990); Journal of Pharmacology and Experimental Therapeutics, 275: 598-604 (1995); Journal of Pharmacology and Experimental Therapeutics, 276:1009-1015 (1996); and Biochemical Pharmacology, 22: 3099-3108 (1973)).

Briefly, male Sprague-Dawley rat brain cortices were homogenized (1 g tissue/10 mL buffer) in 50 mM Tris-HCl/5 mM EDTA containing protease inhibitor cocktail (Calbiochem) using a polytron set at 20,500 rpm. Homogenates were centrifuged for 20 minutes at 40,000×g. The supernatant was decanted, and pellets were weighed. The pellet was resuspended by polytron homogenization in 40 mL 50 mM Tris-HCl/5 mM EDTA with protease inhibitors and centrifuged for 20 minutes at 40,000×g. The membrane pellet was resuspended in 6.25 volumes (per gram wet weight of pellet) of 50 mM Tris-HCl/5 mM EDTA with protease inhibitors and aliquots flash frozen in liquid N₂ and stored at −70° C. until used in assays. Rat cortical membranes (12 mg wet weight/tube) were incubated with (³H)-N-α-methylhistamine (˜0.6 nM) with or without H₃ receptor antagonists in a total incubation volume of 0.5 mL of 50 mM Tris-HCl/5 mM EDTA (pH 7.7). Test compounds were dissolved in DMSO to provide a 20 mM solution, serially diluted and then added to the incubation mixtures prior to initiating the incubation assay by addition of the membranes. Thioperamide (3 μM) was used to determine nonspecific binding. Binding incubations were conducted for 30 minutes at 25° C. and terminated by addition of 2 mL of ice cold 50 mM Tris-HCl (pH 7.7) and filtration through 0.3% polyethylenimine-soaked Unifilter plates (Packard). These filters were washed 4 additional times with 2 mL of ice-cold 50 mM Tris-HCl and dried for 1 hour. Radioactivity was determined using liquid scintillation counting techniques. Results were analyzed by Hill transformation and Ki values were determined using the Cheng-Prusoff equation.

TABLE 1

| Example Number | Ki (nM) |
|---|---|
| 1 | 4.44 |
| 2 | 46.8 |
| 3 | 7.45 |
| 4 | 58.8 |
| 5 | 49.4 |
| 6 | 44.9 |
| 7 | 94.9 |
| 8 | 1995 |
| 9 | 136 |
| 10 | 22.9 |
| 11 | 19.3 |
| 12 | 38.4 |
| 13 | 78.4 |
| 14 | 25.1 |
| 15 | 1000 |
| 16 | 92.2 |
| 17 | 165 |
| 18 | 60.5 |
| 19 | 77.7 |
| 20 | 180 |
| 21 | 44.4 |
| 22 | 69.2 |
| 23 | 1.97 |
| 24 | 11.7 |
| 25 | 14.4 |
| 26 | 27.2 |
| 27 | 55.4 |
| 28 | 9.24 |
| 29 | 8.46 |
| 30 | 13.7 |
| 31 | 24.6 |
| 32 | 265 |
| 33 | 32.3 |
| 34 | 6.89 |
| 35 | 67.9 |
| 36 | 52.1 |
| 37 | 248 |
| 38 | 26.0 |
| 39 | 148 |
| 40 | 32.2 |
| 41 | 51.5 |
| 42 | 41.8 |
| 43 | 14.6 |
| 44 | 17.2 |
| 45 | 1.61 |
| 46 | 18.5 |
| 47 | 59.8 |
| 48 | 30.8 |
| 49 | 14.4 |
| 50 | 37.1 |
| 51 | 3.07 |
| 52 | 162 |
| 53 | 242 |
| 54 | 197 |
| 55 | 575 |
| 56 | 105 |
| 57 | 115 |
| 58 | 133 |
| 59 | 79.1 |
| 60 | 1000 |
| 61 | 143 |
| 62 | 112 |
| 63 | 1000 |
| 64 | 1000 |
| 65 | 596 |
| 66 | 90.4 |
| 68 | 2.2 |
| 69 | 0.6 |
| 70 | 2.1 |
| 71 | 2.0 |
| 72 | 2.7 |
| 73 | 2.9 |
| 74 | 3.5 |
| 75 | 9.9 |
| 76 | 4.0 |
| 77 | 6.0 |
| 78 | 8.2 |
| 79 | 3.8 |
| 80 | 1.4 |
| 81 | 1.4 |
| 82 | 1.6 |
| 83 | 1.1 |
| 84 | 2.8 |

As shown by the data in Table 1, the compounds of the present invention bind to the histamine-3 receptors and therefore may have utility in the treatment of diseases or conditions ameliorated with histamine-3 receptor ligands.

Compounds of the present invention are histamine-3 receptor ligands that modulate function of the histamine-3 receptor by antagonizing the activity of the receptor. These compounds may be inverse agonists that inhibit the basal activity of the receptor or they may be antagonists that completely block the action of receptor-activating agonists. These compounds may also be partial agonists that partially block or partially activate the histamine-3 receptor receptor or they may be agonists that activate the receptor.

### Abbreviations

Abbreviations which have been used in the descriptions of the schemes and the examples that follow are: Ac for acetyl; DCM for dichloromethane; DMF for N,N-dimethylformamide; DMSO for dimethylsulfoxide; HPLC for high pressure liquid chromatography; Me for methyl; TFA for trifluoroacetic acid; TosCl for p-toluenesufonyl chloride; TBDMS for tert-butyldimethylsilyl; THF for tetrahydrofuran; TMEDA for N,N,N′, N′-tetramethylethylenediamine; and p-TSA for para-toluenesulfonic acid.

### Preparations of Compounds of The Present Invention

The compounds and processes of the present invention will be better understood in connection with the following synthetic schemes and methods which illustrate a means by which the compounds can be prepared.

The compounds of this invention can be prepared by a variety of synthetic procedures. Representative procedures are shown in, but are not limited to, Schemes 1-12.

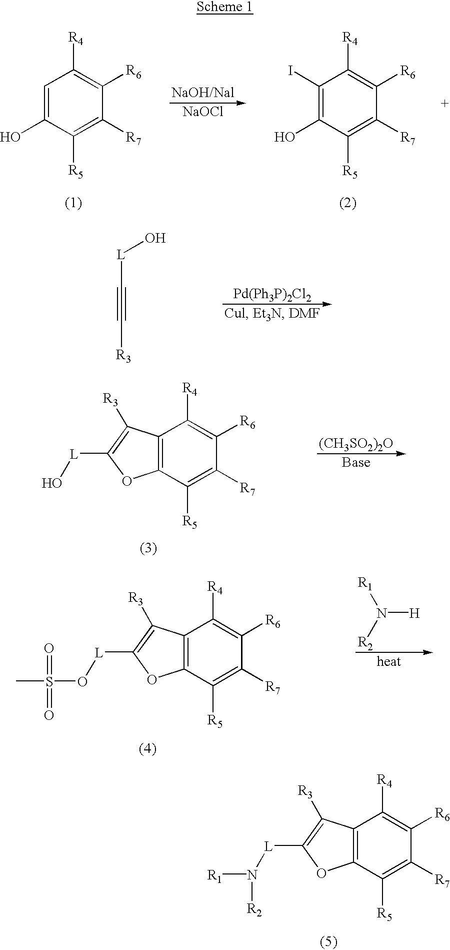

Benzofurans of general formula (5), wherein L, R₁, R₂, R₃, R₄, R₅, R₆ and R₇ are as defined in formula (I), may be prepared as described in Scheme I. Phenols of general formula (1) may be treated with sodium hypochlorite, sodium iodide and sodium hydroxide in a solvent such as methanol to provide iodides of general formula (2). Iodides of general formula (2) may be treated with substituted propargyl alcohols, dichlorobis(triphenylphosphine)palladium, copper iodide, a base such as triethylamine in a solvent such as DMF with heat to provide benzofurans of general formula (3). Alcohols of general formula (3) may be treated with methanesulfonyl chloride or methanesulfonyl anhydride, a base such as triethylamine, diisopropylethylamine or N-methylmorpholine in a solvent such as dichloromethane or THF to provide mesylates of general formula (4). Mesylates of general formula (4) may be treated with secondary or primary amines in solvents such as DMF or THF with heat to provide amines of general formula (5). Alternatively mesylates of general formula (4) may be treated with secondary or primary amine hydrochlorides in the presence of a base such as triethylamine, diisopropylethylamime or N-methylmorpholine in a solvent such as DMF or THF with heat to provide benzofurans of general formula (5).

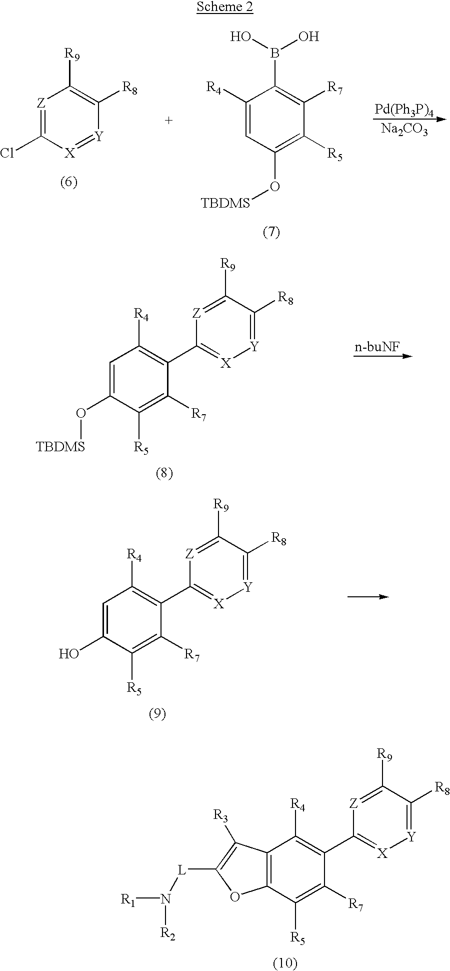

Benzofurans of general formula (10), wherein L, R₁, R₂, R₃, R₄, R₅, R₇, R₈, R₉, X, Y and Z are as defined in formula (II), may be prepared as described in Scheme 2. Chlorides of general formula (6) may be treated with boronic acids of general formula (7), tetrakis(triphenylphosphine)palladium, a base such as aqueous sodium carbonate in a solvent such as toluene with heat to provide tert-butyldimethylsilyl protected alcohols of general formula (8). Protected alcohols of general formula (8) may be treated with tetrabutylammonium fluoride in a solvent such as THF to provide alcohols of general formula (9). Alcohols of general formula (9) may be treated using conditions as described in Scheme 1 to provide benzofurans of general formula (10).

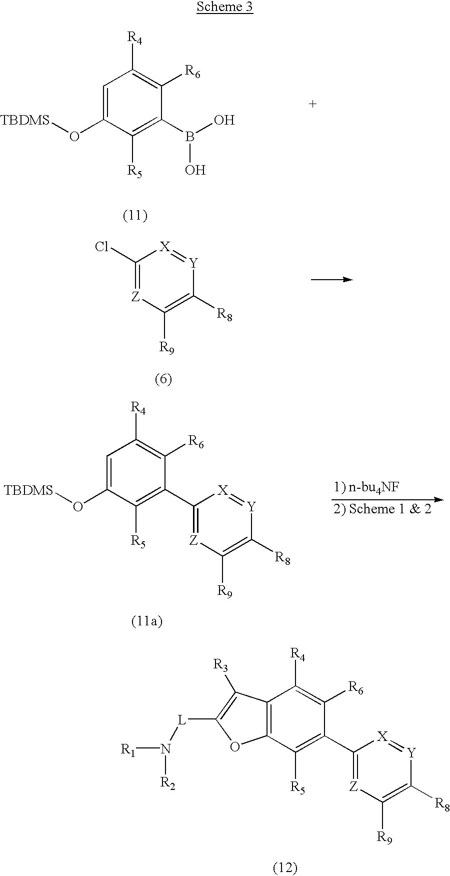

Benzofurans of general formula (12), wherein L, R₁, R₂, R₃, R₄, R₅, R₆, X, Y, Z, R₈, and R₉ are as defined in formula (III), may be prepared as described in Scheme 3. Boronic acids of general formula (11) may be treated with chlorides as described in Scheme 2 to provide compounds of general formula (11a). Compounds of general formula (11a) may be treated with tetra-butylammonium fluoride and then as described in Scheme 1 and Scheme 2 to provide benzofurans of general formula (12).

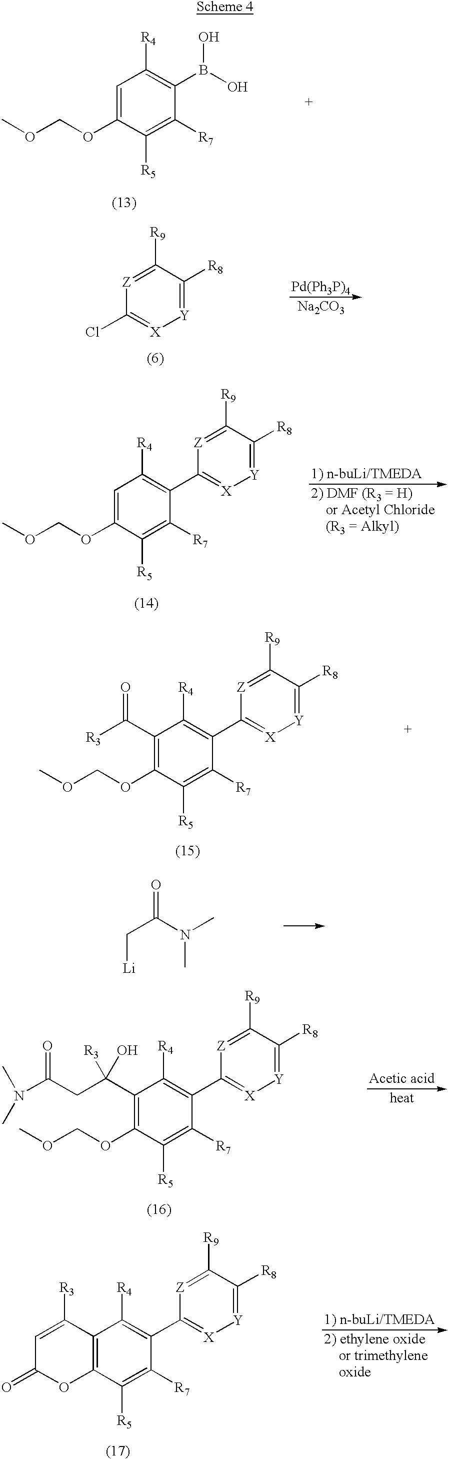

Chromenes of general formula (19), wherein L, R₁, R₂, R₃, P,, R₅, R₇, R₈, R₉, X, Y and Z are as defined by formula (II), may be prepared as described in Scheme 4. Boronic acids of general formula (13) may be treated with chlorides of general formula (6), tetrakis(triphenylphosphine)palladium, a base such as aqueous sodium carbonate in a solvent such as toluene with heat to provide compounds of general formula (14). Compounds of general formula (14) may be treated with n-butyl lithium, N, N, N′, N′-tetramethylethylenediamine followed by DMF or acetyl chloride to provide compounds of general formula (15) which may be treated with [2-(dimethylamino)-2-oxoethyl]lithium in a solvent such as THF to provide compounds of general formula (16). Compounds of general formula (16) may be treated with acetic acid with heat to provide chromenes of general formula (17). Chromenes of general formula (17) may be treated with butyl lithium, N,N, N′,N′-tetramethylethylenediamine followed by ethylene oxide or trimethylene oxide to provide alcohols of general formula (18). Alternatively (17) may be treated with butyl lithium, N, N, N′, N′-tetramethylethylenediamine and (2-bromoethoxy) tert-butyldimethylsilane followed by tetrabutylammonium fluoride deprotection to provide alcohols of general formula (18). Alcohols of general formula (18) may be converted to the respective mesylate and further reacted with amines as described in scheme 1 to provide chromenes of general formula (19).

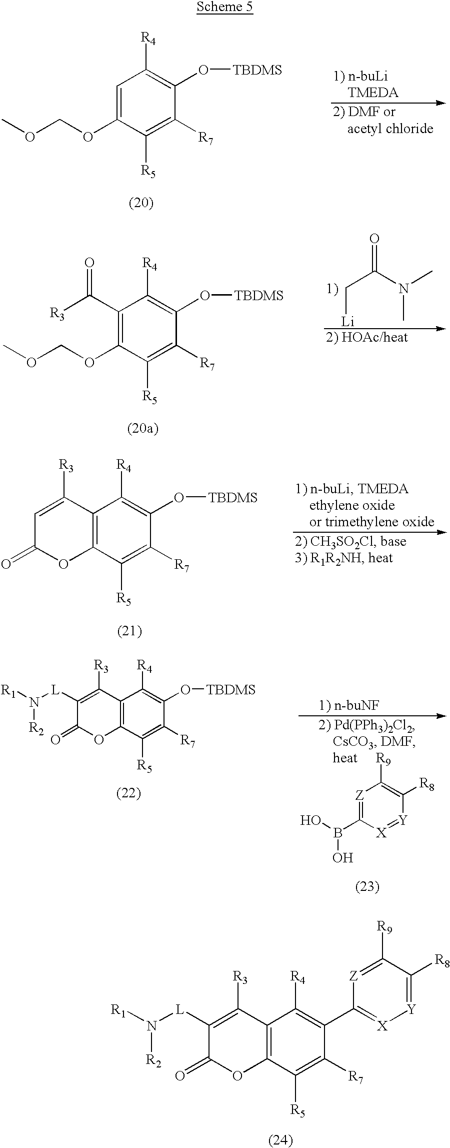

Alternatively chromenes of general formula (24) wherein L, R₁, R₂, R₃, R₄, R₅, R₇, R₈ and R₉ are as defined in formula (II), may be prepared as described in scheme 5. Compound of general structure (21) may be prepared from Scheme 4 by substituting compound of general structure (20) for compound of general formula (14). Compound of general formula (20) may be treated with n-butyl lithium followed by DMF or acetyl chloride to provide compounds of general formula (20a). Compounds of general formula (20a) may be treated with [2-(dimethylamino)-2-oxoethyl]lithium followed by acetic acid and heat to provide chromenes of general formula (21). Chromenes of general formula (21) may be treated with n-butyl lithium, N,N,N′,N′-tetramethylethylenediamine followed by ethylene oxide or trimethylene oxide to provide alcohols which may be treated with methanesulfonyl chloride, a base such as triethylamine, diisopropylethylamine or N-methylmorpholine in a solvent such as dichloromethane or THF to provide mesylates which may be treated with secondary or primary amines in solvents such as DMF or THF with heat to provide amines of general formula (22). Amines of general formula (22) may be treated with tetrabutylammonium fluoride followed by treatment with boronic acids of general formula (23), dichlorobis(triphenylphosphine)palladium, cesium carbonate in a solvent such as DMF with heat to provide compound of general formula (24).

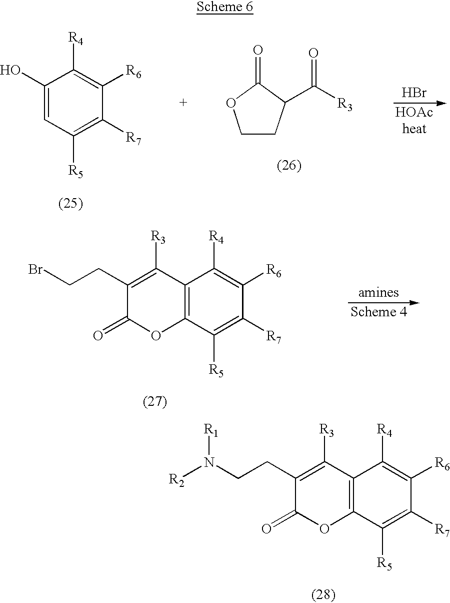

Alternatively chromenes of general structure (28), wherein R₁, R₂, R₃, R₄, R₅, R₆ and R₇ are as defined by formula (III), may be prepared as described in Scheme 6. Phenols of general formula (25) may be treated with compounds of general formula (26) and hydrobromic acid in acetic acid with heat to provide compounds of general formula (27), which may be treated with amines as described in Scheme 4 to provide chromenes of general formula (28).

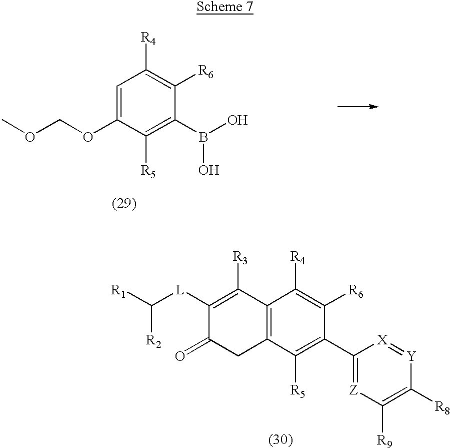

Chromenes of general formula (30) wherein L, R₁, R₂, R₃, R₄, R₅, R₆, R₈, R₉, X, Y and Z are as defined in formula (III), may be prepared as described in Scheme 7. Boronic acids of general formula (29) may be treated as described in Scheme 4 to provide Chromenes of general formula (30).

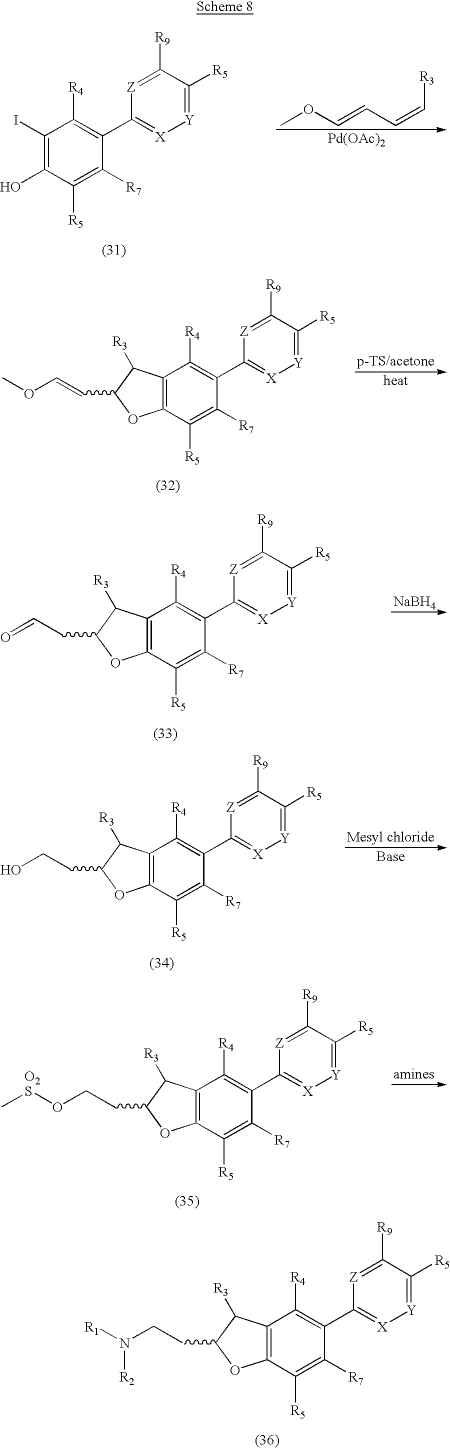

Compounds of general formula (36) wherein L, R₁, R₂, R₃, R₄, R₅, R₇, R₈, R₉, X, Y, and Z are as defined in formula (IV), may be prepared as described in Scheme 8. Iodides of general structure (31) may be treated with 5-substituted-1-methoxypentadienes and palladium acetate with heat to provide dihydrofurans of general structure (32), which may be treated with para-toluenesulfonic acid in acetone with heat to provide compounds of general formula (33). The reduction of compound of general structure (33) using sodium borohydride in solvents such as methanol may provide alcohols of general formula (34). Alcohols of general formula (34) may be treated with methanesulfonyl chloride with bases such as triethylamine, diisopropylethylamine or N-methylmorpholine in solvents such as dichloromethane or THF to provide mesylates of general formula (35). Mesylates of general formula (35) may be treated with secondary or primary amines in a solvent such as DMF or THF with heat to provide amines of general formula (36).

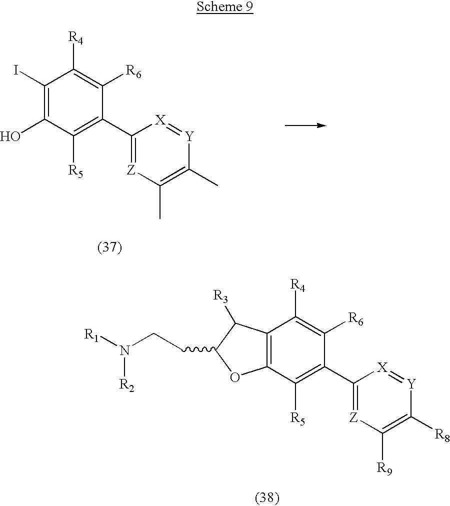

Dihydrofurans of general formula (38) wherein R₁, R₂, R₃, R₄, R₅, R₆, R₈, R₉, X, Y and Z are as defined in formula (IV), may be prepared as described in Scheme 9. Iodides of general formula (37) may be treated as described in Scheme 8 to provide dihydrofurans of general formula (38).

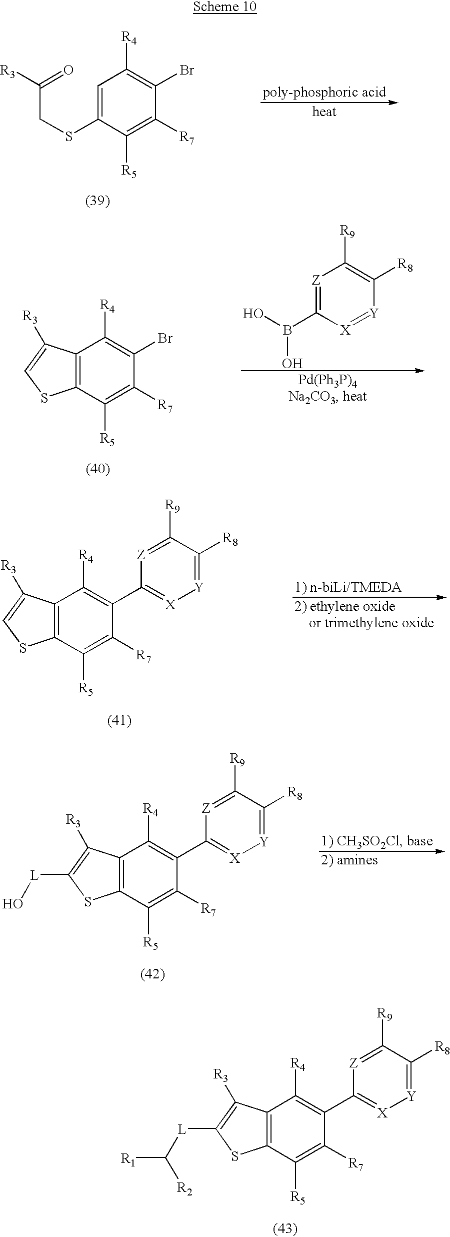

Benzothiophenes of general formula (43) wherein L, R₁, R₂, R₃, R₄, R₅, R₇, R₈, R₉, X, Y and Z are defined in formula (I), may be prepared as described in Scheme 10. Compounds of general formula (39) may be treated with poly-phosphoric acid with heat to provide benzothiophenes of general formula (40). Benzothiophenes of general formula (40) may be treated with boronic acids, tetrakis(triphenylphosphine)palladium, a base such as aqueous sodium carbonate in a solvent such as toluene with heat to provide compounds of general formula (41). Compounds of general formula (41) may be treated with n-butyl lithium, N,N, N′,N′-tetramethylethylenediamine followed by ethylene oxide to provide alcohols of general formula (42). Alcohols of general formula (42) may be converted to the mesylate and then further treated with amines as described in Scheme 1 to provide benzothiophenes of general formula (43).

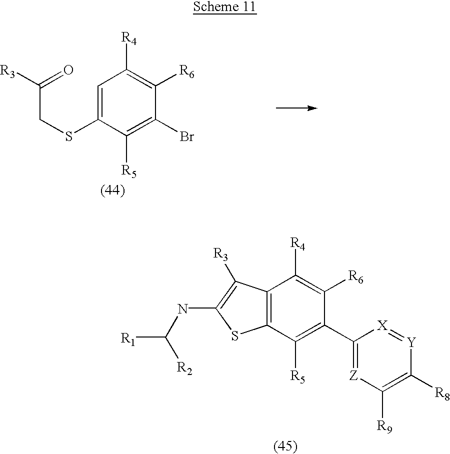

Benzothiophenes of general formula (45) wherein R₁, R₂, R₃, R₄, R₅, R₆, R₈, R₉ and X, Y and Z are defined in formula (I), may be prepared as described in Scheme 11. Compounds of general formula (44) may be processed as described in Scheme 10 to provide compounds of general formula (45).

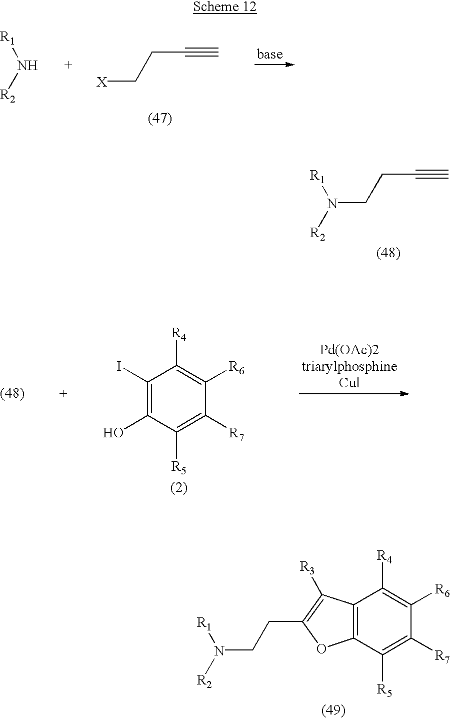

Benzofurans of general formula (49) wherein R₁, R₂, R₃, R₄, R₅, R₆, and R₇ are as defined in formula (I), may be prepared as described in Scheme 12. Primary and secondary amines may be treated with a base such as potassium carbonate (325 mesh powdered) and an alkyne of general formula (47) wherein X=Cl, Br, I, OS(O)₂CH₃ or OTs to provide alkynes of general formula (48). Alkynes of general formula (48) may be treated with a phenol of general formula (2), a palladium catalyst such as palladium(II) acetate, a triarylphoshine such as triphenylphoshine or tris(4-methylphenyl)phosphine, copper iodide, and a base such as diisopropylamine in a solvent such as acetonitrile followed by heat to provide benzofurans of general formula (49).

The compounds and processes of the present invention will be better understood by reference to the following examples, which are intended as an illustration of and not a limitation upon the scope of the invention.

### EXAMPLE 1

### 4-(2-{2-[(2R)-2-methylpyrrolidinyl]ethyl}-1-benzofuran-5-yl)benzonitrile

### Example 1A

### 4′-hydroxy-3′-iodo[1,1′-biphenyl]-4-carbonitrile

To a solution of 4-hydroxy-4′-cyanobiphenyl (6.00 g, 30.8 mmol), sodium iodide (4.61 g, 30.8 mmol) and sodium hydroxide (1.23 g, 30.8 mmol) in methanol (90 mL) at 0° C. was added an aqueous solution of sodium hypochlorite (47 mL of 5.25% Clorox™, 2.29 g, 30.8 mmol) over 45 minutes. The mixture was stirred cold for 1 hour, warmed to ambient temperature and diluted with sodium thiosulfate solution (10 mL), water (80 mL) and adjusted to a pH of 7 by addition of sodium dihydrogen phosphate. The mixture was extracted with dichloromethane (2×90 mL). The combined organic extracts were dried (Na₂SO₄), filtered and concentrated under reduced pressure to give a white powder. The solid was crystallized from dichloroethane/hexane and chromatographed on silica with dichloromethane to give the titled compound (5.19 g, 53%). MS (DCI) m/z 339[M+NH4⁺]⁺;

### Example 1B

### 4-[2-(2-hydroxyethyl)-1-benzofuran-5-yl]benzonitrile

To a solution of Example 1A (5.19 g, 16.2 mmol), triethylamine (5.60 mL, 40.4 mmol) and 3-butyn-1-ol (1.90 g, 27.2 mmol) in dimethylformamide (13 mL) at 20° C. was added cuprous iodide (0.46 g, 2.4 mmol) and bis-triphenylphosphine palladium dichloride (0.56 g, 0.80 mmol). The mixture was stirred at 65° C. for 12 hours then cooled to ambient temperature and diluted with dichloromethane (20 mL) and hexane (100 mL). Celite (5 g) was added with stirring and the solids were removed by filtration. The filtrate was washed with water (600 mL). The organic layer was separated and the aqueous layer extracted with dichloromethane (3×100 mL). The combined organic solution was dried (Na₂SO₄), filtered and concentrated under reduced pressure to give a tan solid. The solid was chromatographed on silica with 3% methanol in dichloromethane to give the titled compound (4.02 g, 95%). MS (DCI) m/z 263 [M+H]⁺;

### Example 1C

### 4-[2-(2-ethyl methanesulfonyl)-1-benzofuran-5-yl]benzonitrile

To a solution of Example 1B (0.57 g, 2.2 mmol) and triethylamine (0.9 mL, 6.5 mmol) in dichloromethane (10 mL) at 20° C. was added methane sulfonyl chloride (0.79 g, 4.5 mmol). The mixture was stirred for 30 min., diluted with dichloromethane, washed with water, dried (Na₂SO₄), filtered and concentrated under reduced pressure. The residue was chromatographed on silica with dichloromethane to give the titled compound (0.66 g, 89%). MS (DCI) m/z 359 [M+H]⁺;

### Example 1D

### 4-(2-{2-[(2R)-2-methylpyrrolidinyl]ethyl}-1-benzofuran-5-yl)benzonitrile

A suspension of Example 1C (0.19 g, 0.55 mmol), 2-(R)-methyl pyrrolidine hydrobromide (0.17 g, 1.0 mmol) and sodium carbonate (0.23 g, 2.2 mmol) in acetonitrile (0.4 mL) was heated to 50° C. with stirring for 48 hours. The reaction was cooled to ambient temperature, diluted with acetonitrile and centrifuged. The supernatant liquid was removed and the solids washed with acetonitrile. The combined liquids were concentrated under reduced pressure and the residue chromatographed by reverse phase HPLC with aqueous CF₃CO₂H/acetonitrile to give the titled compound (0.065 g, 34%). ¹H NMR (300 MHz, CD₃OD) δ7.88 (m, 1H), 7.71 (m, 4H), 7.50 (m, 2H), 6.82 (s, 1H), 3.72-3.9 (m, 2H), 3.58 (m, 1H), 3.25-3.5 (m, 4H), 2.48 (m, 1H), 2.05-2.2 (m, 2H), 1.75 (m, 1H), 1.50 (d, J=6 Hz, 3H); MS (DCI) m/z 331 [M+H]⁺;

### EXAMPLE 2

### 4-{2-[2-(1-pyrrolidinyl)ethyl]-1-benzofuran-5-yl}benzonitrile

The product from Example 1C and pyrrolidine were processed as described in Example 1D to provide the titled compound. ¹H NMR (300 MHz, CD₃OD) δ7.7 (m, 5H), 7.50 (d, J=8.7 Hz, 1H), 7.42 (dd, J=8.7, 1.5 Hz, 1H), 6.51 (s, 1H), 3.1 (m, 2H), 2.95 (m, 2H), 2.65 (m, 4H), 1.9 (m, 4H); MS (DCI) m/z 317 [M+H]⁺;

### EXAMPLE 3

### 4-{2-[2-(2-methyl-1-pyrrolidinyl)ethyl]-1-benzofuran-5-yl}benzonitrile

The product from Example 1C and 2-methyl-pyrrolidine were processed as described in Example 1D to provide the titled compound. ¹H NMR (300 MHz, CD₃OD) δ7.88 (m, 1H), 7.80 (m, 4H), 7.60 (m, 2H), 6.82 (s, 1H), 3.8-3.9 (m, 2H), 3.58 (m, 1H), 3.25-3.5 (m, 4H), 2.48 (m, 1H), 2.05-2.2 (m, 2H), 1.75 (m, 1H), 1.50 (d, 3H, J=6 Hz); MS (DCI) m/z 331 [M+H]⁺;

### EXAMPLE 4

### 4-{2-[2-(1-piperidinyl)ethyl]-1-benzofuran-5-yl }benzonitrile

The product from Example 1C and piperidine were processed as described in Example 1D to provide the titled compound. ¹H NMR (300 MHz, CD₃OD) δ7.88 (m, 1H), 7.80 (m, 4H), 7.60 (m, 2H), 6.82 (s, 1H), 3.65 (m, 2H), 3.55 (m, 2H), 3.33 (m, 2H), 3.05 (m, 2H), 2.0 (m, 2H), 1.5-1.9 (m, 4H); MS (DCI) m/z 331 [M+H]⁺;

### EXAMPLE 5

### 4-{2-[2-(diethylamino)ethyl]-1-benzofuran-5-yl}benzonitrile

The product from Example 1C and diethylamine were processed as described in Example 1D to provide the titled compound. ¹H NMR (300 MHz, CD₃OD) δ7.75 (m, 1H), 7.80 (m, 4H), 7.60 (m, 2H), 6.85 (s, 1H), 3.6 (t, J=7.5 Hz, 2H), 3.25-3.5 (m, 6H), (t, 6H, J=6.6 Hz); MS (DCI) m/z 319 [M+H]⁺;

### EXAMPLE 6

### 4-{2-[2-(2-methyl-1-piperidinyl)ethyl]-1-benzofuran-5-yl }benzonitrile

The product from Example 1C and 2-methylpiperidine were processed as described in Example 1D to provide the titled compound. ¹H NMR (300 MHz, CD₃OD) δ7.88 (m, 1H), 7.80 (m, 4H), 7.60 (m, 2H), 6.82 (s, 1H), 3.1-3.8 (m, 7H), 1.6-2.1 (m, 6H), 1.45 (d, 3H, J=6 Hz); MS (DCI) m/z 345 [M+H]⁺;

### EXAMPLE 7

### 4-(2-{2-[(3R)-3-hydroxypyrrolidinyl]ethyl}-1-benzofuran-5-yl)benzonitrile

The product from Example 1C and 3-(R)-hydroxypyrrolidine were processed as described in Example 1D to provide the titled compound. ¹H NMR (300 MHz, CD₃OD) δ7.88 (m, 1H), 7.80 (m, 4H), 7.60 (m, 2H), 6.80 (s, 1H), 4.55 (bs, 1H), 3.8-3.9 (m, 4H), 3.25-3.5 (m, 4H), 2.05-2.4 (m, 2H); MS (DCI) m/z 333 [M+H]⁺;

### EXAMPLE 8

### 4-{2-[2-(1H-imidazol1--yl)ethyl]-1-benzofuran-5-yl}benzonitrile

The product from Example 1C and imidazole were processed as described in Example 1D to provide the titled compound. ¹H NMR (300 MHz, CD₃OD) δ8.88 (s, 1H), 7.88 (m, 1H), 7.80 (m, 5H), 7.60 (m, 4H), 6.75 (s, 1H), 4.7 (t, J=6.3 Hz, 2H), 3.5 (t, J=6.3 Hz, 2H); MS (DCI) m/z 314 [M+H]⁺;

### EXAMPLE 9

### 4-(2-{2-[(3S)-3-(dimethylamino)pyrrolidinyl]ethyl}-1-benzofuran-5-yl)benzonitrile

The product from Example 1C and 3-(S)-(dimethylamino)pyrrolidine were processed as described in Example 1D to provide the titled compound. ¹H NMR (300 MHz, CD₃OD) δ7.88 (m, 1H), 7.80 (m, 4H), 7.58 (m, 2H), 6.80 (s, 1H), 4.43 (m, 1H), 3.6-3.9 (m, 4H), 3.35-3.45 (m, 4H), 2.95 (s, 6H), 2.6 (m, 1H), 2.35 (m, 1H); MS (DCI) m/z 360 [M+H]⁺;

### EXAMPLE 10

### 4-(2-{2-[(2S)-2-(hydroxymethyl)pyrrolidinyl]ethyl}-1-benzofuran-5-yl)benzonitrile

The product from Example 1C and 2-(S)-(hydroxymethyl)pyrrolidine were processed as described in Example 1D to provide the titled compound. MS (DCI) m/z 347 [M+H]⁺;

### EXAMPLE 11

### 4-(2-{2-[(2R,6S)-2,6-dimethylpiperidinyl]ethyl}-1-benzofuran-5-yl)benzonitrile

The product from Example 1C and (2R,6S)-dimethylpiperidine were processed as described in Example 1D to provide the titled compound. MS (DCI) m/z 360 [M+H]⁺;

### EXAMPLE 12

### 4-(2-{2-[(2R,5R)-2,5-dimethylpyrrolidinyl]ethyl}-1-benzofuran-5-yl)benzonitrile

The product from Example 1C and (2R,5R)-dimethylpyrrolidine were processed as described in Example 1D to provide the titled compound. MS (DCI) m/z 345 [M+H]⁺;

### EXAMPLE 13

### 4-{2-[2-(1-azepanyl)ethyl]-1-benzofuran-5-yl}benzonitrile

The product from Example 1C and azepine were processed as described in Example 1D to provide the titled compound. MS (DCI) m/z 345 [M+H]⁺;

### EXAMPLE 14

### 4-{2-[2-(4-methyl-1-piperidinyl)ethyl]-1-benzofuran-5-yl}benzonitrile

The product from Example 1C and 4-methylpiperidine were processed as described in Example 1D to provide the titled compound. MS (DCI) m/z 345 [M+H]⁺;

### EXAMPLE 15

### 4-(2-{2-[2-pyrrolidine methyl carboxylate]ethyl}-1-benzofuran-5-yl)benzonitrile

The product from Example 1C and (L)-proline methyl ester were processed as described in Example 1D to provide the titled compound. MS (DCI) m/z 375 [M+H]⁺;

### EXAMPLE 16

### 4-{2-[2-(3,6-dihydro-1(2H)-pyridinyl)ethyl]-1-benzofuran-5-yl}benzonitrile

The product from Example 1C and 1,2,3,6-tetrahydropyridine were processed as described in Example 1D to provide the titled compound. MS (DCI) m/z 329 [M+H]⁺;

### EXAMPLE 17

### 4-(2-{2-[(2R)-2-(hydroxymethyl)pyrrolidinyl]ethyl}-1-benzofuran-5-yl)benzonitrile

The product from Example 1C and d-prolinol were processed as described in Example 1D to provide the titled compound. ¹H NMR (300 MHz, CD₃OD) δ7.87 (m, 1H), 7.82 (m, 4H), 7.58 (m, 2H), 6.80 (s, 1H), 3.95 (m, 2H), 3.72 (m, 2H), 3.58 (m, 1H), 3.35-3.4 (m, 4H), 1.95-2.3 (m, 4H); MS (DCI) m/z 347 [M+H]⁺;

### EXAMPLE 18

### 4-(2-{2-[tert-butyl (methyl)amino]ethyl}-1-benzofuran-5-yl)benzonitrile

The product from Example 1C and tert-butyl(methyl)amine were processed as described in Example 1D to provide the titled compound. MS (DCI) m/z 333 [M+H]⁺;

### EXAMPLE 19

### 4-(2-{2-[isopropyl(methyl)amino]ethyl}-1-benzofuran-5-yl)benzonitrile

The product from Example 1C and isopropyl(methyl)amine were processed as described in Example 1D to provide the titled compound. MS (DCI) m/z 319 [M+H]⁺;

### EXAMPLE 20

### 4-(2-{2-[isobutyl(methyl)amino]ethyl}-1-benzofuran-5-yl)benzonitrile

The product from Example 1C and isobutyl(methyl)amine were processed as described in Example 1D to provide the titled compound. MS (DCI) m/z 333 [M+H]⁺;

### EXAMPLE 21

### 4-(2-{2-[ethyl(isopropyl)amino]ethyl}-1-benzofuran-5-yl)benzonitrile

The product from Example 1C and ethyl(isopropyl)amine were processed as described in Example 1D to provide the titled compound. MS (DCI) m/z 333 [M+H]⁺;

### EXAMPLE 22

### 4-(2-{2-[ethyl(propyl)amino]ethyl}-1-benzofuran-5-yl)benzonitrile

The product from Example 1C and ethyl(propyl)amine were processed as described in Example 1D to provide the titled compound. MS (DCI) m/z 333 [M+H]⁺;

### EXAMPLE 23

### 4-(4-{2-[2-(2-methyl-1-pyrrolidinyl)ethyl]-1-benzofuran-5-yl}benzoyl)morpholine

### Example 23A

### 4′(4-morpholinylcarbonyl)[1,1′biphenyl]-4-ol

To a solution of 4-hydroxy-biphenyl-4′-carboxylic acid (5.35 g, 25.0 mmol), morpholine (2.39 g, 27.5 mmol) and triethylamine (3.5 mL, 25 mmol) in dichloromethane (100 mL) was added 1-[3-(dimethylamino)propyl]-3-ethylcarbodiimide hydrochloride. The mixture was stirred for 16 hours, diluted with aqueous NaH₂PO₄ and filtered. The solid was washed with 1:2 diethyl ether/water (100 mL) then with water (400 mL). The solid was dried in vacuo to give the titled compound (5.89 g, 83%). MS (DCI) m/z 284 [M+H]⁺;

### Example 23B

### 3-iodo-4′-(4-morpholinylcarbonyl)[1,1′-biphenyl]4-ol

The product from Example 23A was processed as described in Example 1A to provide the titled compound. MS (DCI) m/z 410 [M+H]⁺;

### Example 23C

### 2-{5-[4-(4-morpholinylcarbonyl)phenyl]-1-benzofuran-2-yl}ethanol

The product from Example 23B was processed as described in Example 1B to provide the titled compound. MS (DCI) m/z 352 [M+H]⁺;

### Example 23D

### 2-{5-[4-(4-morpholinylcarbonyl)phenyl]-1-benzofuran-2-yl}ethyl methanesulfonate

The product from Example 23C was processed as described in Example 1C to provide the titled compound.

### Example 23E

### 4-(4-{2-[2-(2-methyl1--pyrrolidinyl)ethyl]-1-benzofuran-5-yl}benzoyl)morpholine

The product from Example 23D and 2-methyl-pyrrolidine were processed as described in Example 1D to provide the titled compound. ¹H NMR (300 MHz, d₄-methanol) δ7.83 (m, 1H), 7.74 (d, J=8.4 Hz, 2H), 7.57 (m, 2H), 7.52 (d, J=8.4 Hz, 2H), 6.80 (s, 1H), 3.2-3.9 (m, 7H), 2.35 (m, 1H), 2.10 (m, 2H), 1.76 (m, 1H), 1.48 (d, J=7.2 Hz, 3H); MS (DCI) m/z 419 [M+H]⁺;

### EXAMPLE 24

### 4-(4-{2-[2-(1-piperidinyl)ethyl]-1-benzofuran-5-yl}benzoyl)morpholine

The product from Example 23D and piperidine were processed as described in Example 1D to provide the titled compound. ¹H NMR (300 MHz, CD₃OD) δ7.82 (m, 1H), 7.73 (d, J=8.1, 2H), 7.54 (m, 2H), 7.51 (d, J=8.1 Hz, 2H), 6.77(s, 1H), 3.32-3.8 (m, 14H), 3.07 (m, 2H), 1.5-2.1 (m, 6H); MS (DCI) m/z 419 [M+H]⁺;

### EXAMPLE 25

### N,N-diethyl-N-(2-{5-[4-(4-morpholinylcarbonyl)phenyl]-1-benzofuran-2-yl}ethyl)amine

The product from Example 23D and diethylamine were processed as described in Example 1D to provide the titled compound. ¹H NMR (300 MHz, CD₃OD) δ7.82 (m, 1H), 7.74 (d, J=8.1, 2H), 7.54 (m, 2H), 7.51 (d, J=8.1 Hz, 2H), 6.80(s, 1H), 3.32-3.8 (m, 16H), 1.38 (t, J=7.5 Hz, 6H); MS (DCI) m/z 407 [M+H]⁺;

### EXAMPLE 26

### 4-(4-{2-[2-(2-methyl1--piperidinyl)ethyl]-1-benzofuran-5-yl}benzoyl)morpholine

The product from Example 23D and 2-methyl-piperidine were processed as described in Example 1D to provide the titled compound. ¹H NMR (300 MHz, CD₃OD) δ7.82 (m, 1H), 7.74 (d, J=8.1, 2H), 7.56 (m, 2H), 7.52 (d, J=8.1 Hz, 2H), 6.80 (s, 1H), 3.4-3.78 (m, 15H), 1.6-2.1 (m, 6H), 1.46 (d, J=6.3 Hz, 3H); MS (DCI) m/z 433 [M+H]⁺;

### EXAMPLE 27

### (3R)-1-(2-{5-[4-(4-morpholinylcarbonyl)phenyl]-1-benzofuran-2-yl}ethyl)-3-pyrrolidinol

The product from Example 23D and 3-(R)-pyrrolidinol were processed as described in Example 1D to provide the titled compound. ¹H NMR (300 MHz, CD₃OD) δ7.82 (m, 1H), 7.73 (d, J=8.1 Hz, 2H), 7.55 (m, 2H), 7.52 (d, J=8.1 Hz, 2H), 6.78 (s, 1H), 3.35-3.8 (m, 17H), 2.3-2.4 (m, 2H); MS (DCI) m/z 421 [M+H]⁺;

### EXAMPLE 28

### 4-[4-(2-{2-[(2R,5R)-2,5-dimethylpyrrolidinyl]ethyl}-1-benzofuran-5-yl)benzoyl]morpholine

The product from Example 23D and (2R,5R)-dimethylpyrrolidine were processed as described in Example 1D to provide the titled compound. ¹H NMR (300 MHz, CD₃OD) δ7.82 (m, 1H), 7.76 (d, J=8.1, 2H), 7.56 (m, 2H), 7.52 (d, J=8.1 Hz, 2H), 6.81 (s, 1H), 3.3-3.8 (m, 14H), 1.6-2.1 (m, 4H), 1.50 (d, J=6.6 Hz, 3H), 1.38 (d, J=6.6 Hz, 3H); MS (DCI) m/z 433 [M+H]⁺;

### EXAMPLE 29

### 4-[4-(2-{2-[(2R,6S)-2,6-dimethylpiperidinyl]ethyl}-1-benzofuran-5-yl)benzoyl]morpholine

The product from Example 23D and (2R,6S)-dimethylpiperidine were processed as described in Example 1D to provide the titled compound. ¹H NMR (300 MHz, CD₃OD) δ7.82 (m, 1H), 7.74 (d, J=8.1, 2H), 7.56 (m, 2H), 7.52 (d, J=8.1 Hz, 2H), 6.80 (s, 1H), 3.45-3.85 (m, 14H), 1.6-2.1 (m, 6H), 1.48 (d, J=6.3 Hz, 6H); MS (DCI) m/z 446 [M+H]⁺;

### EXAMPLE 30

### 4-(4-{2-[2-(azepane)ethyl]-1-benzofuran-5-yl}benzoyl)morpholine

The product from Example 23D and azepane were processed as described in Example 1D to provide the titled compound. ¹H NMR (300 MHz, CD₃OD) δ7.82 (m, 1H), 7.73 (d, J=8.1, 2H), 7.56 (m, 2H), 7.52 (d, J=8.1 Hz, 2H), 6.77(s, 1H), 3.3-3.8 (m, 16H), 1.6-2.1 (m, 8H); MS (DCI) m/z 433 [M+H]⁺;

### EXAMPLE 31

### 4-(4-{2-[2-(4-methyl-1-piperidinyl)ethyl]-1-benzofuran-5-yl}benzoyl)morpholine

The product from Example 23D and 4-methyl piperidine were processed as described in Example 1D to provide the titled compound. ¹H NMR (300 MHz, CD₃OD) δ7.82 (m, 1H), 7.75 (d, J=8.1, 2H), 7.58 (m, 2H), 7.52 (d, J=8.1 Hz, 2H), 6.76 (s, 1H), 3.35-3.8 (m, 14H), 3.05 (m, 2H), 2.00 (m, 2H), 1.75 (m, 1H), 1.49 (m, 2H), 1.05 (d, J=6.6 Hz, 3H); MS (DCI) m/z 433 [M+H]⁺;

### EXAMPLE 32

### 4-(4-{2-[2-(4-morpholine)ethyl]-1-benzofuran-5-yl}benzoyl)morpholine

The product from Example 23D and morpholine were processed as described in Example 1D to provide the titled compound. ¹H NMR (300 MHz, CD₃OD) δ7.82 (m, 1H), 7.75 (d, J=8.1, 2H), 7.58 (m, 2H), 7.52 (d, J=8.1 Hz, 2H), 6.80 (s, 1H), 3.32-3.8 (m, 16H), 3.37 (t, J=7.5 Hz, 4H); MS (DCI) m/z 421 [M+H]⁺;

### EXAMPLE 33

### 4-(4-{2-[2-(3,6-dihydro-1(2H)-pyridinyl)ethyl]-1-benzofuran-5-yl}benzoyl)morpholine

The product from Example 23D and 1,2,3,6-tetrahydropyridine were processed as described in Example 1D to provide the titled compound. ¹H NMR (300 MHz, CD₃OD) δ7.83 (m, 1H), 7.74 (d, J=8.1, 2H), 7.58 (m, 2H), 7.51 (d, J=8.1 Hz, 2H), 6.80 (s, 1H), 6.05 (m, 1H), 5.79 (m, 2H), 3.4-3.8 (m, 12H), 3.41 (t, J=7.5 Hz, 4H), 2.5 (m, 2H); MS (DCI) m/z 416 [M+H]⁺;

### EXAMPLE 34

### 4-(4-{2-[2-(2S)-pyrrolidinylmethanol)ethyl]-1-benzofuran-5-yl}benzoyl)morpholine

The product from Example 23D and 2-(S)-(hydroxymethyl)pyrrolidine were processed as described in Example 1D to provide the titled compound. ¹H NMR (300 MHz, CD₃OD) δ7.81 (m, 1H), 7.73 (m, 2H), 7.55 (m, 2H), 7.50 (m 2H, J=8.4 Hz), 6.77 (s, 1H), 3.3-4.0 (m, 17H), 1.9-2.3 (m, 4H); MS (DCI) m/z 434 [M+H]⁺;

### EXAMPLE 35

### N-(tert-butyl)-N-methyl-N-(2-{5-[4-(4-morpholinylcarbonyl)phenyl]-1-benzofuran-2-yl}ethyl)amine

The product from Example 23D and tert-butyl(methyl)amine were processed as described in Example 1D to provide the titled compound. ¹H NMR (300 MHz, CD₃OD) δ7.83 (m, 1H), 7.74 (d, J=8.1, 2H), 7.55 (m, 2H), 7.51 (d, J=8.1 Hz, 2H), 6.81 (s, 1H), 3.3-3.8 (m, 12H), 2.93 (s, 3H), 1.48 (s, 9H); MS (DCI) m/z 421 [M+H]⁺;

### EXAMPLE 36

### N-isopropyl-N-methyl-N-(2-{5-[4-(4-morpholinylcarbonyl)phenyl]-1-benzofuran-2-yl}ethyl)amine

The product from Example 23D and isopropyl(methyl)amine were processed as described in Example 1D to provide the titled compound. ¹H NMR (300 MHz, CD₃OD) δ7.82 (m, 1H), 7.74 (d, J=8.1, 2H), 7.58 (m, 2H), 7.52 (d, J=8.1 Hz, 2H), 6.81 (s, 1H), 3.3-3.8 (m, 13H), 2.97 (s, 3H), 1.42 (d, 6.3 Hz, 3H), 1.37 (d, 6.3 Hz, 3H); MS (DCI) m/z 407 [M+H]⁺;

### EXAMPLE 37

### N-isobutyl-N-methyl-N-(2-{5-[4-(4-morpholinylcarbonyl)phenyl]-1-benzofuran-2-yl}ethyl)amine

The product from Example 23D and isobutyl(methyl)amine were processed as described in Example 1D to provide the titled compound. ¹H NMR (300 MHz, CD₃OD) δ7.82 (m, 1H), 7.74 (d, J=8.1, 2H), 7.58 (m, 2H), 7.51 (d, J=8.1 Hz, 2H), 6.81 (s, 1H), 3.3-3.8 (m, 14H), 2.96 (s, 3H), 2.2 (m, 1H), 1.09 (d, J=6.6 Hz, 6H); MS (DCI) m/z 421 [M+H]⁺;

### EXAMPLE 38

### N-ethyl-N-isopropyl-N-(2-{5-[4-(4-morpholinylcarbonyl)phenyl]-1-benzofuran-2-yl}ethyl)amine

The product from Example 23D and isopropyl(ethyl)amine were processed as described in Example 1D to provide the titled compound. ¹H NMR (300 MHz, CD₃OD) δ7.83 (m, 1H), 7.74 (d, J=8.1, 2H), 7.58 (m, 2H), 7.53 (d, J=8.1 Hz, 2H), 6.80 (s, 1H), 3.3-3.8 (m, 15H), 1.41 (m, 9H); MS (DCI) m/z 421 [M+H]⁺;

### EXAMPLE 39

### N,N-dimethyl-N-(2-{5-[4-(4-morpholinylcarbonyl)phenyl]-1-benzofuran-2-yl}ethyl)amine

The product from Example 23D and dimethylamine were processed as described in Example 1D to provide the titled compound. MS (DCI) m/z 378 [M+H]⁺;

### EXAMPLE 40

### N-ethyl-N-(2-{5-[4-(4-morpholinylcarbonyl)phenyl]-1-benzofuran-2-yl}ethyl)-N-propylamine

The product from Example 23D and ethyl(propyl)amine were processed as described in Example 1D to provide the titled compound. ¹H NMR (300 MHz, CD₃OD) δ7.84 (m, 1H), 7.74 (d, J=8.1, 2H), 7.58 (m, 2H), 7.53 (d, J=8.1 Hz, 2H), 6.82(s, 1H), 3.32-3.8 (m, 14H), 3.20 (m, 2H), 1.80 (m, 2H), 1.38 (t, J=7.5 Hz, 3H), 1.05 (t, J=7.5 Hz, 3H); MS (DCI) m/z 421 [M+H]⁺;

### EXAMPLE 41

### 4-{4-methyl-2-oxo-3-[2-(1-pyrolidinyl)ethyl]-2H-chromen-6-yl}benzonitrile

### Example 41A

### 3-(2-bromoethyl)-6-hydroxy-4-methyl-2H-chromen-2-one

To a solution of resorcinol (7.03 g, 64.0 mmol) in a solution consisting of HBr (104 mL, 422 mmol) and glacial acetic acid (10 mL) at 0° C. was slowly added 2-acetylbutyrolactone (5.8 mL, 54 mmol). The mixture was warmed to ambient temperature and then heated to reflux for 2 hours. The mixture was cooled to ambient temperature and diluted with water (350 mL). The mixture was filtered and the solid dried in vacuo overnight to give the titled compound (15.5 g, 85%). ¹H NMR (300 MHz, CD₃OD) δ10.5 (s, 1H), 7.6 (d, J=8.7 Hz, 1H), 6.8 (dd, J=6.6 Hz, 11.4 Hz, 1H), 6.7 (d, J=2.1 Hz, 1H), 3.6 (t, J=7.5 Hz, 2H), 3.1 (t, J=7.6 Hz, 2H), 2.4 (s, 3H); MS (DCI) m/z 283, 284 [M+H]⁺;

### Example 41B

### 6-hydroxy-4-methyl-3-[2-(1-pyrrolidinyl)ethyl]-2H-chromen -2-one

A solution of Example 41A (0.20 g, 0.70 mmol) and pyrrolidine (0.50 mL, 6.0 mmol) in DMF (2 mL) was heated to 75° C. for 16 hours, cooled to ambient temperature, diluted with water (20 mL) and extracted with ethyl acetate (3×50 mL). The combined ethyl acetate was dried (MgSO₄), filtered, concentrated under reduced pressure and chromatographed on silica with 10% methanol in dichloromethane to give the titled compound (0.48 g, 25%). MS (DCI) m/z 274 [M+H]⁺;

### Example 41C

To a solution of Example 41B (0.105 g, 0.38 mmol), N-phenyltrifluoromethane sulfonimide (0.143 g, 0.38 mmol) in dichloromethane (2 mL) at 0° C. was added triethylamine (0.68 mL, 0.48 mmol). The mixture was stirred at ambient temperature for 12 hours, diluted with diethyl ether (40 mL) and washed sequentially with aqueous NaOH (1N, 2×30mL), water and brine, dried (MgSO₄), filtered and evaporated to provide the triflate. A mixture of the triflate (0.2 g, 0.49 mmol), 4-cyanophenylboronic acid (0.082 g, 0.54 mmol), Pd(PPh₃)₂Cl₂ (0.035 g) and Cs₂CO₃ (0.96 g, 2.9 mmol) in DMF (5 mL) was stirred for 5 hours, diluted with ethyl acetate and washed sequentially with aqueous NaOH (1N, 3×25 mL), water (3×25 mL) and brine. The organic solution was dried (MgSO₄), filtered, evaporated under reduced pressure. The residue was chromatographed on silica with dichloromethane ethyl acetate methanol to give the titled compound. NMR (300 MHz, CDCl₃) δ7.75 (m, 3H) 7.5 (m, 1H), 7.2 (m 2H), 7.1 (m, 1H) 3.1 (m, 2H), 2.9 (m, 4H), 2.5 (s, 3H), 2.0, (m, 4H), 1.6 (m, 2H); MS (DCI) m/z 359 [M+H]⁺;

### EXAMPLE 42

### 4-{4-methyl-2-oxo-3-[2-(1-piperidinyl)ethyl]-2H-chromen-6-yl}benzonitrile

The product from Example 41A and piperidine were processed as described in Examples 41B and 41C to provide the titled compound. NMR (300 MHz, CDCl₃) δ7.75 (m, 3H) 7.6 (m, 3H), 7.2 (m, 1H), 2.95 (m, 2H), 2.6 (m, 6H), 2.5 (s, 3H), 1.7 (m, 4H), 1.5 (m, 2H); MS (DCI) m/z 373 [M+H]⁺;

### EXAMPLE 43

### 4-{3-[2-(diethylamino)ethyl]-4-methyl-2-oxo-2H-chromen-6-yl}benzonitrile

The product from Example 41A and diethylamine were processed as described in Examples 41B and 41C to provide the titled compound. NMR (300 MHz, CDCl₃) δ7.75 (m, 5H), 7.5 (m, 2H), 2.9 (m, 2H), 2.7 (m, 6H), 2.5 (s, 3H), 1.1 (t, J=9 Hz, 6H); MS (DCI) m/z 361 [M+H]⁺;

### EXAMPLE 44

### 4-[(6-{2-[2-(1-pyrrolidinyl)ethyl]-1-benzofuran-5-yl}-3-pyridinyl)carbonyl]morpholine

### Example 44A

### 4-[(6-chloro-3-pyridinyl)carbonyl]morpholine

To a solution of chloronicotinoyl chloride (3.52 g, 2.00 mmol) and triethylamine (3.1 mL, 2.22 mmol) in dichloromethane (5 mL) at 0° C. was slowly added morpholine (1.75 mL, 2.00 mmol). The mixture was warmed to ambient temperature, washed with water (2×25 mL), brine (1×25 mL), dried (Na₂SO₄), filtered and concentrated under reduced pressure. The product was chromatographed on silica with ethyl acetate to give the titled compound (4.0 g, 88%). MS (DCI) m/z 227 [M+H]⁺;

### Example 44B

### 4-[5-(4-morpholinylcarbonyl)-1,6-dihydro-2-pyridinyl]phenol

A mixture of Example 44A (4.0 g, 17.6 mmol), Pd(Ph₃P)₄ (1.0 g, 0.86 mmol), 4-O-tert-butyldimethylsilyl-phenylboric acid (4.9 g, 23.6 mmol) in toluene (60 mL) and aqueous sodium carbonate (2.76 g dissolved in 25 mL water) was heated to reflux for 12 hours, then cooled to ambient temperature. The mixture was diluted with ethyl acetate (100 mL), washed with water (1×50 mL), dried (Na₂SO₄), filtered and concentrated under reduced pressure. The residue was stirred in THF (200 mL) containing tetrabutylammonium fluoride (30 mL, 1.0M, 30.0 mmol) for 16 hours. The mixture was diluted with ethyl acetate (100 mL), washed sequentially with water (1×50 mL), aqueous ammonium chloride (1×50 mL), brine (1×50 mL), dried (Na₂SO₄), filtered and concentrated under reduced pressure to give a solid. The solid was precipitated from ethyl acetate (75 mL), filtered to provide the titled compound as a tan solid (4.27 g, 70%).

### Example 44C

### 2-iodo-4-[5-(4-morpholinylcarbonyl)-2-pyridinyl]phenol

A mixture of Example 44B (4.25 g, 15.0 mmol) sodium iodide (2.36 g, 15.7 mmol) and sodium hydroxide (0.63 g, 15.7 mmol) was stirred in methanol (90 mL) with heating until a clear solution was obtained. The solution was then cooled to 0° C. and to this was slowly added sodium hypochlorite (22 mL of 5.25%, 1.15 g, 15.5 mmol) (Clorox™) over 45 minutes. While maintaining 0° C., two sequential additions of NaI (0.3 g, 1.5 mmol) and Clorox (2.2 mL, 0.12 g, 1.5 mmol) were made both 2 hours and 4 hours later. The mixture was stirred for 12 hours at ambient temperature, quenched by the sequential addition of aqueous sodium thiosulfate (10 mL), water (800 mL) and sufficient aqueous sodium dihydrogen phosphate (NaH₂PO₄) to adjust the pH to 7. The mixture was extracted with dichloromethane, and the combined extracts dried (Na₂SO₄), filtered and concentrated under reduced pressure to give a tan foam. The product was crystallized from ethanol to give the titled compounds as a tan solid (3.78 g, 61%). MS (DCI) m/z 411 [M+H]⁺;

### Example 44D

### 2-{5-[5-(4-morpholinylcarbonyl)-2-pyridinyl]-1-benzofuran-2-yl}ethanol

To a solution of Example 44C (2.85 g, 6.95 mmol), triethylamine (2.4 mL, 17.4 mmol), and 3-butyn-1-ol (0.73 g, 10.4 mmol) in dimethylformamide (15 mL) at 20° C. was added cuprous iodide (0.2 g, 1.0 mmol) and bis-triphenylphosphine palladium dichloride (0.24 g, 0.35 mmol). The mixture was stirred for one hour, then heated to 65° C. for 16 hours. The reaction was cooled to 23° C., diluted with dichloromethane (200 mL) and water (100 mL). The mixture was stirred with Celite then filtered. The filtrate was washed with water (1×50 mL), the organic phase separated, dried (Na₂SO₄), filtered and concentrated under reduced pressure to give a tan foam. The residue was chromatographed on silica using 5% methanol in dichloromethane to give the titled compound as a yellow solid (1.83 g, 75%). MS (DCI) m/z 353 [M+H]⁺;

### Example 44E

### 2-{5-[5-(4-morpholinylcarbonyl)-2-pyridinyl]-1-benzofuran-2-yl}ethyl methanesulfonate

The product from Example 44D was processed as described in Example 1C to provide the titled compound.

### Example 44F

### 4-[(6-{2-[2-(1-pyrrolidinyl)ethyl]-1-benzofuran-5-yl}-3-pyridinyl)carbonyl]morpholine

The product from Example 44E and pyrrolidine were processed as described in Example 1D to provide the titled compound. ¹H NMR (300 MHz, CD₃OD) δ8.70 (m, 1H), 8.24 (d, J=1.8 Hz, 1H), 7.98 (m, 3H), 7.60 (d, J=8.7 Hz, 1H), 6.84 (s, 1H), 3.3-3.8 (m, 12H), 3.18 (m, 2H), 2.0-2.25 (m, 6H); MS (DCI) m/z 406 [M+H]⁺;

### EXAMPLE 45

### 4-{[6-(2-{2-[(2R)-methylpyrrolidinyl]ethyl}-1-benzofuran-5-yl)-3-pyridinyl]carbonyl}morpholine

The product from Example 44E and 2-(R)-methylpyrrolidine were processed as described in Example 1D to provide the titled compound. MS (DCI) m/z 420 [M+H]⁺;

### EXAMPLE 46

### 4-[(6-{2-[2-(1-piperidinyl)ethyl]-1-benzofuran-5-yl}-3-pyridinyl)carbonyl]morpholine

The product from Example 44E and piperidine were processed as described in Example 1D to provide the titled compound. ¹H NMR (300 MHz, CD₃OD) δ8.70 (m, 1H), 8.22 (d, J=1.8 Hz, 1H), 7.95 (m, 3H), 7.58 (d, J=8.7 Hz, 1H), 6.82 (s, 1H), 3.3-3.8 (m, 12H), 3.05 (m, 2H), 1.5-2.0 (m, 8H); MS (DCI) m/z 420 [M+H]⁺;

### EXAMPLE 47

### 4-[(6-{2-[2-(N,N-diethyl)ethyl]-1-benzofuran-5-yl}-3-pyridinyl)carbonyl]morpholine

The product from Example 44E and diethylamine were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.84 (s, 1H), 3.3-3.8 (m, 12H), 1.38 (t, J=7.5 Hz, 6H); MS (DCI) m/z 408 [M+H]⁺;

### EXAMPLE 48

### (3R)-1-(2-{5-[5-(4-morpholinylcarbonyl)-2-pyridinyl]-1-benzofuran-2-yl}ethyl)-3-pyrrolidinol

The product from Example 44E and 3-(R)-hydroxypyrrolidine were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.82 (s, 1H), 4.55 (m, 1H), 3.3-3.8 (m, 16H), 2.0-2.4 (m, 2H); MS (DCI) m/z 422 [M+H]⁺;

### EXAMPLE 49

### 4-{[6-(2-{2-[(2R,5R)-2,5-dimethylpyrrolidinyl]ethyl}-1-benzofuran-5-yl)-3-pyridinyl]carbonyl}morpholine

The product from Example 44E and (2R,5R)-dimethylpyrrolidine were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.86 (s, 1H), 3.3-3.8 (m, 12H), 1.2-2.4 (m, 10H); MS (DCI) m/z 434 [M+H]⁺;

### EXAMPLE 50

### 4-{[6-(2-{2-[(2R,6S)-2,6-dimethylpiperidinyl]ethyl}-1-benzofuran-5-yl)-3-pyridinyl]carbonyl}morpholine

The product from Example 44E and (2R,6S)-dimethylpiperidine were processed as described in Example 1D to provide the titled compound. ¹H NMR (300 M, CD₃OD) δ8.70 (m, 1H), 8.24 (d, J=1.8 Hz, 1H), 7.95 (m, 3H), 7.58 (d, J=8.7 Hz, 1H), 6.86 (s, 1H), 3.45-3.85 (m, 12H), 1.6-2.1 (m, 6H), 1.48 (d, J=6.3 Hz, 6H); MS (DCI) m/z 448 [M+H]⁺;

### EXAMPLE 51

### 4-{[6-(2-{2-[1-azepanyl]ethyl}-1-benzofuran-5-yl)-3-pyridinyl]carbonyl}morpholine

The product from Example 44E and azepine were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.86 (s, 1H), 3.3-3.8 (m, 16H), 1.95 (m, 4H), 1.75 (m, 4H); MS (DCI) m/z 434 [M+H]⁺;

### EXAMPLE 52

### 4-[(6-{2-[2-(4-methyl-1-piperidinyl)ethyl]-1-benzofuran-5-yl}-3-pyridinyl)carbonyl]morpholine

The product from Example 44E and 4-methylpiperidine were processed as described in Example 1D to provide the titled compound. ¹H NMR (300 MHz, CD₃OD) δ8.70 (m, 1H), 8.22 (d, J=1.8 Hz, 1H), 7.97 (m, 3H), 7.58 (d, J=8.7 Hz, 1H), 6.82 (s, 1H), 3.3-3.8 (m, 14H), 3.05 (m, 2H), 1.95 (m, 2H), 1.75 (m, 1H), 1.5 (m, 2H), 1.05 (d, J=6.6 Hz, 3H); MS (DCI) m/z 434 [M+H]⁺;

### EXAMPLE 53

### 4-[(6-{2-[2-(4-morpholinyl)ethyl]-1-benzofuran-5-yl}-3-pyridinyl)carbonyl]morpholine

The product from Example 44E and morpholine were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.82 (s, 1H), 3.3-4.1 (m, 16H), 3.37 (t, J=7.5 Hz, 4H); MS (DCI) m/z 422 [M+H]⁺;

### EXAMPLE 54

### N-(tert-butyl)-N-methyl-N-(2-{5-[5-(4-morpholinylcarbonyl)-2-pyridinyl]-1-benzofuran-2-yl}ethyl)amine

The product from Example 44E and tert-butyl(methyl)amine were processed as described in Example 1D to provide the titled compound. ¹H NMR (300 MHz, CD₃OD) δ8.70 (m, 1H), 8.24 (d, J=1.8 Hz, 1H), 7.96 (m, 3H), 7.59 (d, J=8.7 Hz, 1H), 6.85 (s, 1H), 3.3-3.8 (m, 12H), 2.93 (s, 3H), 1.48 (s, 9H); MS (DCI) m/z 422 [M+H]⁺;

### EXAMPLE 55

### N-isobutyl-N-methyl-N-(2-{5-[5-(4-morpholinylcarbonyl)-2-pyridinyl]-1-benzofuran-2yl}ethyl)amine

The product from Example 44E and isobutyl(methyl)amine were processed as described in Example 1D to provide the titled compound. ¹H NMR (300 MHz, CD₃OD) δ8.70 (m, 1H), 8.23 (d, J=1.8 Hz, 1H), 7.98 (m, 3H), 7.59 (d, J=8.7 Hz, 1H), 6.85 (s, 1H), 3.0-3.8 (m, 14H), 2.98 (s, 3H), 2.2 (m, 1H), 1.09 (d, J=6.6 Hz, 6H); MS (DCI) m/z 422 [M+H]⁺;

### EXAMPLE 56

### N-isopropyl-N-methyl-N-(2-{5-[5-(4-morpholinylcarbonyl)-2-pyridinyl]-1-benzofuran-2-yl}ethyl)amine

The product from Example 44E and isopropyl(methyl)amine were processed as described in Example 1D to provide the titled compound. ¹H NMR (300 MHz, CD₃OD) δ8.70 (m, 1H), 8.24 (d, J=1.8 Hz, 1H), 7.96 (m, 3H), 7.59 (d, J=8.7 Hz, 1H), 6,85 (s, 1H), 3.3-3.8 (m, 13H), 2.88 (s, 3H), 1.40 (d, 6.3 Hz, 3H), 1.36 (d, 6.3 Hz, 3H); MS (DCI) m/z [M+H]⁺;

### EXAMPLE 57

### N-ethyl-N-isopropyl-N-(2-{5-[5-(4-morpholinylcarbonyl)-2-pyridinyl]-1-benzofuran-2-yl}ethyl)amine

The product from Example 44E and ethyl(isopropyl)amine were processed as described in Example 1D to provide the titled compound. ¹H NMR (300 MHz, CD₃OD) δ8.70 (m, 1H), 8.25 (d, J=1.8 Hz, 1H), 7.98 (m, 3H), 7.59 (d, J=8.7 Hz, 1H), 6.85 (s, 1H), 3.3-3.8 (m, 15H), 1.4 (m, 9H); MS (DCI) m/z 422 [M+H]⁺;

### EXAMPLE 58

### N,N-dimethyl-N-(2-{5-[5-(4-morpholinylcarbonyl)-2-pyridinyl]-1-benzofuran-2-yl}ethyl)amine

The product from Example 44E and dimethylamine were processed as described in Example 1D to provide the titled compound. ¹H NMR (300 MHz, CD₃OD) δ8.70 (m, 1H), 8.24 (d, J=1.8 Hz, 1H), 7.96 (m, 3H), 7.59 (d, J=8.7 Hz, 1H), 6.84 (s, 1H), 3.35-3.8 (m, 12H), 2.98 (s, 6H); MS (DCI) m/z 380 [M+H]⁺;

### EXAMPLE 59

### N-ethyl-N-propyl-N-(2-{5-[5-(4-morpholinylcarbonyl)-2-pyridinyl]-1-benzofuran-2-yl}ethyl)amine

The product from Example 44E and ethyl(propyl)amine were processed as described in Example 1D to provide the titled compound. ¹H NMR (300 MHz, CD₃OD) δ8.70 (m, 1H), 8.23 (d, J=1.8 Hz, 1H), 7.98 (m, 3H), 7.58 (d, J=8.7 Hz, 1H), 6.85 (s, 1H), 3.2-3.8 (m, 14H), 3.20 (m, 2H), 1.80 (m, 2H), 1.38 (t, J=7.5 Hz, 3H), 1.05 (t, J=7.5 Hz, 3H); MS (DCI) m/z 422 [M+H]⁺;

### EXAMPLE 60

### 8-(2-{5-[5-(4-morpholinylcarbonyl)-2-pyridinyl]-1-benzofuran-2-yl}ethyl)-1,4-dioxa-8-azaspiro[4.5]decane

The product from Example 44E and 1,4-dioxa-8-azaspiro[4.5]decane were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.82 (s, 1H), 3.3-4.1 (m, 20H), 2.05 (m, 4H); MS (DCI) m/z 478 [M+H]⁺;

### EXAMPLE 61

### 5-(2-{5-[5-(4-morpholinylcarbonyl)-2-pyridinyl]-1-benzofuran-2-yl}ethyl)-2-oxa-5-azabicyclo[2.2.1]heptane

The product from Example 44E and 2-oxo-5-azabicyclo[2.2.1]heptane were processed as described in Example 1D to provide the titled compound. ¹H NMR (300 MHz, CD₃OD) δ8.70 (m, 1H), 8.24 (d, J=1.8 Hz, 1H), 7.95 (m, 3H), 7.58 (d, J=8.7 Hz, 1H), 6.82 (s, 1H), 4.7 (m, 1H), 4.55 (m, 1H), 3.3-4.0 (m, 14H), 2.4 (m, 2H), 2.2 (m, 2H); MS (DCI) m/z 434 [M+H]⁺;

### EXAMPLE 62

### (2S)-1-(2-{5-[5-(4-morpholinylcarbonyl)-2-pyridinyl]-1-benzofuran-2-yl}ethyl)-2-pyrrolidinol

The product from Example 44E and 2-(R)-hydroxymethylpyrrolidine were processed as described in Example 1D to provide the titled compound. ¹H NMR (300 MHz, CD₃OD) δ8.70 (m, 1H), 8.23 (d, J=1.8 Hz, 1H), 7.98 (m, 3H), 7.57 (d, J=8.7 Hz, 1H), 6.82 (s, 1H), 3.3-4.0 (m, 15H), 1.9-2.3 (m, 6H); MS (DCI) m/z 436 [M+H]⁺;

### EXAMPLE 63

### N-allyl-N-(2-{5-[5-(4-morpholinylcarbonyl)-2-pyridinyl]-1-benzofuran-2-yl}ethyl)amine

The product from Example 44E and allylamine were processed as described in Example 1D to provide the titled compound. ¹H NMR (300 MHz, CD₃OD) δ8.70 (m, 1H), 8.24 (d, J=1.8 Hz, 1H), 7.96 (m, 3H), 7.59 (d, J=8.7 Hz, 1H), 6.81 (s, 1H), 5.95 (m, 1H), 5.55 (m, 2H), 3.25-3.8 (m, 14H); MS (DCI) m/z 392 [M+H]⁺;

### EXAMPLE 64

### 3-[(2-{5-[5-(4-morpholinylcarbonyl)-2-pyridinyl]-1-benzofuran-2-yl}ethyl)amino]-1-propanol

The product from Example 44E and 3-amino-1-propanol were processed as described in Example 1D to provide the titled compound. ¹H NMR (300 MHz, CD₃OD) δ8.70 (m, 1H), 8.23 (d, J=1.8 Hz, 1H), 7.98 (m, 3H), 7.59 (d, J=8.7 Hz, 1H), 6.82 (s, 1H), 3.20-3.8 (m, 16H), 1.92 (m, 2H); MS (DCI) m/z 410 [M+H]⁺;

### EXAMPLE 65

### N-(2-{5-[5-(4-morpholinylcarbonyl)-2-pyridinyl]-1-benzofuran-2-yl}ethyl)-N-propylamine

The product from Example 44E and propylamine were processed as described in Example 1D to provide the titled compound. ¹H NMR (300 MHz, CD₃OD) δ8.70 (m, 1H), 8.24 (d, J=1.8 Hz, 1H), 7.98 (m, 3H), 7.58 (d, J=8.7 Hz, 1H), 6.82 (s, 1H), 3.25-3.8 (m, 12H), 3.05 (t, J=7.5 Hz, 2H), 1.74 (m, 2H), 1.05 (t, J=7.5 Hz, 3H); MS (DCI) m/z 394 [M+H]⁺;

### EXAMPLE 66

### 4-(2-{2-[(3R)-3-(dimethylamino)pyrrolidinyl]ethyl}-1-benzofuran-5-yl)benzonitrile

The product from Example 1C and 3-(R)-(dimethylamino)pyrrolidine were processed as described in Example 1D to provide the titled compound. MS (DCI) m/z 360 [M+H]⁺;

### EXAMPLE 67

### 4-(2-{2-[(2R)-2-methylpyrrolidinyl]ethyl}-2,3-dihydro-1-benzofuran-5-yl)benzonitrile

### EXAMPLE 67A

### 4-{2-[(E)-2-methoxyethenyl]-2,3-dihydro-1-benzofuran-5-yl}benzonitrile

A solution of Example 1A (0.20 g, 0.623 mmol), 1-methoxybutadiene (0.18 g, 2.18 mmol), palladium diacetate (0.007 g, 0.031 mmol), sodium bicarbonate (0.261 g, 3.11 mmol) and tetrabutyl ammonium chloride (0.173 g, 0.623 mmol) was heated at 60° C. in DMF (3 mL) under an atmosphere of nitrogen for 36 hours. The reaction was cooled to 23° C., diluted with CH₂Cl₂ (50 mL), filtered through Celite. The solution was concentrated under reduce pressure and the residue was purified on silica using CH₂Cl₂ to give the titled compound (0.95 g, 55%). ¹H NMR (CDCl₃): 3.05 (m, 1H), 3.40 (m, 1H), 3.60 (s, 3H), 5.00 (m, 1H), 5.22 (m, 1H), 6.72 (d, J=14 Hz, 1H), 6.83 (d, J=7 Hz, 1H), 7.38 (m, 2H), 7.65 (m, 4H); MS (DCI): 278 (M+H⁺), 295 (M+NH₄⁺).

### EXAMPLE 67B

### 4-[2-(2-oxoethyl)-2,3-dihydro-1-benzofuran-5-yl]benzonitrile

A solution of Example 67A (0.5 g, 1.8 mmol) in acetone (10 mL) and p-toluenesulfonic acid monohydrate (0.51 g, 2.7 mmol) was stirred for 45 minutes, diluted with CH₂Cl₂ (150 mL), washed with cold aqueous sodium bicarbonate (2×100 mL, 10% solution), water (2×100 mL), dried (Na₂SO₄), filtered and concentrated under reduced pressure. The residue was purified on silica using CH₂Cl₂ to give the titled compound (0.41 g, 87%). ¹H NMR (CDCl₃): 2.95 (m, 2H), 3.65 (m, 3H), 6.82 (d, J=7 Hz, 1H), 7.40 (m, 2H), 7.62 (m, 4H), 9.55(d, J=7 Hz, 1H); MS (DCI) 263 (M⁺), 281 (M+NH₄⁺).

### EXAMPLE 67C

### 4-[2-(2-hydroxyethyl)-2,3-dihydro-1-benzofuran-5-yl]benzonitrile

A solution of Example 67B (0.25 g, 0.95 mmol) and sodium borohydride (0.054 g, 1.42 mmol)in methanol (5 mL) was stirred for 1 hour, cooled on ice and quenched with aqueous NaHCO₃ (50 mL). The mixture was extracted with dichloromethane (3×100 mL), the organic extracts combined and washed with water (1×150 mL), brine (1×150 mL), dried (Na₂SO₄), filtered and concentrated under reduced pressure to provide the titled compound (0.24 g, 95%). ¹H NMR (CDCl₃): 1.90 (m, 2H), 2.90 (m, 2H), 3.46 (m, 2H), 5.40 (m, 1H), 6.85 (d, J=7 Hz, 1H), 7.38 (m, 2H), 7.65 (m, 4H); MS (DCI) 265 (M⁺), 283 (M+NH₄⁺).

### EXAMPLE 67D

### 2-[5-(4-cyanophenyl)-2,3-dihydro-1-benzofuran-2-yl]ethyl methanesulfonate

To a solution of Example 67C (0.23 g, 0.87 mmol) and methanesulfonyl chloride (0.075 mL, 0.96 mmol) in CH₂Cl₂ (5 mL) at 0° C. was added triethylamine (0.13 mL, 9.4 mmol). The mixture was stirred for 30 minutes, diluted with CH₂Cl₂ (75 mL), washed with water (3×100 mL), dried (Na₂SO₄), filtered and concentrated under reduced pressure. The residue was purified on silica using CH₂Cl₂ to provide the titled compound (0.278 g, 90%). ¹H NMR (CDCl₃): H NMR: 1.95 (m, 2H), 2.93 (m, 2H), 3.00 (s, 3H), 3.46 (m, 2H), 5.00 (m, 1H), 6.81 (d, J=7 Hz, 1H), 7.40 (m, 2H), 7.65 (m, 4H); MS (DCI) 343 (M⁺), 361 (M+NH₄⁺).

### EXAMPLE 67E

### 4-(2-{2-[(2R)-2-methylpyrrolidinyl]ethyl}2,3-dihydro-1-benzofuran-5-yl)benzonitrile

A solution of Example 67D (0.2 g, 0.58 mmol), R-2-methylpyrrolidine-(L)-tartrate 1.45 mmol) and cesium carbonate (0.95 g) in acetonitrile (5 mL) was heated to 60° C. for 48 hours under an atmophere of nitrogen. The reaction was allowed to come to ambient temperature, diluted with CH₂Cl₂ (100 mL), washed with aqueous NaHCO₃ (2×100 mL), H₂O (1×100 mL), brine (100 mL), dried (Na₂SO₄), filtered and concentrated under reduced pressure. The residue was purified by on silica using CHCl₃/CH₃OH/NH₄OH (95:5:0.5) to provide the titled compound (0.14 g, 72%). ¹H NMR (CDCl₃): 1.10 (d, J=7 Hz, 2H), 1.50 (m, 2H), 1.70 (m, 4H), 1.98 (m, 2H), 2.10 (m, 2H), 2.25 (m, 1H), 3.00 (m, 2H), 4.60 (m, 1H), 6.80 (d, J=7 Hz, 1H), 7.45 (m, 2H), 7.70 (m, 4H); MS(ESI) 333 (M+H⁺).

### EXAMPLE 68

### (4-fluorophenyl)(2-{2-[(2R)-2-methyl-1-pyrrolidinyl]ethyl}-1-benzofuran-5-yl)methanone

### EXAMPLE 68A

### (4-fluorophenyl)(4-hydroxy-3-iodophenyl)methanone

(4-Fluorophenyl)(4-hydroxyphenyl)methanone (20.0 g, 92.5 mmol) in concentrated ammonium hydroxide (770 mL) was allowed to stir at 25° C. for 15 minutes and then treated with potassium iodide (74.79 g, 450.5 mmol) and iodide chips (23.48 g, 92.5 mmol) in water (185 mL). The reaction mixture was allowed to stir at 25° C. for 18 hours and then filtered. The precipitate was dissolved in ethyl acetate, washed with water and brine, dried, filtered and the filtrate concentrated under reduced pressure to provide the title compound as a pale green solid (23.4 g, 74% yield). ¹HNMR (300 MHZ, CD₃OD) δ6.91 (d, 1H, J=8.9 Hz), 7.26 (t, 2H), 7.64 (d, 1H, J=8.9 Hz), 7.78 (t, 2H), 8.17 (s, 1H); MS (DCI) m/z 342.9 (M+H)⁺, 360 (M+NH₄)⁺.

### EXAMPLE 68B

### (2R)-1-(3-butynyl)-2-methylpyrrolidine

(R)-2-methylpyrrolidine (L) tartrate (1.65 g, 7.00 mmol) and 325 mesh powdered K₂CO₃ (2.03 g, 14.7 mmol) in acetonitrile (60 mL) were heated at 50° C. in a sealed bottle for 24 hours. The mixture was allowed to cool to room temperature and was treated with 3-butynyl 4-methylbenzenesulfonate (1.24 mL, 7.0 mmol). The mixture was stirred for one hour at room temperature and then was heated at 50° C. for 24 hours. The mixture was allowed to cool to room temperature, filtered, and the filter cake washed with acetonitrile. The filtrate was diluted to a total volume of 70 mL with acetonitrile and used as a 0.1M solution in subsequent steps.

### EXAMPLE 68C

### (4-fluorophenyl)(2-{2-[(2R)-2-methyl-1-pyrrolidinyl]ethyl}1-benzofuran-5-yl)methanone

The product from Example 68A (6.5 g, 18.5 mmol) was sequentially treated with a 0.1M solution of the product from Example 68B in acetonitrile (230 mL, 23.0 mmol), Pd(OAc)₂ (0.127 g, 0.566 mmol), tris(4-methylphenyl)phosphine (0.344 g, 1.130 mmol), and copper iodide (1.08 g, 95.72 mmol). After stirring at 25° C. for 10 minutes, the reaction mixture was treated with diisopropyl amine (26.6 mL, 189 mmol) and then heated at 60° C. in an inert atmosphere for 16 hours. The reaction mixture was allowed to cool to room temperature, filtered through celite, and the filtrate concentrated under reduced pressure. The residue was purified on silica gel using 90% DCM, 9.9% MeOH, 0.1% NH₄OH to provide the title compound (1.21 g, 18.0% yield). ¹HNMR (300 MHz, CD₃OD) δ1.09 (d, 3H, J=6.1 Hz), 1.46 (m, 1H), 1.81 (m, 2H), 2.02-2.28 (m, 2H), 2.49 (m, 2H) 3.06 (m, 2H), 3.28 (m, 2H), 6.68 (s, 1H),), 7.27 (t, 2H), 7.58 (d, 1H, J=8.9 Hz), 7.71 (d, 1H, J=8.9 Hz) 7.86 (t, 2H), 7.97 (s, 1H); MS (ESI) m/z 352 (M+H)⁺.

### EXAMPLE 69

### (3-fluorophenyl)(2-{2-[(2R)-2-methyl-1-pyrrolidinyl]ethyl}-1-benzofuran-5-yl)methanone

### EXAMPLE 69A

### (3-fluorophenyl)(4-hydroxyphenyl)methanone

(3-fluorophenyl)(4-methoxyphenyl)methanone (1.0 g, 4.34 mmol) in 50 mL DCM at −78° C. while stirring was treated with 1M boron tribromide (13.03 mL, 13.03 mmol) dropwise over 20 minutes. The mixture was allowed to warm to 25° C. and stir for 18 hours. The mixture was treated with water (1 mL) and stirred for 5 minutes, followed by additional water (2 mL) and stirred for 10 minutes, and finally treated with more water (50 mL) and stirred for 20 minutes. The mixture was then extracted with DCM (50 mL×2). The organic layers were combined, dried, filtered, and the filtrate evaporated under reduced pressure. The residue was purified by flash chromatography to provide the title compound (0.69 g, 74% yield). ¹HNMR (300 MHz, CD₃OD) δ6.92 (d, 2H, J=8.9 Hz), 7.26 (m, 1H), 7.41-7.58 (m, 3H), 7.79 (d, 2H, J=8.9 Hz); MS (DCI) m/z 217 (M+H)⁺, 234 (M+NH₄)⁺.

### EXAMPLE 69B

### (3-fluorophenyl)(4-hydroxy-3-iodophenyl)methanone

The product from Example 69A, potassium iodide and iodide chips were processed as described in Example 68A to provide the title compound. ¹HNMR (300 MHz, CD₃OD) δ6.92 (d, 1H, J=8.9 Hz), 7.31-7.59 (m, 4H), 7.67 (d, 1H, J=8.9 Hz), 8.18 (s, 1H); MS (DCI) m/z 343 (M+H)⁺, 360 (M+NH₄)⁺.

### EXAMPLE 69C

### (3-fluorophenyl)(2-{2-[(2R)-2-methyl-1-pyrrolidinyl]ethyl}-1-benzofuran-5-yl)methanone

The product from Example 69B and a 0.1M solution of the product from Example 68B were processed as described in Example 68C to provide the title compound. ¹HNMR (300 MHz, CD₃OD) δ1.26 (d, 3H, J=6.1 Hz), 1.57 (m, 1H), 1.91 (m, 2H), 2.10-2.63 (m 2H), 2.87 (m, 2H) 3.18 (m, 2H), 3.42 (m, 2H), 6.76 (s, 1H), 7.37-7.59 (m, 5H), 7.76 (d, 1H, J=8.9 Hz), 7.98 (s, 1H); MS (ESI) m/z 352.1 (M⁺+1).

### EXAMPLE 70

### (2-fluorophenyl)(2-{2-[(2R)-2-methyl-1-pyrrolidinyl]ethyl}-1-benzofuran-5-yl)methanone

### EXAMPLE 70A

### (2-fluorophenyl)(4-hydroxy-3-iodophenyl)methanone

(2-Fluorophenyl)(4-hydroxyphenyl)methanone, potassium iodide and iodide chips were processed as described in Example 68A to provide the title compound. MS (DCI) m/z 343 (M+H)⁺, 360 (M+NH₄)⁺.

### EXAMPLE 70B

### (2-fluorophenyl)(2-{2-[(2R)-2-methyl-1-pyrrolidinyl]ethyl}-1-benzofuran-5-yl)methanone

The product from Example 70A and a 0.1M solution of the product from Example 68B were processed as described in Example 68C to provide the title compound. ¹HNMR (300 MHz, CD₃OD) δ1.28 (d, 3H, J=6.1 Hz), 1.59 (m, 1H), 1.93 (m, 2H), 2.10-2.78 (m, 2H), 2.93 (m, 2H), 3.18 (m, 2H), 3.45 (m, 2H), 6.76 (s, 1H), 7.21-7.38 (m, 2H), 7.50-7.67 (m, 3H), 7.79 (d, 1H, J=8.9 Hz), 7.99 (s, 1H); MS (ESI) m/z 352.1 (M+H)⁺.

### EXAMPLE 71

### (3-chlorophenyl)(2-{2-[(2R)-2-methyl-1-pyrrolidinyl]ethyl}-1-benzofuran-5-yl)methanone

### EXAMPLE 71A

### 4-(benzyloxy)benzoyl chloride

4-Benzyloxybenzoic acid (15.0 g, 65.72 mmol) in dichloromethane (150 mL) and dimethylformamide (0.75 mL) was cooled to 0° C. After 30 minutes, the mixture was treated with neat oxalyl chloride (11.5 mL, 131.44 mmol) dropwise over 25 minutes. The resulting mixture was stirred at room temperature for 120 minutes followed by evaporation of solvent under reduced pressure to provide the title compound as a light yellow solid (18.2 g, 112% yield). ¹HNMR (300 MHz, CDCl₃) δ7.03 (d, 2H, J=8.9 Hz), 7.23-7.43 (m, 5H), 8.07 (d, 2H, J=8.9 Hz).

### EXAMPLE 71B

### 4-(benzyloxy)-N-methoxy-N-methylbenzamide

The product from Example 71A (18 g, 72.96 mmol) in DCM at room temperature was treated with N,O-dimethylhydroxylamine hydrochloride (7.83 g, 80.26 mmol) slowly. The mixture was cooled to 0° C., stirred for 30 minutes, and treated with triethylamine (25.47 mL, 182.41 mmol) dropwise. The mixture was allowed to warm to 25° C., stirred for 16 hours, and treated with DCM (150 mL). The mixture was washed with saturated NaHCO₃, brine, and water. The organic phase was dried, filtered, and the filtrate evaporated under reduced pressure to provide the title compound as a pale yellow solid (18.65 g, 95% yield). ¹HNMR (300 MHz, CDCl₃) δ3.36 (s, 3H), 3.56 (s, 3H) 6.98 (d, 2H, J=8.9 Hz), 7.33-7.46 (m, 5H), 7.76 (d, 2H, J=8.9 Hz); MS (ESI) m/z 272 (M+H)⁺.

### EXAMPLE 71C

### 4-hydroxy-N-methoxy-N-methylbenzamide

10% Palladium on charcoal (4.5 g) in methanol (10 mL) was treated with the product from Example 71B (18.60 g, 68.55 mmol) in 150 mL methanol. The mixture was placed under hydrogen atmosphere at 67 psi. The reaction mixture was filtered and the filtrate was evaporated under reduced pressure. The residue was purified by flash chromatography to provide the title compound (10.3 g, 83% yield). ¹HNMR (300 MHz, CD₃OD) δ3.32 (s, 3H), 3.58 (s, 3H), 6.81 (d, 2H, J=8.9 Hz), 7.59 (d, 2H, J=8.9 Hz); MS (DCI) m/z 182 (M+H)⁺, 199 (M+NH₄)⁺.

### EXAMPLE 71D

### 4-hydroxy-3-iodo-N-methoxy-N-methylbenzamide

The product from Example 71C (10.3 g, 56.84 mmol) in concentrated ammonium hydroxide (400 mL) was stirred at 25° C. for 15 minutes and then treated with KI (45.96 g, 276.83 mmol) and I₂ (14.43 g, 56.84 mmol) in water (65 mL). After stirring at room temperature for 16 hours, the solvent was removed under reduced pressure and the residure was redissolved in DCM (500 mL) and washed with water (350 mL×2). The organic phase was dried, filtered, and the filtrate was concentrated under reduced pressure. The residue was purified by flash chromatography on silica gel using 90% CH₂Cl₂, 10% MeOH, to provide the title compound as a white solid (11.6 g, 67% yield). ¹HNMR (300 MHz, CD₃OD) δ3.32 (s, 3H), 3.59 (s, 3H), 6.83 (d, 1H, J=8.9 Hz), 7.58 (d, 1H, J=8.9 Hz), 8.06 (s, 1H); MS (DCI) m/z 308 (M+H)⁺, 325 (M+NH₄)⁺.

### EXAMPLE 71E

### N-methoxy-N-methyl-2-{2-[(2R)-2-methyl-1-pyrrolidinyl]ethyl}-1-benzofuran-5-carboxamide

The product from Example 71D (11.6 g, 37.77 mmol) in acetonitrile (50 mL) was treated sequentially with a 0.12M solution of the product from Example 68B (378 mL, 45.33 mmol), Pd(OAc)₂ (0.254 g, 1.13 mmol), tris(4-methylphenyl)phosphine (0.518 g, 1.699 mmol), and diisopropyl amine (39.7 mL, 283.3 mmol). After stirring at 25° C. for 10 minutes, the mixture was treated with copper iodide (2.158 g, 11.33 mmol) and heated at 50° C. in an inert atmosphere for 18 hours. The reaction mixture was allowed to cool to room temperature, filtered through celite, and the filtrate was concentrated under reduced pressure. The residue was purified on silica gel using 95% DCM, 9.9% MeOH, 0.1% NH₄OH to provide the title compound (1.22 g, 10.2% yield). ¹HNMR (300 MHz, CD₃OD) δ1.18 (d, 3H, J=6.1 Hz), 1.47 (m, 1H), 1.78 (m, 2H), 1.91-2.34 (m, 2H), 2.50 (m, 2H) 3.06 (m, 2H), 3.26 (m, 2H), 3.38 (s, 3H), 3.59 (s, 3H), 6.63 (s, 1H), 7.51 (q, 2H), 7.84 (1H); MS (ESI) m/z 317.2 (M+H)⁺.

### EXAMPLE 71F

### (3-chlorophenyl)(2-{2-[(2R)-2-methyl-1-pyrrolidinyl]ethyl}-1-benzofuran-5-yl)methanone

The product from Example 71E (0.05 g, 0.158 mmol) in 5 mL of anhydrous THF at 0° C. was treated with 3-chlorophenylmagnesium bromide (1.58 mL, 0.79 mmol). The mixture was allowed to slowly warm to 25° C. and stir under nitrogen for 18 hours. The reaction mixture was quenched with saturated ammonium chloride solution and extracted with DCM (50 mL×2). The organic phases were combined, dried, filtered, and the filtrate evaporated under reduced pressure. The residue was purified by preparative HPLC on a Waters Nova-Pak HR C18 column (40 mm×100 mm, 6 μm particle size) using a gradient of 10% to 100% acetonitrile:0.1% aqueous TFA over 12 minute (15 minute run time) at a flow rate of 70 mL/minute to provide the title compound. ¹HNMR (300 MHz, CD₃OD) δ1.50 (d, 3H), 1.72 (m, 1H), 2.10 (m, 2H), 2.35 (m, 1H), 3.30 (m, 4H), 3.55 (m, 1H), 3.80 (m, 2H), 6.90 (s, 1H), 7.50-7.80 (m, 6H), 8.02 (d, 1H); MS (ESI) m/z 368 (M+H)⁺.

### EXAMPLE 72

### (4-chlorophenyl)(2-{2-[(2R)-2-methyl-1-pyrrolidinyl]ethyl}-1-benzofuran-5-yl)methanone

The product from Example 71E and 4-chlorophenylmagnesium bromide were processed as described in Example 71F to provide the title compound. ¹HNMR (300 MHz, CD₃OD) δ1.18 (d, 3H, J=6.1 Hz), 1.46 (m, 1H), 1.78 (m, 2H), 2.01-2.36 (m, 2H), 2.50 (m, 2H), 3.03 (m, 2H), 3.23 (m, 2H), 6.67 (s, 1H), 7.57 (m, 3H), 7.78 (m, 3H), 7.97 (s, 1H); MS (ESI) m/z 368.1 (M⁺+1).

### EXAMPLE 73

### (4-methoxyphenyl)(2-{2-[(2R)-2-methyl-1-pyrrolidinyl]ethyl}-1-benzofuran-5-yl)methanone

The product from Example 71E and 4-methoxyphenylmagnesium bromide were processed as described in Example 71F to provide the title compound. ¹HNMR (CD₃OD) δ1.18 (d, 3H, J=6.1 Hz), 1.46 (m, 1H), 2.01-2.32 (m, 2H), 2.50 (m, 2H), 3.06 (m, 2H), 3.24 (m 2H), 3.88 (s, 3H), 6.67 (s, 1H), 7.05 (d, 2H, J=8.9 Hz), 7.54 (d, 2H, J=8.9 Hz), 7.68 (d, 2H, J=8.9 Hz), 7.80 (d, 2H, J=8.9 Hz), 7.92 (s, 1H); MS (ESI) m/z 364.1 (M⁺+1).

### EXAMPLE 74

### (4-fluoro-3-methylphenyl)(2-{2-[(2R)-2-methyl-1-pyrrolidinyl]ethyl}-1-benzofuran-5-yl)methanone

The product from Example 71E and (4-fluoro-3-methyl)phenylmagnesium bromide were processed as described in Example 71F to provide the title compound. ¹HNMR (300 MHz, CD₃OD) δ1.17 (d, 3H), 1.45 (m, 1H), 1.80 (m, 2H), 2.0 (m, 1H), 2.3 (m, 4H), 2.50 (m, 2H), 3.05 (m, 2H), 3.25 (m, 2H), 6.65 (s, 1H), 7.2 (m, 1H), 7.57 (d, 1H), 7.63 (m, 1H), 7.70 (dd, 2H), 7.95 (d, 1H); MS (ESI) m/z 366 (M+H)⁺.

### EXAMPLE 75

### cyclopropyl(2-{2-[(2R)-2-methyl-1-pyrrolidinyl]ethyl}-1-benzofuran-5-yl)methanone

The product from Example 71E and cyclopropylmagnesium bromide were processed as described in Example 71F to provide the title compound. ¹HNMR (300 MHz, CD₃OD) δ1.10 (m, 5H), 1.28 (d, 3H), 1.78 (m, 1H), 2.10 (m, 2H), 2.38 (m, 1H), 2.9 (m, 1H), 3.2-3.8 (m, 6H), 6.85 (s, 1H), 7.58 (d, 1H), 8.05 (dd, 1H), 8.33 (d, 1H); MS (ESI) m/z 298 (M+H)⁺.

### EXAMPLE 76

### 3-ethyl-1-(2-{2-[(2R)-2-methyl-1-pyrrolidinyl]ethyl}-1-benzofuran-5-yl)-1-pentanone

The product from Example 71E and 2-ethylbutylmagnesium bromide were processed as described in Example 71F to provide the title compound. ¹HNMR (300 MHz, CD₃OD) δ0.9 (m, 6H), 1.23 (m, 1H), 1.20 (m, 6H), 1.75 (m, 1H), 2.1 (m, 2H), 2.35 (m, 1H), 3.05 (m, 1H), 3.2-3.5 (m, 7H), 3.55 (m, 1H), 3.8 (m, 1H), 6.85 (s, 1H), 7.55 (d, 1H) 7.95 (dd, 1H), 8.24 (d, 1H); MS (ESI) m/z 342 (M+H)⁺.

### EXAMPLE 77

### (4-chloro-3-methylphenyl)(2-{2-[(2R)-2-methyl-1-pyrrolidinyl]ethyl}-1-benzofuran-5-yl)methanone

The product from Example 71E and 4-chloro-3-methylphenylmagnesium bromide were processed as described in Example 71F to provide the title compound. ¹HNMR (300 MHz, CD₃OD) δ1.47 (d, 3H), 1.75 (m, 1H), 1.80 (m, 2H), 2.10 (m, 2H), 2.38 (m, 1H), 2.44 (s, 3H), 3.50 (m, 2H), 3.55 (m, 1H), 3.80 (m, 2H), 6.85 (s, 1H), 7.5 (m, 3H), 7.7 (bs, 1H), 7.79 (dd, 1H), 8.01 (d, 1H); MS (ESI) m/z 382 (M+H)⁺.

### EXAMPLE 78

### (2-{2-[(2R)-2-methyl-1-pyrrolidinyl]ethyl}-1-benzofuran-5-yl)[4-(methylthio)phenyl]methanone

The product from Example 71E and 4-(methylthio)phenylmagnesium bromide were processed as described in Example 71F to provide the title compound. ¹HNMR (300 MHz, CD₃OD) δ1.45 (d, 3H), 1.75 (m, 1H), 1.80 (m, 2H), 2.30 (m, 2H), 2.38 (m, 1H), 2.54 (s, 3H), 3.50 (m, 2H), 3.55 (m, 1H), 3.80 (m, 2H), 6.85 (s, 1H), 7.4 (dd, 2H), 7.7 (bs, 1H), 7.6 (dd, 1H), 7.75 (m, 3H), 8.0 (d, 1H); MS (ESI) m/z 380 (M+H)⁺.

### EXAMPLE 79

### [4-(dimethylamino)phenyl](2-{2-[(2R)-2-methyl-1-pyrrolidinyl]ethyl}-1-benzofuran-5-yl)methanone

The product from Example 71E and 4-(dimethylamino)phenylmagnesium bromide were processed as described in Example 71F to provide the title compound. ¹HNMR (300 MHz, CD₃OD) δ1.44 (d, 3H), 1.73 (m, 1H), 1.80 (m, 2H), 2.15 (m, 2H), 2.35 (m, 1H), 3.18 (s, 6H), 3.50 (m, 2H), 3.55 (m, 1H), 3.80 (m, 2H), 6.80 (dd, 2H), 6.85 (s, 1H), 7.56 (dd, 1H), 7.65 (dd, 1H), 7.75 (dd, 2H), 7.95 (d, 1H); MS (ESI) m/z 377 (M+H)⁺.

### EXAMPLE 80

### (4-methylphenyl)(2-{2-[(2R)-2-methyl-1-pyrrolidinyl]ethyl}-1-benzofuran-5-yl)methanone

The product from Example 71E and 4-methylphenylmagnesium bromide were processed as described in Example 71F to provide the title compound. ¹HNMR (300 MHz, CD₃OD) δ1.48 (d, 3H), 1.75 (m, 1H), 2.1 (m, 2H), 2.38 (m, 1H), 2.45 (s, 3H), 3.30 (m, 4H), 3.57 (m, 1H), 3.80 (m, 2H), 6.85 (s, 1H), 7.38 (dd, 2H), 7.60 (dd, 1H), 7.70 (dd, 2H), 7.75 (dd, 1H), 8.0 (d, 1H); MS (ESI) m/z 348 (M+H)⁺.

### EXAMPLE 81

### (3,5-difluorophenyl)(2-{2-[(2R)-2-methyl-1-pyrrolidinyl]ethyl}-1-benzofuran-5-yl)methanone

The product from Example 71E and 3,5-difluorophenylmagnesium bromide were processed as described in Example 71F to provide the title compound. ¹HNMR (300 MHz, CD₃OD) δ1.45 (d, 3H), 1.75 (m, 1H), 2.13 (m, 2H), 2.35 (m, 1H), 3.30 (m, 4H), 3.56 (m, 1H), 3.82 (m, 2H), 6.88 (s, 1H), 7.38 (dd, 2H), 7.30 (m, 3H), 7.63 (dd, 1H), 7.80 (dd, 1H), 8.05 (d, 1H); MS (ESI) m/z 370 (M+H)⁺.

### EXAMPLE 82

### (2-methoxyphenyl)(2-{2-[(2R)-2-methyl-1-pyrrolidinyl]ethyl}-1-benzofuran-5-yl)methanone

The product from Example 71E and 2-methoxyphenylmagnesium bromide were processed as described in Example 71F to provide the title compound. ¹HNMR (300 MHz, CD₃OD) δ1.42 (d, 3H), 1.72 (m, 1H), 2.10 (m, 2H), 2.35 (m, 1H), 3.30 (m, 4H), 3.55 (m, 1H), 3.70 (s 3H), 3.80 (m, 2H), 6.80 (s, 1H), 7.1 (m, 2H), 7.30 (dd, 1H), 7.53 (m, 2H), 7.75 (dd, 1H), 7.95 (d, 1H); MS (ESI) m/z 364 (M+H)⁺.

### EXAMPLE 83

### (3-methoxyphenyl)(2-{2-[(2R)-2-methyl-1-pyrrolidinyl]ethyl}-1-benzofuran-5-yl)methanone

The product from Example 71E and 3-methoxyphenylmagnesium bromide were processed as described in Example 71F to provide the title compound. ¹HNMR (300 MHz, CD₃OD) δ1.42 (d, 3H), 1.72 (m, 1H), 2.10 (m, 2H), 2.35 (m, 1H), 3.30 (m, 4H), 3.55 (m, 1H), 3.80 (m, 2H), 3.83 (s, 3H), 6.83 (s, 1H), 7.2 (m, 1H), 7.30 (m, 2H), 7.45 (m, 1H), 7.60 (m, 2H), 7.80 (dd, 1H), 8.02 (d, 1H); MS (ESI) m/z 364 (M+H)⁺.

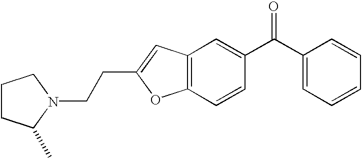

### EXAMPLE 84

### (2-{2-[(2R)-2-methyl-1-pyrrolidinyl]ethyl}-1-benzofuran-5-yl)(phenyl)methanone

The product from Example 71E and phenylmagnesium bromide were processed as described in Example 71F to provide the title compound. ¹HNMR (300 MHz, CD₃OD) δ1.15 (d, 3H), 1.45 (m, 1H), 1.80 (m, 2H), 2.00 (m, 1H), 2.30 (m, 1H), 2.50 (m, 2H), 3.30 (m, 4H), 6.63 (s, 1H), 7.55 (m, 3H), 7.65 (m, 1H), 7.80 (m, 3H), 7.98 (d, 1H); MS (ESI) m/z 334 (M+H)⁺.

Compounds of the present invention may exist as stereoisomers wherein, asymmetric or chiral centers are present. These stereoisomers are “R” or “S” depending on the configuration of substituents around the chiral carbon atom. The terms “R” and “S” used herein are configurations as defined in IUPAC 1974 Recommendations for Section E, Fundamental Stereochemistry, Pure Appl. Chem., 1976, 45: 13-30. The present invention contemplates various stereoisomers and mixtures thereof and are specifically included within the scope of this invention. Stereoisomers include enantiomers and diastereomers, and mixtures of enantiomers or diastereomers. Individual stereoisomers of compounds of the present invention may be prepared synthetically from commercially available starting materials which contain asymmetric or chiral centers or by preparation of racemic mixtures followed by resolution well-known to those of ordinary skill in the art. These methods of resolution are exemplified by (1) attachment of a mixture of enantiomers to a chiral auxiliary, separation of the resulting mixture of diastereomers by recrystallization or chromatography and liberation of the optically pure product from the auxiliary or (2) direct separation of the mixture of optical enantiomers on chiral chromatographic columns.

The term “pharmaceutically acceptable carrier,” as used herein, means a non-toxic, inert solid, semi-solid or liquid filler, diluent, encapsulating material or formulation auxiliary of any type. Some examples of materials which can serve as pharmaceutically acceptable carriers are sugars such as lactose, glucose and sucrose; starches such as corn starch and potato starch; cellulose and its derivatives such as sodium carboxymethyl cellulose, ethyl cellulose and cellulose acetate; powdered tragacanth; malt; gelatin; talc; excipients such as cocoa butter and suppository waxes; oils such as peanut oil, cottonseed oil, safflower oil, sesame oil, olive oil, corn oil and soybean oil; glycols; such a propylene glycol; esters such as ethyl oleate and ethyl laurate; agar; buffering agents such as magnesium hydroxide and aluminum hydroxide; alginic acid; pyrogen-free water; isotonic saline; Ringer's solution; ethyl alcohol, and phosphate buffer solutions, as well as other non-toxic compatible lubricants such as sodium lauryl sulfate and magnesium stearate, as well as coloring agents, releasing agents, coating agents, sweetening, flavoring and perfuming agents, preservatives and antioxidants can also be present in the composition, according to the judgment of one skilled in the art of formulations.

The present invention provides pharmaceutical compositions which comprise compounds of the present invention formulated together with one or more non-toxic pharmaceutically acceptable carriers. The pharmaceutical compositions can be formulated for oral administration in solid or liquid form, for parenteral injection or for rectal administration.

Further included within the scope of the present invention are pharmaceutical compositions comprising one or more of the compounds of formula I-V prepared and formulated in combination with one or more non-toxic pharmaceutically acceptable compositions. The pharmaceutical compositions can be formulated for oral administration in solid or liquid form, for parenteral injection or for rectal administration.

The pharmaceutical compositions of this invention can be administered to humans and other mammals orally, rectally, parenterally , intracisternally, intravaginally, intraperitoneally, topically (as by powders, ointments or drops), bucally or as an oral or nasal spray. The term “parenterally,” as used herein, refers to modes of administration which include intravenous, intramuscular, intraperitoneal, intrasternal, subcutaneous, intraarticular injection and infusion.

Pharmaceutical compositions of this invention for parenteral injection comprise pharmaceutically acceptable sterile aqueous or nonaqueous solutions, dispersions, suspensions or emulsions and sterile powders for reconstitution into sterile injectable solutions or dispersions. Examples of suitable aqueous and nonaqueous carriers, diluents, solvents or vehicles include water, ethanol, polyols (propylene glycol, polyethylene glycol, glycerol, and the like), suitable mixtures thereof, vegetable oils (such as olive oil) and injectable organic esters such as ethyl oleate. Proper fluidity may be maintained, for example, by the use of a coating such as lecithin, by the maintenance of the required particle size in the case of dispersions, and by the use of surfactants.

These compositions may also contain adjuvants such as preservative agents, wetting agents, emulsifying agents, and dispersing agents. Prevention of the action of microorganisms may be ensured by various antibacterial and antifungal agents, for example, parabens, chlorobutanol, phenol, sorbic acid, and the like. It may also be desirable to include isotonic agents, for example, sugars, sodium chloride and the like. Prolonged absorption of the injectable pharmaceutical form may be brought about by the use of agents delaying absorption, for example, aluminum monostearate and gelatin.

In some cases, in order to prolong the effect of a drug, it is often desirable to slow the absorption of the drug from subcutaneous or intramuscular injection. This may be accomplished by the use of a liquid suspension of crystalline or amorphous material with poor water solubility. The rate of absorption of the drug then depends upon its rate of dissolution which, in turn, may depend upon crystal size and crystalline form. Alternatively, delayed absorption of a parenterally administered drug form is accomplished by dissolving or suspending the drug in an oil vehicle.

Suspensions, in addition to the active compounds, may contain suspending agents, as, for example, ethoxylated isostearyl alcohols, polyoxyethylene sorbitol and sorbitan esters, microcrystalline cellulose, aluminum metahydroxide, bentonite, agar-agar, tragacanth, and mixtures thereof.

If desired, and for more effective distribution, the compounds of the present invention can be incorporated into slow-release or targeted-delivery systems such as polymer matrices, liposomes, and microspheres. They may be sterilized, for example, by filtration through a bacteria-retaining filter or by incorporation of sterilizing agents in the form of sterile solid compositions, which may be dissolved in sterile water or some other sterile injectable medium immediately before use.

The active compounds can also be in micro-encapsulated form, if appropriate, with one or more excipients as noted above. The solid dosage forms of tablets, dragees, capsules, pills, and granules can be prepared with coatings and shells such as enteric coatings, release controlling coatings and other coatings well known in the pharmaceutical formulating art. In such solid dosage forms the active compound can be admixed with at least one inert diluent such as sucrose, lactose, or starch. Such dosage forms may also comprise, as is normal practice, additional substances other than inert diluents, e.g., tableting lubricants and other tableting aids such a magnesium stearate and microcrystalline cellulose. In the case of capsules, tablets and pills, the dosage forms may also comprise buffering agents. They may optionally contain opacifying agents and can also be of such composition that they release the active ingredient(s) only, or preferentially, in a certain part of the intestinal tract in a delayed manner. Examples of embedding compositions which can be used include polymeric substances and waxes.

Injectable depot forms are made by forming microencapsulated matrices of the drug in biodegradable polymers such as polylactide-polyglycolide. Depending upon the ratio of drug to polymer and the nature of the particular polymer employed, the rate of drug release can be controlled. Examples of other biodegradable polymers include poly(orthoesters) and poly(anhydrides) Depot injectable formulations are also prepared by entrapping the drug in liposomes or microemulsions which are compatible with body tissues.

The injectable formulations can be sterilized, for example, by filtration through a bacterial-retaining filter or by incorporating sterilizing agents in the form of sterile solid compositions which can be dissolved or dispersed in sterile water or other sterile injectable medium just prior to use.

Injectable preparations, for example, sterile injectable aqueous or oleaginous suspensions may be formulated according to the known art using suitable dispersing or wetting agents and suspending agents. The sterile injectable preparation may also be a sterile injectable solution, suspension or emulsion in a nontoxic, parenterally acceptable diluent or solvent such as a solution in 1,3-butanediol. Among the acceptable vehicles and solvents that may be employed are water, Ringer's solution, U.S.P. and isotonic sodium chloride solution. In addition, sterile, fixed oils are conventionally employed as a solvent or suspending medium. For this purpose any bland fixed oil can be employed including synthetic mono- or diglycerides. In addition, fatty acids such as oleic acid are used in the preparation of injectables.

Solid dosage forms for oral administration include capsules, tablets, pills, powders, and granules. In such solid dosage forms, the active compound is mixed with at least one inert, pharmaceutically acceptable excipient or carrier such as sodium citrate or dicalcium phosphate and/or a) fillers or extenders such as starches, lactose, sucrose, glucose, mannitol, and salicylic acid; b) binders such as carboxymethylcellulose, alginates, gelatin, polyvinylpyrrolidinone, sucrose, and acacia; c) humectants such as glycerol; d) disintegrating agents such as agar-agar, calcium carbonate, potato or tapioca starch, alginic acid, certain silicates, and sodium carbonate; e) solution retarding agents such as paraffin; f) absorption accelerators such as quaternary ammonium compounds; g) wetting agents such as cetyl alcohol and glycerol monostearate; h) absorbents such as kaolin and bentonite clay; and i) lubricants such as talc, calcium stearate, magnesium stearate, solid polyethylene glycols, sodium lauryl sulfate, and mixtures thereof. In the case of capsules, tablets and pills, the dosage form may also comprise buffering agents.

Solid compositions of a similar type may also be employed as fillers in soft and hard-filled gelatin capsules using such excipients as lactose or milk sugar as well as high molecular weight polyethylene glycols and the like.

The solid dosage forms of tablets, dragees, capsules, pills, and granules can be prepared with coatings and shells such as enteric coatings and other coatings well known in the pharmaceutical formulating art. They may optionally contain opacifying agents and can also be of a composition that they release the active ingredient(s) only, or preferentially, in a certain part of the intestinal tract in a delayed manner. Examples of embedding compositions which can be used include polymeric substances and waxes.

Compositions for rectal or vaginal administration are preferably suppositories which can be prepared by mixing the compounds of this invention with suitable non-irritating excipients or carriers such as cocoa butter, polyethylene glycol or a suppository wax which are solid at ambient temperature but liquid at body temperature and therefore melt in the rectum or vaginal cavity and release the active compound.

Liquid dosage forms for oral administration include pharmaceutically acceptable emulsions, microemulsions, solutions, suspensions, syrups and elixirs. In addition to the active compounds, the liquid dosage forms may contain inert diluents commonly used in the art such as, for example, water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethylformamide, oils (in particular, cottonseed, groundnut, corn, germ, olive, castor, and sesame oils), glycerol, tetrahydrofurfuryl alcohol, polyethylene glycols and fatty acid esters of sorbitan, and mixtures thereof.

Besides inert diluents, the oral compositions can also include adjuvants such as wetting agents, emulsifying and suspending agents, sweetening, flavoring, and perfuming agents.

Dosage forms for topical or transdermal administration of a compound of this invention include ointments, pastes, creams, lotions, gels, powders, solutions, sprays, inhalants or patches. The active component is admixed under sterile conditions with a pharmaceutically acceptable carrier and any needed preservatives or buffers as may be required. Ophthalmic formulation, ear drops, eye ointments, powders and solutions are also contemplated as being within the scope of this invention.

The ointments, pastes, creams and gels may contain, in addition to an active compound of this invention, excipients such as animal and vegetable fats, oils, waxes, paraffins, starch, tragacanth, cellulose derivatives, polyethylene glycols, silicones, bentonites, silicic acid, talc and zinc oxide, or mixtures thereof.

Powders and sprays can contain, in addition to the compounds of this invention, excipients such as lactose, talc, silicic acid, aluminum hydroxide, calcium silicates and polyamide powder, or mixtures of these substances. Sprays can additionally contain customary propellants such as chlorofluorohydrocarbons.

Compounds of the present invention may also be administered in the form of liposomes. As is known in the art, liposomes are generally derived from phospholipids or other lipid substances. Liposomes are formed by mono- or multi-lamellar hydrated liquid crystals that are dispersed in an aqueous medium. Any non-toxic, physiologically acceptable and metabolizable lipid capable of forming liposomes may be used. The present compositions in liposome form may contain, in addition to the compounds of the present invention, stabilizers, preservatives, excipients, and the like. The preferred lipids are the natural and synthetic phospholipids and phosphatidylcholines (lecithins) used separately or together.

Methods to form liposomes are known in the art. See, for example, Prescott, Ed., Methods in Cell Biology, Volume XIV, Academic Press, New York, N. Y., (1976), p 33 et seq.

The terms “pharmaceutically acceptable salts, esters and amides,” as used herein, refer to carboxylate salts, amino acid addition salts, zwitterions, esters and amides of compounds of formula I-V which are, within the scope of sound medical judgement, suitable for use in contact with the tissues of humans and lower animals without undue toxicity, irritation, allergic response, and the like, are commensurate with a reasonable benefit/risk ratio, and are effective for their intended use.

The compounds of the present invention can be used in the form of pharmaceutically acceptable salts derived from inorganic or organic acids. By “pharmaceutically acceptable salt” is meant those salts which are, within the scope of sound medical judgement, suitable for use in contact with the tissues of humans and lower animals without undue toxicity, irritation, allergic response and the like and are commensurate with a reasonable benefit/risk ratio. Pharmaceutically acceptable salts are well-known in the art. For example, S. M. Berge et al. describe pharmaceutically acceptable salts in detail in J. Pharmaceutical Sciences, 1977, 66: 1 et seq. The salts can be prepared in situ during the final isolation and purification of the compounds of the invention or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphorsufonate, digluconate, glycerophosphate, hemisulfate, heptanoate, hexanoate, fumarate, hydrochloride, hydrobromide, hydroiodide, 2-hydroxyethansulfonate (isethionate), lactate, maleate, methanesulfonate, nicotinate, 2-naphthalenesulfonate, oxalate, pamoate, pectinate, persulfate, 3-phenylpropionate, picrate, pivalate, propionate, succinate, tartrate, thiocyanate, phosphate, glutamate, bicarbonate, p-toluenesulfonate and undecanoate. Also, the basic nitrogen-containing groups can be quaternized with such agents as lower alkyl halides such as methyl, ethyl, propyl, and butyl chlorides, bromides and iodides; dialkyl sulfates like dimethyl, diethyl, dibutyl and diamyl sulfates; long chain halides such as decyl, lauryl, myristyl and stearyl chlorides, bromides and iodides; arylalkyl halides like benzyl and phenethyl bromides and others. Water or oil-soluble or dispersible products are thereby obtained. Examples of acids which can be employed to form pharmaceutically acceptable acid addition salts include such inorganic acids as hydrochloric acid, hydrobromic acid, sulphuric acid and phosphoric acid and such organic acids as oxalic acid, maleic acid, succinic acid and citric acid.

Basic addition salts can be prepared in situ during the final isolation and purification of compounds of this invention by reacting a carboxylic acid-containing moiety with a suitable base such as the hydroxide, carbonate or bicarbonate of a pharmaceutically acceptable metal cation or with ammonia or an organic primary, secondary or tertiary amine. Pharmaceutically acceptable salts include, but are not limited to, cations based on alkali metals or alkaline earth metals such as lithium, sodium, potassium, calcium, magnesium and aluminum salts and the like and nontoxic quaternary ammonia and amine cations including ammonium, tetramethylammonium, tetraethylammonium, methylamine, dimethylamine, trimethylamine, triethylamine, diethylamine, ethylamine and the like. Other representative organic amines useful for the formation of base addition salts include ethylenediamine, ethanolamine, diethanolamine, piperidine, piperazine and the like. Preferred salts of the compounds of the invention include phosphate, tris and acetate.

The term “pharmaceutically acceptable prodrug” or “prodrug,” as used herein, represents those prodrugs of the compounds of the present invention which are, within the scope of sound medical judgement, suitable for use in contact with the tissues of humans and lower animals without undue toxicity, irritation, allergic response, and the like, commensurate with a reasonable benefit/risk ratio, and effective for their intended use. Prodrugs of the present invention may be rapidly transformed in vivo to a parent compound of formula I-V, for example, by hydrolysis in blood. A thorough discussion is provided in T. Higuchi and V. Stella, Pro-drugs as Novel Delivery Systems, V. 14 of the A.C.S. Symposium Series, and in Edward B. Roche, ed., Bioreversible Carriers in Drug Design, American Pharmaceutical Association and Pergamon Press (1987), hereby incorporated by reference.

Dosage forms for topical administration of a compound of this invention include powders, sprays, ointments and inhalants. The active compound is mixed under sterile conditions with a pharmaceutically acceptable carrier and any needed preservatives, buffers or propellants which can be required. Opthalmic formulations, eye ointments, powders and solutions are also contemplated as being within the scope of this invention.

Actual dosage levels of active ingredients in the pharmaceutical compositions of this invention can be varied so as to obtain an amount of the active compound(s) which is effective to achieve the desired therapeutic response for a particular patient, compositions and mode of administration. The selected dosage level will depend upon the activity of the particular compound, the route of administration, the severity of the condition being treated and the condition and prior medical history of the patient being treated. However, it is within the skill of the art to start doses of the compound at levels lower than required for to achieve the desired therapeutic effect and to gradually increase the dosage until the desired effect is achieved.

The present invention contemplates pharmaceutically active compounds either chemically synthesized or formed by in vivo biotransformation to compounds of formula I-V.

The compounds of the present invention, including but not limited to those specified in the examples, possess an affinity for the histamine-3 receptors. As histamine-3 receptor ligands, the compounds of the present invention may be useful for the treatment and prevention of diseases or conditions such as acute myocardial infarction, Alzheimer's disease, attention-deficit hyperactivity disorder, bipolar disorder, cognitive enhancement, cognitive deficits in psychiatric disorder, drug abuse, deficits of memory and learning, jet lag, Parkinson's disease, epilepsy, schizophrenia, dementia, depression, cutaneous carcinoma, mild cognitive impairment, medullary thyroid carcinoma, melanoma, asthma, narcolepsy, mood and attention alteration, Meniere's disease, gastrointestinal disorders, inflammation, migraine, motion sickness, neurogenic inflammation, obsessive compulsive disorder, Tourette's syndrome, obesity, pain, seizures, septic shock, vertigo, and wakefulness.

The ability of the compounds of the present invention, including but not limited to those specified in the examples, to treat septic shock and cardiovascular disorders, in particular, acute myocardial infarction may be demonstrated by Imamura et al., Circ.Res., (1996) 78, 475-481; Imamura et. al., Circ.Res., (1996) 78, 863-869; R. Levi and N. C. E. Smith, “Hstamine H₃-receptors: A new frontier in myocardial ischemia”, J. Pharm. Exp. Ther., 292: 825-830, (2000); and Hatta, E., K Yasuda and R. Levi, “Activation of histamine H₃ receptors inhibits carrier-mediated norepinephrine release in a human model of protracted myocradial ischemia”, J. Pharm. Exp. Ther., 283: 494-500, (1997).

The ability of the compounds of the invention, including but not limited to those specified in the examples, to treat sleep disorders, in particular, narcolepsy may be demonstrated by Lin et al., Brain Res. (1990) 523, 325-330; Monti et al., Neuropsychopharmacology (1996) 15, 31-35; Sakai, et al., Life Sci. (1991) 48, 2397-2404; Mazurkiewicz-Kwilecki and Nsonwah, Can. J. Physiol. Pharmacol. (1989) 67, 75-78; Panula, P. et al., Neuroscience (1998) 44, 465-481); Wada et al., Trends in Neuroscience (1991) 14, 415; and Monti et al., Eur. J. Pharmacol. (1991) 205, 283.

The ability of the compounds of the invention, including but not limited to those specified in the examples, to treat cognition and memory process disorders may be demonstrated by Mazurkiewicz-Kwilecki and Nsonwah, Can. J. Physiol. Pharmacol. (1989) 67, 75-78; Panula, P. et al., Neuroscience (1997) vol. 82, 993-997; Haas et al., Behav. Brain Res. (1995) 66, 41-44; De Almeida and Izquierdo, Arch. Int. Pharmacodyn. (1986) 283, 193-198; Kamei et al., Psychopharmacology (1990) 102, 312-318; and Kamei and Sakata, Jpn. J. Pharmacol. (1991) 57, 437-482; Schwartz et al., Psychopharmacology; The fourth Generation of Progress. Bloom and Kupfer (eds). Raven Press, New York, (1995) 397; and Wada et al., Trends in Neurosci., (1991) 14, 415.

The ability of the compounds of the invention, including but not limited to those specified in the examples, to treat attention-deficit hyperactivity disorder (ADHD) may be demonstrated by Shaywitz et al., Psychopharmacology (1984) 82, 73-77; Dumery and Blozovski, Exp. Brain Res. (1987) 67, 61-69; Tedford et al., J. Pharmacol. Exp. Ther. (1995) 275, 598-604; and Tedford et al., Soc. Neurosci. Abstr. (1996) 22, 22.

The ability of the compounds of the invention, including but not limited to those specified in the examples, to treat seizures, in particular, epilepsy may be demonstrated by Yokoyama et al., Eur. J. Pharmacol. (1993) 234, 129; Yokoyama and linuma, CNS Drugs (1996) 5, 321; Onodera et al., Prog. Neurobiol. (1994) 42, 685; R. Leurs, R. C. Vollinga and H. Timmerman, “The medicinal chemistry and therapeutic potentials of ligand of the histamine H₃ receptor”, Progress in Drug Research 45: 170-165, (1995); Leurs and Timmerman, Prog. Drug Res. (1992) 39, 127; The Histamine H₃ Receptor, Leurs and Timmerman (eds), Elsevier Science, Amsterdam, The Netherlands (1998); H. Yokoyama and K. Iinuma, “Histamine and Seizures: Implications for the treatment of epilepsy”, CNS Drugs, 5(5); 321-330, (1995); and K. Hurukami, H. Yokoyama, K. Onodera, K. Iinuma and T. Watanabe, AQ-0145, “A newly developed histamine H₃ antagonist, decreased seizure susceptibility of electrically induced convulsions in mice”, Meth. Find. Exp. Clin. Pharmacol., 17(C): 70-73, (1995).

The ability of the compounds of the invention, including but not limited to those specified in the examples, to treat motion sickness, Alzheimer's disease, and Parkinson's disease may be demonstrated by Onodera et al., Prog. Neurobiol. (1994) 42, 685; Leurs and Timmerman, Prog. Drug Res. (1992) 39, 127; and The Histamine H₃ Receptor, Leurs and Timmerman (eds), Elsevier Science, Amsterdam, The Netherlands (1998).

The ability of the compounds of the invention, including but not limited to those specified in the examples, to treat narcolepsy, schizophrenia, depression, and dementia may be demonstrated by R. Leurs, R. C. Vollinga and H. Timmerman, “The medicinal chemistry and therapeutic potentials of ligand of the histamine H₃ receptor”, Progress in Drug Research 45: 170-165, (1995); The Histamine H₃ Receptor, Leurs and Timmerman (eds), Elsevier Science, Amsterdam, The Netherlands (1998); and Perez-Garcia C, et. al., Laboratory of Pharmacology, University of San Pablo CEU, Madrid, Spain, Psychopharmacology (Berl) (1999) February., 142(2): 215-20).

The ability of the compounds of the invention, including but not limited to those specified in the examples, to treat wakefulness, cognitive enhancement, mood and attention alteration, vertigo and motion sickness, and treatment of cognitive deficits in psychiatric disorder may be demonstrated by (Schwartz, Physiol. Review (1991) 71, p. 1-51).

The ability of the compounds of the present invention, including but not limited to those specified in the examples, to treat mild cognitive impairment, deficits of memory, deficits of learning and dementia may be demonstrated by (C. E. Tedford, in “The Histamine H₃ Receptor: a target for new drugs”, the Pharmacochemistry Library, vol. 30 (1998) edited by R. Leurs and H. Timmerman, Elsevier (N.Y.). p. 269 and references also contained therein.)

The ability of the compounds of the invention, including but not limited to those specified in the examples, to treat obesity may be demonstrated by Leurs et al., Trends in Pharm. Sci. (1998) 19, 177-183; Itoh. E., Fujimiay, M., and Inui, A., Thioperamide, A histamine H₃ receptor antagonist, powerfully suppresses peptide YY-induced fodd intake in rats, Biol. Psych. 45(4): 475-481, (1999); Yates S. I., Pawlowski, G. P., Antal, J. M., Ali, S. M., Jiang, J., and Brunden, K. R., Effects of a novel histamine H₃ receptor antagonist, GT-2394, on food intake and weight gain in Sprague-Dawley rats, Abstracts, Society for Neuroscience, 102.10, p. 219, November, (2000); and Bjenning, C., Johannesson, U., Juul, A-G., Lange, K. Z., and Rimvall, K., Peripherally administered ciproxifan elevates hypothalamic histamine levels and potently reduces food intake in the Sprague Dawley rat., Abstracts, International Sendai Histamine Symposium, Sendai, Japan, November, 2000, #P 39.

The ability of the compounds of the invention, including but not limited to those specified in the examples, to treat inflammation and pain may be demonstrated by Phillips et al., Annual Reports in Medicinal Chemistry (1998) 33, 31-40.

The ability of the compounds of the invention, including but not limited to those specified in the examples, to treat migraine may be demonstrated by R. Leurs, R. C. Vollinga and H. Timmerman, “The medicinal chemistry and therapeutic potentials of ligands of the histamine H₃ receptor”, Progress in Drug Research 45: 170-165, (1995); and Matsubara et al., Eur. J. Pharmacol. (1992) 224, 145; and Rouleau et al., J. Pharmacol. Exp. Ther. (1997) 281, 1085.

The ability of the compounds of the invention, including but not limited to those specified in the examples, to treat cancer, in particular, melanoma, cutaneous carcinoma and medullary thyroid carcinoma may be demonstrated by Polish Med. Sci. Mon., (1998) vol. 4, issue 5, 747; Adam Szelag, “Role of histamine H₃-receptors in the proliferation of neoplastic cells in vitro”, Med. Sci. Monit., 4(5): 747-755, (1998); and Fitzsimons, C., H. Duran, F. Labombarda, B. Molinari and E. Rivera, “Histamine receptors signalling in epidermal tumor cell lines with H-ras gene alterations”, Inflammation Res., 47 (Suppl 1): S50-S51, (1998).

The ability of the compounds of the invention, including but not limited to those specified in the examples, to treat vestibular dysfunctions, in particular, Meniere's disease may be demonstrated by R. Leurs, R. C. Vollinga and H. Timmerman, “The medicinal chemistry and therapeutic potentials of ligands of the histamine H³ receptor”, Progress in Drug Research 45: 170-165, (1995).

The ability of the compounds of the present invention, including but not limited to those specified in the examples, to treat asthma may be demonstrated by Delaunois A., Gustin P., Garbarg M., and Ansay M., “Modulation of acetylcholine, capsaicin and substance P effects by histamine H₃ receptors in isolated perfused rabbit lungs”, European Journal of Pharmacology 277(2-3):243-50, (1995); and Dimitriadou, et al., “Functional relationship between mast cells and C-sensitive nerve fibres evidenced by histamine H₃-receptor modulation in rat lung and spleen”, Clinical Science. 87(2):151-63, (1994).

Aqueous liquid compositions of the present invention are particularly useful for the treatment and prevention of asthma, epilepsy, Raynaud's syndrome, male sexual dysfunction, female sexual dysfunction, migraine, pain, eating disorders, urinary incontinence, functional bowel disorders, neurodegeneration and stroke.

When used in the above or other treatments, a therapeutically effective amount of one of the compounds of the present invention can be employed in pure form or, where such forms exist, in pharmaceutically acceptable salt, ester, amide or prodrug form. Alternatively, the compound can be administered as a pharmaceutical composition containing the compound of interest in combination with one or more pharmaceutically acceptable excipients. The phrase “therapeutically effective amount” of the compound of the invention means a sufficient amount of the compound to treat disorders, at a reasonable benefit/risk ratio applicable to any medical treatment. It will be understood, however, that the total daily usage of the compounds and compositions of the present invention will be decided by the attending physician within the scope of sound medical judgement. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time of administration, route of administration, and rate of excretion of the specific compound employed; the duration of the treatment; drugs used in combination or coincidental with the specific compound employed; and like factors well known in the medical arts. For example, it is well within the skill of the art to start doses of the compound at levels lower than required to achieve the desired therapeutic effect and to gradually increase the dosage until the desired effect is achieved.

The total daily dose of the compounds of this invention administered to a human or lower animal may range from about 0.003 to about 30 mg/kg/day. For purposes of oral administration, more preferable doses can be in the range of from about 0.1 to about 15 mg/kg/day. If desired, the effective daily dose can be divided into multiple doses for purposes of administration; consequently, single dose compositions may contain such amounts or submultiples thereof to make up the daily dose.

It is undersrood that the foregoing detailed description and accompanying examples are merely illustrative and are not to be taken as limitations upon the scope of the invention, which is defined solely by the appended claims and their equivalents. Various changes and modifications to the disclosed embodiments will be apparent to those skilled in the art. Such changes and modifications, including without limitation those relating to the chemical structures, substituents, derivatives, intermediates, syntheses, formulations and/or methods of use of the invention, may be made without departing from the spirit and scope thereof. All references cited herein are incorporated by referance. In the case of inconsistencies, the instant disclosure, including definitions, will prevail.

What is claimed is:

1. A compound of formula (I) 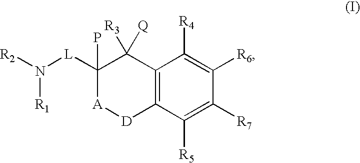
or a pharmaceutically acceptable salt or prodrug thereof, wherein
A is selected from the group consisting of carbonyl and a covalent bond;
D is selected from the group consisting of O and S;
L is selected from the group consisting of —CH₂CH₂— and —CH₂CH₂CH₂—;
P and Q taken together form a covalent bond or are both hydrogen;
R₁ and R₂ are each independently selected from the group consisting of hydrogen, alkyl, aryl, arylalkyl, cycloalkyl, cycloalkylalkyl, heterocycle, heterocyclealkyl, hydroxyalkyl, alkenyl, and alkynyl; or
R₁ and R₂ taken together with the nitrogen atom to which they are attached, together form a heterocycle;
R₃, R₄ and R₅ are each independently selected from the group consisting of hydrogen, alkoxy, alkoxycarbonyl, alkyl, alkylcarbonyl, alkylcarbonyloxy, alkylsulfinyl, alkylsulfonyl, akylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
R₆ and R₇ are each independently selected from the group consisting of hydrogen, alkoxy, alkoxycarbonyl, alkyl, alkylcarbonyl, alkylcarbonyloxy, alkylsulfinyl, alkylsulfonyl, alkylthio, aryl, carboxy, carboxyalkyl, cyano, cyanoalkyl, cycloalkyl, formyl, halogen, haloalkoxy, haloalkyl, heterocycle, hydroxy, hydroxyalkyl, mercapto, nitro, —NR_{A}R_{B}, (NR_{A}R_{B})alkyl, (NR_{A}R_{B})carbonyl, (NR_{A}R_{B})sulfonyl, and —L₂R₂₀;
L₂ is selected from the group consisting of alkylene, alkenylene, O, S, C(═O), N(═OR₂₁) and N(R_{A});
R₂₀ is selected from the group consisting of aryl, heterocycle and cycloalkyl;
R₂₁ is selected from the group consisting of hydrogen and alkyl; and
R_{A} and R_{B} are each independently selected from the group consisting of hydrogen, alkyl, alkylcarbonyl and formyl;
provided that at least one, but not both, of R₆ or R₇ is aryl, heterocycle, cycloalkyl or L₂R₂₀.

2. A compound according to claim 1 wherein
A is a covalent bond;
D is O;
L is —CH₂CH₂—;
P and Q taken together form a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, (2R)-2-methyl-1-pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃, R₄, R₅ and R₇ are hydrogen;
R₆ is L₂R₂₀;
L₂ is C(═O); and
R₂₀ is aryl.

3. A compound according to claim 1 wherein
A is a covalent bond;
D is O;
L is —CH₂CH₂—;
P and Q taken together form a covalent bond;
R₁ and R₂ taken together with the nitrogen atom to which they are attached, together form a heterocycle wherein said heterocycle is (2R)-2-methyl-1-pyrrolidinyl;
R₃, R₄, R₅ and R₇ are hydrogen;
R₆ is L₂R₂₀;
L₂ is C(═O); and
R₂₀ is aryl said aryl is phenyl wherein said phenyl can be substituted with 1, 2 or 3 substitutents selected from the group consisting of alkoxy, alkyl, alkylthio, halogen and —NR_{A}R_{B}.

4. A compound according to claim 3 selected from the group consisting of
(4-fluorophenyl)(2-{2-[(2R)-2-methyl-1-pyrrolidinyl]ethyl}-1-benzofuran-5-yl)methanone;
(3-fluorophenyl)(2-{2-[(2R)-2-methyl-1-pyrrolidinyl]ethyl}-1-benzofuran-5-yl)methanone;
(2-fluorophenyl)(2-{2-[(2R)-2-methyl-1-pyrrolidinyl]ethyl}-1-benzofuran-5-yl)methanone;
(3-chlorophenyl)(2-{2-[(2R)-2-methyl-1-pyrrolidinyl]ethyl}-1-benzofuran-5-yl)methanone;
(4-chlorophenyl)(2-{2-[(2R)-2-methyl-1-pyrrolidinyl]ethyl}-1-benzofuran-5-yl)methanone;
(4-methoxyphenyl)(2-{2-[(2R)-2-methyl-1-pyrrolidinyl]ethyl}-1-benzofuran-5-yl)methanone;
(4-fluoro-3-methylphenyl)(2-{2-[(2R)-2-methyl-1-pyrrolidinyl]ethyl}-1-benzofuran-5-yl)methanone;
(4-chloro-3-methylphenyl)(2-{2-[(2R)-2-methyl-1-pyrrolidinyl]ethyl}-1-benzofuran-5-yl)methanone;
(2-{2-[(2R)-2-methyl-1-pyrrolidinyl]ethyl}-1-benzofuran-5-yl)[4-(methylthio)phenyl]methanone;
[4-(dimethylamino)phenyl](2-{2-[(2R)-2-methyl-1-pyrrolidinyl]ethyl}-1-benzofuran-5-yl)methanone;
(4-methylphenyl)(2-{2-[(2R)-2-methyl-1-pyrrolidinyl]ethyl}-1-benzofuran-5-yl)methanone;
(3,5-difluorophenyl)(2-{2-[(2R)-2-methyl-1-pyrrolidinyl]ethyl}-1-benzofuran-5-yl)methanone;
(2-methoxyphenyl)(2-{2-[(2R)-2-methyl-1-pyrrolidinyl]ethyl}-1-benzofuran-5-yl)methanone;
(3-methoxyphenyl)(2-{2-[(2R)-2-methyl-1-pyrrolidinyl]ethyl}-1-benzofuran-5-yl)methanone; and
(2-{2-[(2R)-2-methyl-1-pyrrolidinyl]ethyl}-1-benzofuran-5-yl)(phenyl)methanone.

5. A compound according to claim 1 wherein
A is a covalent bond;
D is O;
L is —CH₂CH₂—;
P and Q taken together form a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, (2R)-2-methyl-1-pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃, R₄, R₅ and R₇ are hydrogen;
R₆ is L₂R₂₀;
L₂ is C(═O); and
R₂₀ is cycloalkyl.

6. A compound according to claim 1 wherein
A is a covalent bond;
D is O;
L is —CH₂CH₂—;
P and Q taken together form a covalent bond;
R₁ and R₂ taken together with the nitrogen atom to which they are attached, together form a heterocycle wherein said heterocycle is (2R)-2-methyl-1-pyrrolidinyl;
R₃, R₄, R₅ and R₇ are hydrogen; and
R₆ is L₂R₂₀;
L₂ is C(═O); and
R₂₀ is cycloalkyl.

7. A compound according to claim 6 that is cyclopropyl(2-{2-[(2R)-2-methyl-1-pyrrolidinyl]ethyl}-1-benzofuran-5-yl)methanone.

8. A compound according to claim 1 wherein
A is a covalent bond;
D is O;
L is —CH₂CH₂—;
P and Q taken together form a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, (2R)-2-methyl-1-pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃, R₄, R₅ and R₇ are hydrogen; and
R₆ is alkylcarbonyl.

9. A compound according to claim 1 wherein
A is a covalent bond;
D is O;
L is —CH₂CH₂—;
P and Q taken together form a covalent bond;
R₁ and R₂ taken together with the nitrogen atom to which they are attached, together form a heterocycle wherein said heterocycle is (2R)-2-methyl-1-pyrrolidinyl;
R₃, R₄, R₅ and R₇ are hydrogen; and
R₆ is alkylcarbonyl.

10. A compound according to claim 9 that is 3-ethyl-1-(2-{2-[(2R)-2-methyl-1-pyrrolidinyl]ethyl}-1-benzofuran-5-yl)-1-pentanone.

11. A compound according to claim 1 of formula (II) 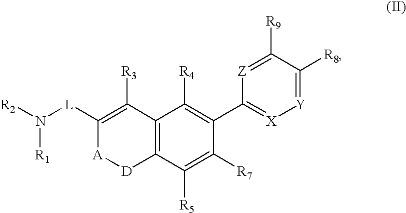
or a pharmaceutical acceptable salt or prodrug thereof, wherein
R₇ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
R₈ is selected from the group consisting of hydrogen, alkylcarbonyl, arylcarbonyl, cyano, cycloalkylcarbonyl, heterocyclecarbonyl and (NR_{A}R_{B})carbonyl;
R₉ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
X is selected from the group consisting of CH, CR_{X} and N;
Y is selected from the group consisting of CH, CR_{Y} and N;
Z is selected from the group consisting of CH, CR_{Z} and N; and
R_{X}, R_{Y} and R_{Z} are each independently selected from the group consisting of alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl.

12. A compound according to claim 11 wherein A is a covalent bond.

13. A compound according to claim 11 wherein
A is a covalent bond;
R₁ and R₂ are each independently selected from the group consisting of hydrogen, alkyl, hydroxyalkyl, alkenyl and alkynyl; and
R₈ is cyano.

14. A compound according to claim 11 wherein
L is —CH₂CH₂—;
A is a covalent bond;
R₁ and R₂ are each independently selected from the group consisting of hydrogen, alkyl, hydroxyalkyl, alkenyl and alkynyl;
R₃, R₄, R₅, R₇ and R₉ are hydrogen;
R₈ is cyano;
X is CH;
Y is CH; and
Z is CH.

15. A compound according to claim 14 selected from the group consisting of:
4-{2-[2-(diethylamino)ethyl]-1-benzofuran-5-yl}benzonitrile;
4-(2-{2-[tert-butyl(methyl)amino]ethyl}-1-benzofuran-5-yl)benzonitrile;
4-(2-{2-[isopropyl(methyl)amino]ethyl}-1-benzofuran-5-yl)benzonitrile;
4-(2-{2-[isobutyl(methyl)amino]ethyl}-1-benzofuran-5-yl)benzonitrile;
4-(2-{2-[ethyl(isopropyl)amino]ethyl}-1-benzofuran-5-yl)benzonitrile; and
4-(2-{2-[ethyl(propyl)amino]ethyl}-1-benzofuran-5-yl)benzonitrile.

16. A compound according to claim 11 wherein
A is a covalent bond;
R₁ and R₂ are each independently selected from the group consisting of hydrogen, alkyl, hydroxyalkyl, alkenyl and alkynyl;
R₈ is cyano;
X is nitrogen;
Y is CH; and
Z is CH.

17. A compound according to claim 11 wherein
A is a covalent bond;
R₁ and R₂ are each independently selected from the group consisting of hydrogen, alkyl, hydroxyalkyl, alkenyl and alkynyl; and
R₈ is heterocyclecarbonyl.

18. A compound according to claim 11 wherein
A is a covalent bond;
R₁ and R₂ are each independently selected from the group consisting of hydrogen, alkyl, hydroxyalkyl, alkenyl and alkynyl; and
R₈ is heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

19. A compound according to claim 11 wherein
A is a covalent bond;
R₁ and R₂ are each independently selected from the group consisting of hydrogen, alkyl, hydroxyalkyl, alkenyl and alkynyl; and
R₈ is heterocyclecarbonyl wherein the heterocycle part of said heterocyclecarbonyl is morpholinyl.

20. A compound according to claim 11 wherein
L is —CH₂CH₂—;
A is a covalent bond;
R₁ and R₂ are each independently selected from the group consisting of hydrogen, alkyl, hydroxyalkyl, alkenyl and alkynyl;
R₃, R₄, R₅, R₇ and R₉ are hydrogen;
R₈ is heterocyclecarbonyl wherein the heterocycle part of said heterocyclecarbonyl is morpholinyl;
X is CH;
Y is CH; and
Z is CH.

21. A compound according to claim 20 selected from the group consisting of:
N,N-diethyl-N-(2-{5-[4-(4-morpholinylcarbonyl)phenyl]-1-benzofuran-2-yl}ethyl)amine;
N-(tert-butyl)-N-methyl-N-(2-{5-[4-(4-morpholinylcarbonyl)phenyl]-1-benzofuran-2-yl}ethyl)amine;
N-isopropyl-N-methyl-N-(2-{5-[4-(4-morpholinylcarbonyl)phenyl]-1-benzofuran-2-yl}ethyl)amine;
N-isobutyl-N-methyl-N-(2-{5-[4-(4-morpholinylcarbonyl)phenyl]-1-benzofuran-2-yl}ethyl)amine;
N-ethyl-N-isopropyl-N-(2-{5-[4-(4-morpholinylcarbonyl)phenyl]-1-benzofuran-2-yl}ethyl)amine;
N,N-dimethyl-N-(2-{5-[4-(4-morpholinylcarbonyl)phenyl]-1-benzofuran-2-yl}ethyl)amine; and
N-ethyl-N-(2-{5-[4-(4-morpholinylcarbonyl)phenyl]-1-benzofuran-2-yl}ethyl)-N-propylamine.

22. A compound according to claim 11 wherein
A is a covalent bond;
R₁ and R₂ are each independently selected from the group consisting of hydrogen, alkyl, hydroxyalkyl, alkenyl and alkynyl;
R₈ is heterocyclecarbonyl;
X is nitrogen;
Y is CH; and
Z is CH.

23. A compound according to claim 11 wherein
A is a covalent bond;
R₁ and R₂ are each independently selected from the group consisting of hydrogen, alkyl, hydroxyalkyl, alkenyl and alkynyl;
R₈ is heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
X is nitrogen;
Y is CH; and
Z is CH.

24. A compound according to claim 11 wherein
A is a covalent bond;
R₁ and R₂ are each independently selected from the group consisting of hydrogen, alkyl, hydroxyalkyl, alkenyl and alkynyl;
R₈ is heterocyclecarbonyl wherein the heterocycle part of said heterocyclecarbonyl is morpholinyl; and
X is nitrogen;
Y is CH; and
Z is CH.

25. A compound according to claim 11 wherein
L is —CH₂CH₂—;
A is a covalent bond;
R₁ and R₂ are each independently selected from the group consisting of hydrogen, alkyl, hydroxyalkyl, alkenyl and alkynyl;
R₃, R₄, R₅, R₇ and R₉ are hydrogen;
R₈ is heterocyclecarbonyl wherein the heterocycle part of said heterocyclecarbonyl is morpholinyl;
X is nitrogen;
Y is CH; and
Z is CH.

26. A compound according to claim 25 selected from the group consisting of:
4-[(6-{2-[2-(N,N-diethyl)ethyl]-1-benzofuran-5-yl}-3-pyridinyl)carbonyl]morpholine;
N-(tert-butyl)-N-methyl-N-(2-{5-[5-(4-morpholinylcarbonyl)-2-pyridinyl]-1-benzofuran-2-yl}ethyl)amine;
N-isobutyl-N-methyl-N-(2-{5-[5-(4-morpholinylcarbonyl)-2-pyridinyl]-1-benzofuran-2-yl}ethyl)amine;
N-isopropyl-N-methyl-N-(2-{5-[5-(4-morpholinylcarbonyl)-2-pyridinyl]-1-benzofuran-2-yl}ethyl)amine;
N-ethyl-N-isopropyl-N-(2-{5-[5-(4-morpholinylcarbonyl)-2-pyridinyl]-1-benzofuran-2-yl}ethyl)amine;
N,N-dimethyl-N-(2-{5-[5-(4-morpholinylcarbonyl)-2-pyridinyl]-1-benzofuran-2-yl}ethyl)amine;
N-ethyl-N-propyl-N-(2-{5-[5-(4-morpholinylcarbonyl)-2-pyridinyl]-1-benzofuran-2-yl}ethyl)amine;
N-allyl-N-(2-{5-[5-(4-morpholinylcarbonyl)-2-pyridinyl]-1-benzofuran-2-yl}ethyl)amine;
3-[(2-{5-[5-(4-morpholinylcarbonyl)-2-pyridinyl]-1-benzofuran-2-yl}ethyl)amino]-1-propanol; and
N-(2-{5-[5-(4-morpholinylcarbonyl)-2-pyridinyl]-1-benzofuran-2-yl}ethyl)-N-propylamine.

27. A compound according to claim 11 wherein
A is a covalent bond; and
R₁ and R₂ taken together with the nitrogen atom to which they are attached, together form a heterocycle.

28. A compound according to claim 11 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is cyano.

29. A compound according to claim 11 wherein
L is —CH₂CH₂—;
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃, R₄, R₅, R₇ and R₉ are hydrogen;
R₈ is cyano;
X is CH;
Y is CH; and
Z is CH.

30. A compound according to claim 29 selected from the group consisting of:
4-{2-[2-(1-pyrrolidinyl)ethyl]-1-benzofuran-5-yl}benzonitrile;
4-{2-[2-(2-methyl-1-pyrrolidinyl)ethyl]-1-benzofuran-5-yl}benzonitrile;
4-{2-[2-(1-piperidinyl)ethyl]-1-benzofuran-5-yl}benzonitrile;
4-{2-[2-(2-methyl-1-piperidinyl)ethyl]-1-benzofuran-5-yl}benzonitrile;
4-(2-{2-[(3R)-3-hydroxypyrrolidinyl]ethyl}-1-benzofuran-5-yl)benzonitrile;
4-{2-[2-(2H-imidazol-1-yl)ethyl]-1-benzofuran-5-yl}benzonitrile;
4-(2-{2-[(3S)-3-(dimethylamino)pyrrolidinyl]ethyl}-1-benzofuran-5-yl)benzonitrile;
4-(2-{2-[(2S)-2-(hydroxymethyl)pyrrolidinyl]ethyl}-1-benzofuran-5-yl)benzonitrile;
4-(2-{2-[(2R,6S)-2,6-dimethylpiperidinyl]ethyl}-1-benzofuran-5-yl)benzonitrile;
4-(2-{2-[(2R,5R)-2,5-dimethylpyrrolidinyl]ethyl}-1-benzofuran-5-yl)benzonitrile;
4-{2-[2-(1-azepanyl)ethyl]-1-benzofuran-5-yl}benzonitrile;
4-{2-[2-(4-methyl-1-piperidinyl)ethyl]-1-benzofuran-5-yl}benzonitrile;
4-(2-{2-[2-pyrrolidine methyl carboxylate]ethyl}-1-benzofuran-5-yl)benzonitrile;
4-{2-[2-(3,6-dihydro-1(2H)-pyridinyl)ethyl]-1-benzofuran-5-yl}benzonitrile;
4-(2-{2-[(2R)-2-(hydroxymethyl)pyrrolidinyl]ethyl}-1-benzofuran-5-yl)benzonitrile;
4-(2-{2-[(3R)-(dimethylamino)pyrrolidinyl]ethyl}-1-benzofuran-5-yl)benzonitrile;
4-(2-{2-[1-(2S)-2-methylpyrrolidinyl]ethyl}-1-benzofuran-5-yl)benzonitrile; and
4-(2-{2-[1-(2-methylpyrrolidinyl]ethyl}-1-benzofuran-5-yl)benzonitrile.

31. A compound according to claim 29 that is 4-(2-{2-[1-(2R)-2-methylpyrrolidinyl]ethyl}-1-benzofuran-5-yl)benzonitrile.

32. A compound according to claim 11 wherein
A is a covalent bond;
R₁ and R₂ taken together with the nitrogen atom to which they are attached, together form a heterocycle;
R₈ is cyano;
X is nitrogen;
Y is CH; and
Z is CH.

33. A compound according to claim 11 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₈ is cyano;
X is nitrogen;
Y is CH; and
Z is CH.

34. A compound according to claim 11 wherein
A is a covalent bond;
R₁ and R₂ taken together with the nitrogen atom to which they are attached, together form a heterocycle; and
R₈ is heterocyclecarbonyl.

35. A compound according to claim 11 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

36. A compound according to claim 11 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is heterocyclecarbonyl wherein the heterocycle part of said heterocyclecarbonyl is morpholinyl.

37. A compound according to claim 11 wherein
L is —CH₂CH₂—;
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃, R₄, R₅, R₇ and R₉ are hydrogen;
R₈ is heterocyclecarbonyl wherein the heterocycle part of said heterocyclecarbonyl is morpholinyl;
X is CH;
Y is CH; and
Z is CH.

38. A compound according to claim 37 selected from the group consisting of:
4-(4-{2-[2-(2-methyl-1-pyrrolidinyl)ethyl]-1-benzofuran-5-yl}benzoyl)morpholine;
4-(4-{2-[2-(1-piperidinyl)ethyl]-1-benzofuran-5-yl}benzoyl)morpholine;
4-(4-{2-[2-(2-methyl-1-piperidinyl)ethyl]-1-benzofuran-5-yl}benzoyl)morpholine;
(3R)-1-(2-{5-[4-(4-morpholinylcarbonyl)phenyl]-1-benzofuran-2-yl}ethyl)-3-pyrrolidinol;
4-[4-(2-{2-[(2R,5R)-2,5-dimethylpyrrolidinyl]ethyl}-1-benzofuran-5-yl)benzoyl]morpholine;
4-[4-(2-{2-[(2R,6S)-2,6-dimethylpiperidinyl]ethyl}-1-benzofuran-5-yl)benzoyl]morpholine;
4-(4-{2-[2-(azepinyl)ethyl]-1-benzofuran-5-yl}benzoyl)morpholine;
4-(4-{2-[2-(4-methyl-1-piperidinyl)ethyl]-1-benzofuran-5-yl}benzoyl)morpholine;
4-(4-{2-[2-(4-morpholine)ethyl]-1-benzofuran-5-yl}benzoyl)morpholine;
4-(4-{2-[2-(3,6-dihydro-1(2H)-pyridinyl)ethyl]-1-benzofuran-5-yl}benzoyl)morpholine; and
4-(4-{2-[2-(2S)-pyrrolidinylmethanol)ethyl]-1-benzofuran-5-yl}benzoyl)morpholine.

39. A compound according to claim 11 wherein
A is a covalent bond;
R₁ and R₂ taken together with the nitrogen atom to which they are attached, together form a heterocycle;
R₈ is heterocyclecarbonyl;
X is nitrogen;
Y is CH; and
Z is CH.

40. A compound according to claim 11 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₈ is heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
X is nitrogen;
Y is CH; and
Z is CH.

41. A compound according to claim 11 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₈ is heterocyclecarbonyl wherein the heterocycle part of said heterocyclecarbonyl is morpholinyl;
X is nitrogen;
Y is CH; and
Z is CH.

42. A compound according to claim 11 wherein
L is —CH₂CH₂—;
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃, R₄, R₅, R₇ and R₉ are hydrogen;
R₈ is heterocyclecarbonyl wherein the heterocycle part of said heterocyclecarbonyl is morpholinyl;
X is nitrogen;
Y is CH; and
Z is CH.

43. A compound according to claim 42 selected from the group consisting of:
4-[(6-{2-[2-(1-pyrrolidinyl)ethyl]-1-benzofuran-5-yl}-3-pyridinyl)carbonyl]morpholine;
4-{[6-(2-{2-[(2R)-methylpyrrolidinyl]ethyl}-1-benzofuran-5-yl)-3-pyridinyl]carbonyl}morpholine;
4-[(6-{2-[2-(1-piperidinyl)ethyl]-1-benzofuran-5-yl}-3-pyridinyl)carbonyl]morpholine;
(3R)-1-(2-{5-[5-(4-morpholinylcarbonyl)-2-pyridinyl]-1-benzofuran-2-yl}ethyl)-3-pyrrolidinol;
4-{[6-(2-{2-[(2R,5R)-2,5-dimethylpyrrolidinyl]ethyl}-1-benzofuran-5-yl)-3-pyridinyl]carbonyl}morpholine;
4-{[6-(2-{2-[(2R,6S)-2,6-dimethylpiperidinyl]ethyl}-1-benzofuran-5-yl)-3-pyridinyl]carbonyl}morpholine;
4-{[6-(2-{2-[1-azepanyl]ethyl}-1-benzofuran-5-yl)-3-pyridinyl]carbonyl}morpholine;
4-[(6-{2-[2-(4-methyl-1-piperidinyl)ethyl]-1-benzofuran-5-yl}-3-pyridinyl)carbonyl]morpholine;
4-[(6-{2-[2-(4-morpholinyl)ethyl]-1-benzofuran-5-yl}-3-pyridinyl)carbonyl]morpholine;
8-(2-{5-[5-(4-morpholinylcarbonyl)-2-pyridinyl]-1-benzofuran-2-yl}ethyl)-1,4-dioxa-8-azaspiro[4.5]decane;
5-(2-{5-[5-(4-morpholinylcarbonyl)-2-pyridinyl]-1-benzofuran-2-yl}ethyl)-2-oxa-5-azabicyclo[2.2.1]heptane; and
(2S)-1-(2-{5-[5-(4-morpholinylcarbonyl)-2-pyridinyl]-1-benzofuran-2-yl }ethyl)-2-pyrrolidinol.

44. A compound according to claim 11 wherein
A is carbonyl;
R₁ and R₂ are each independently selected from the group consisting of hydrogen, alkyl, hydroxyalkyl, alkenyl and alkynyl; and
R₈ is selected from the group consisting of cyano and heterocyclecarbonyl.

45. A compound according to claim 11 wherein
A is carbonyl;
R₁ and R₂ are each independently selected from the group consisting of hydrogen, alkyl, hydroxyalkyl, alkenyl and alkynyl; and
R₈ is selected from the group consisting of cyano and heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

46. A compound according to claim 11 wherein
A is carbonyl;
R₁ and R₂ taken together with the nitrogen atom to which they are attached, together form a heterocycle; and
R₈ is selected from the group consisting of cyano and heterocyclecarbonyl.

47. A compound according to claim 11 wherein
A is carbonyl;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is selected from the group consisting of cyano, or heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

48. A compound according to claim 1 of formula (III) 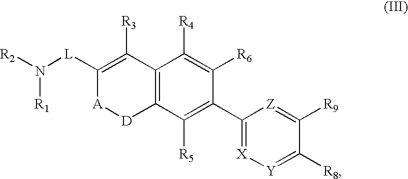
or a pharmaceautical acceptable salt or prodrug thereof, wherein
R₆ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
R₈ is selected from the group consisting of hydrogen, alkylcarbonyl, arylcarbonyl, cyano, cycloalkylcarbonyl, heterocyclecarbonyl and (NR_{A}R_{B})carbonyl;
R₉ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
X is selected from the group consisting of CH, CR_{X} and N;
Y is selected from the group consisting of CH, CR_{Y} and N;
Z is selected from the group consisting of CH, CR_{Z} and N; and
R_{X}, R_{Y} and R_{Z} are each independently selected from the group consisting of alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl.

49. A compound according to claim 48 wherein A is a covalent bond.

50. A compound according to claim 48 wherein
A is a covalent bond;
R₁ and R₂ are each independently selected from the group consisting of hydrogen, alkyl, hydroxyalkyl, alkenyl and alkynyl; and
R₈ is selected from the group consisting of cyano and heterocyclecarbonyl.

51. A compound according to claim 48 wherein
A is a covalent bond;
R₁ and R₂ are each independently selected from the group consisting of hydrogen, alkyl, hydroxyalkyl, alkenyl and alkynyl; and
R₈ is selected from the group consisting of cyano and heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

52. A compound according to claim 48 wherein
A is a covalent bond;
R₁ and R₂ taken together with the nitrogen atom to which they are attached, together form a heterocycle; and
R₈ is selected from the group consisting of cyano and heterocyclecarbonyl.

53. A compound according to claim 48 wherein
A is a covalent bond;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is selected from the group consisting of cyano and heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

54. A compound according to claim 48 wherein A is carbonyl.

55. A compound according to claim 48 wherein
A is carbonyl;
R₁ and R₂ are each independently selected from the group consisting of hydrogen, alkyl, hydroxyalkyl, alkenyl and alkynyl; and
R₈ is cyano.

56. A compound according to claim 48 wherein
L is —CH₂CH₂—;
A is carbonyl;
R₁ and R₂ are each independently selected from the group consisting of hydrogen, alkyl, hydroxyalkyl, alkenyl and alkynyl;
R₃ is methyl;
R₄, R₅, R₆ and R₉ are hydrogen;
R₈ is cyano;
X is CH;
Y is CH; and
Z is CH.

57. A compound according to claim 56 that is 4-{3-[2-(diethylamino)ethyl]-4-methyl-2-oxo-2H-chromen-7-yl}benzonitrile.

58. A compound according to claim 48 wherein
A is carbonyl;
R₁ and R₂ are each independently selected from the group consisting of hydrogen, alkyl, hydroxyalkyl, alkenyl and alkynyl; and
R₈ is heterocyclecarbonyl.

59. A compound according to claim 48 wherein
A is carbonyl;
R₁ and R₂ are each independently selected from the group consisting of hydrogen, alkyl, hydroxyalkyl, alkenyl and alkynyl; and
R₈ is heterocyclecarbonyl wherein the heterocycle part of said heterocyclecarbonyl is selected from the group consisting of azetidinyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

60. A compound according to claim 48 wherein
A is carbonyl;
R₁ and R₂ taken together with the nitrogen atom to which they are attached, together form a heterocycle; and
R₈ is cyano.

61. A compound according to claim 48 wherein
A is carbonyl;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is cyano.

62. A compound according to claim 48 wherein
L is —CH₂CH₂—;
A is carbonyl;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl;
R₃ is methyl;
R₄, R₅, R₆ and R₉ are hydrogen;
R₈ is cyano;
X is CH;
Y is CH; and
Z is CH.

63. A compound according to claim 62 selected from the group consisting of
4-{4-methyl-2-oxo-3-[2-(1-pyrrolidinyl)ethyl]-2H-chromen-7-yl}benzonitrile; and
4-{4-methyl-2-oxo-3-[2-(1-piperidinyl)ethyl]-2H-chromen-7-yl}benzonitrile.

64. A compound according to claim 48 wherein
A is carbonyl;
R₁ and R₂ taken together with the nitrogen atom to which they are attached, together form a heterocycle; and
R₈ is heterocyclecarbonyl.

65. A compound according to claim 48 wherein
A is carbonyl;
R₁ and R₂ taken together with the nitrogen atom to which they are attached, together form a heterocycle selected from the group consisting of azepanyl, azetidinyl, imadazolyl, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl; and
R₈ is heterocyclecarbonyl wherein the heterocycle part of said heterocyclecarbonyl is selected from the group consisting of azetidine, morpholinyl, piperazinyl, piperidinyl, pyridinyl, pyrrolidinyl, 2,5-dihydro-1H-pyrrolyl, pyrrolyl, 3,6-dihydro-1(2H)-pyridinyl, thiomorpholinyl and 1,1-dioxidothiomorpholinyl.

66. A compound according to claim 1 of formula (IV) 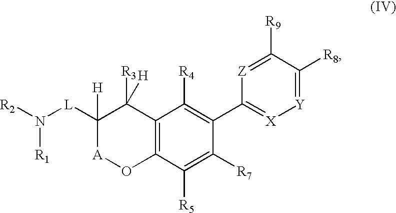
or a pharmaceutical acceptable salt or prodrug thereof, wherein
R₇ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
R₈ is selected from the group consisting of hydrogen, alkylcarbonyl, arylcarbonyl, cyano, cycloalkylcarbonyl, heterocyclecarbonyl and (NR_{A}R_{B})carbonyl;
R₉ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
X is selected from the group consisting of CH, CR_{X} and N;
Y is selected from the group consisting of CH, CR_{Y} and N;
Z is selected from the group consisting of CH, CR_{Z} and N; and
R_{X}, R_{Y} and R_{Z} are each independently selected from the group consisting of alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl.

67. A compound according to claim 66 wherein A is a covalent bond.

68. A compound according to claim 66 wherein
A is a covalent bond;
R₁ and R₂ are each independently selected from the group consisting of hydrogen, alkyl, hydroxyalkyl, alkenyl and alkynyl; and
R₈ is cyano.

69. A compound according to claim 66 wherein
L is —CH₂CH₂—;
A is a covalent bond;
R₁ and R₂ are each independently selected from the group consisting of hydrogen, alkyl, hydroxyalkyl, alkenyl and alkynyl;
R₃, R₄, R₅, R₇ and R₉ are hydrogen;
R₈ is cyano;
X is CH;
Y is CH; and
Z is CH.

70. A compound according to claim 69 that is 4-(2-{2-[(2R)-2-methylpyrrolidinyl]ethyl}-2,3-dihydro-1-benzofuran-5-yl)benzonitrile.

71. A compound according to claim 1 of formula (V) 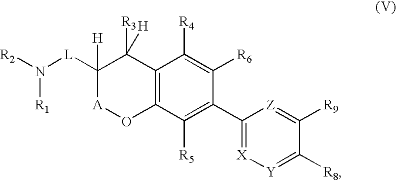
or a pharmaceutical acceptable salt or prodrug thereof, wherein
R₇ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
R₈ is selected from the group consisting of hydrogen, alkylcarbonyl, arylcarbonyl, cyano, cycloalkylcarbonyl, heterocyclecarbonyl and (NR_{A}R_{B})carbonyl;
R₉ is selected from the group consisting of hydrogen, alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl;
X is selected from the group consisting of CH, CR_{X} and N;
Y is selected from the group consisting of CH, CR_{Y} and N;
Z is selected from the group consisting of CH, CR_{Z} and N; and
R_{X}, R_{Y} and R_{Z} are each independently selected from the group consisting of alkoxy, alkoxycarbonyl, alkyl, alkylcarbonyl, alkylcarbonyloxy, alkylsulfinyl, alkylsulfonyl, alkylthio, carboxy, carboxyalkyl, cyano, cyanoalkyl, formyl, halogen, haloalkoxy, haloalkyl, hydroxy, hydroxyalkyl, mercapto, nitro, —NR_{A}R_{B}, (NR_{A}R_{B})alkyl, (NR_{A}R_{B})carbonyl and (NR_{A}R_{B})sulfonyl.

72. A pharmaceutical composition comprising a therapeutically effective amount of a compound of claim 1 in combination with a pharmaceutically acceptable carrier.

73. A method of selectively modulating the effects of histamine-3 receptors in a mammal comprising administering an effective amount of a compound of claim 1.

74. A method of treating a disorder wherein the disorder is ameliorated by modulating the histamine-3 receptors in a mammal comprising administering an effective amount of a compound of claim 1.

75. The method according to claim 74 wherein the disorder is selected from the group consisting of acute myocardial infarction, asthma, bipolar disorder, cognitive enhancement, cognitive deficits in psychiatric disorders, cutaneous carcinoma, drug abuse, depression, gastrointestinal disorders, inflammation, jet lag, medullary thyroid carcinoma, melanoma, Meniere's disease, migraine, mood and attention alteration, motion sickness, neurogenic inflammation, obsessive compulsive disorder, pain, Parkinson's disease, schizophrenia, seizures, septic shock, Tourette's syndrome, vertigo, and wakefulness.

76. The method according to claim 74 wherein the disorder is Alzheimer's disease.

77. The method according to claim 74 wherein the disorder is attention-deficit hyperactivity disorder.

78. The method according to claim 74 wherein the disorder is epilepsy.

79. The method according to claim 74 wherein the disorder is narcolepsy.

80. The method according to claim 74 wherein the disorder is obesity.

81. The method of claim 74 wherein the disorder is selected from the group consisting of mild cognitive impairment, deficits of memory, deficits of learning and dementia.
